(19) United States
(12) Patent Application Publication
(10) Pub. No.: US 2009/0318500 A1
(43) Pub. Date: Dec. 24, 2009

(54) 4-(2, 6-Dichloro-benzoylamino)-1H-pyrazole-3-carboxylic acid (1-methanesulphonyl-piperidin-4-yl)-amide for the Treatment of Cancer

(75) Inventors: Gary Trewartha, Stevenage (GB); Eva Figueroa Navarro, Cambridge (GB); David Charles Rees, Cambridge (GB); Mladen Vinkovic, Cambridge (GB); Andrew James Woodhead, Cambridge (GB); Paul Graham Wyatt, Perth (GB)

Correspondence Address: HESLIN ROTHENBERG FARLEY & MESITI PC, 5 COLUMBIA CIRCLE, ALBANY, NY, 12203, US

(73) Assignee: Astex Therapeutics limited, Cambridge (GB)

(21) Appl. No.: 12/299,723

(22) PCT Filed: May 4, 2007
(86) PCT No.: PCT/GB2007/001655

§ 371 Date: Mar. 6, 2009

Related U.S. Application Data
(60) Provisional application No. 60/746,541, filed on May 5, 2006.
(60) Provisional application No. 60/830,967, filed on Jul. 14, 2006.

(51) Int. Cl. A61K 31/454; C07D 401/12; A61P 35/00

(52) U.S. Cl. 514/326; 546/211

(57) ABSTRACT

The invention provides the compound of formula (I) 4-(2,6-dichloro-benzoylamino)-1H-pyrazole-3-carboxylic acid (1-methanesulphonyl-piperidin-4-yl)-amide in a substantially crystalline form, therapeutic uses thereof and pharmaceutical compositions containing the crystalline compound. The invention also provides novel pharmaceutical formulations containing 4-(2,6-dichloro-benzoylamino)-1H-pyrazole-3-carboxylic acid (1-methanesulphonyl-piperidin-4-yl)-amide and novel processes for preparing the compound.

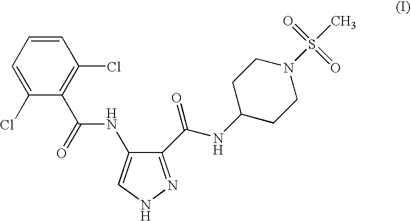

This invention relates to a process for preparing the compound 4-(2,6-dichloro-benzoylamino)-1H-pyrazole-3-carboxylic acid (1-methanesulphonyl-piperidin-4-yl)-amide, pharmaceutical compositions containing the compound and a crystalline form of the compound, as well as the therapeutic uses of the compound.

### BACKGROUND OF THE INVENTION

Protein kinases constitute a large family of structurally related enzymes that are responsible for the control of a wide variety of signal transduction processes within the cell (Hardie, G. and Hanks, S. (1995) The Protein Kinase Facts Book. I and II, Academic Press, San Diego, Calif.). The kinases may be categorized into families by the substrates they phosphorylate (e.g., protein-tyrosine, protein-serine/threonine, lipids, etc.). Sequence motifs have been identified that generally correspond to each of these kinase families (e.g., Hanks, S. K., Hunter, T., FASEB J., 9:576-596 (1995); Knighton, et al., Science, 253:407-414 (1991); Hiles, et al., Cell, 70:419-429 (1992); Kunz, et al., Cell, 73:585-596 (1993); Garcia-Bustos, et al., EMBO J., 13:2352-2361 (1994)).

Protein kinases may be characterized by their regulation mechanisms. These mechanisms include, for example, autophosphorylation, transphosphorylation by other kinases, protein-protein interactions, protein-lipid interactions, and protein-polynucleotide interactions. An individual protein kinase may be regulated by more than one mechanism.

Kinases regulate many different cell processes including, but not limited to, proliferation, differentiation, apoptosis, motility, transcription, translation and other signalling processes, by adding phosphate groups to target proteins. These phosphorylation events act as molecular on/off switches that can modulate or regulate the target protein biological function. Phosphorylation of target proteins occurs in response to a variety of extracellular signals (hormones, neurotransmitters, growth and differentiation factors, etc.), cell cycle events, environmental or nutritional stresses, etc. The appropriate protein kinase functions in signalling pathways to activate or inactivate (either directly or indirectly), for example, a metabolic enzyme, regulatory protein, receptor, cytoskeletal protein, ion channel or pump, or transcription factor. Uncontrolled signalling due to defective control of protein phosphorylation has been implicated in a number of diseases, including, for example, inflammation, cancer, allergy/asthma, diseases and conditions of the immune system, diseases and conditions of the central nervous system, and angiogenesis.

Cyclin Dependent Kinases

The process of eukaryotic cell division may be broadly divided into a series of sequential phases termed G1, S, G2 and M. Correct progression through the various phases of the cell cycle has been shown to be critically dependent upon the spatial and temporal regulation of a family of proteins known as cyclin dependent kinases (cdks) and a diverse set of their cognate protein partners termed cyclins. Cdks are cdc2 (also known as cdk1) homologous serine-threonine kinase proteins that are able to utilise ATP as a substrate in the phosphorylation of diverse polypeptides in a sequence dependent context. Cyclins are a family of proteins characterised by a homology region, containing approximately 100 amino acids, termed the “cyclin box” which is used in binding to, and defining selectivity for, specific cdk partner proteins.

Modulation of the expression levels, degradation rates, and activation levels of various cdks and cyclins throughout the cell cycle leads to the cyclical formation of a series of cdk/cyclin complexes, in which the cdks are enzymatically active. The formation of these complexes controls passage through discrete cell cycle checkpoints and thereby enables the process of cell division to continue. Failure to satisfy the pre-requisite biochemical criteria at a given cell cycle checkpoint, i.e. failure to form a required cdk/cyclin complex, can lead to cell cycle arrest and/or cellular apoptosis. Aberrant cellular proliferation, as manifested in cancer, can often be attributed to loss of correct cell cycle control. Inhibition of cdk enzymatic activity therefore provides a means by which abnormally dividing cells can have their division arrested and/or be killed. The diversity of cdks, and cdk complexes, and their critical roles in mediating the cell cycle, provides a broad spectrum of potential therapeutic targets selected on the basis of a defined biochemical rationale.

Progression from the G1 phase to the S phase of the cell cycle is primarily regulated by cdk2, cdk3, cdk4 and cdk6 via association with members of the D and E type cyclins. The D-type cyclins appear instrumental in enabling passage beyond the G1 restriction point, where as the cdk2/cyclin E complex is key to the transition from the G1 to S phase. Subsequent progression through S phase and entry into G2 is thought to require the cdk2/cyclin A complex. Both mitosis, and the G2 to M phase transition which triggers it, are regulated by complexes of cdk1 and the A and B type cyclins.

During G1 phase Retinoblastoma protein (Rb), and related pocket proteins such as p130, are substrates for cdk(2, 4, & 6)/cyclin complexes. Progression through G1 is in part facilitated by hyperphosphorylation, and thus inactivation, of Rb and p130 by the cdk(4/6)/cyclin-D complexes. Hyperphosphorylation of Rb and p130 causes the release of transcription factors, such as E2F, and thus the expression of genes necessary for progression through G1 and for entry into S-phase, such as the gene for cyclin E. Expression of cyclin E facilitates formation of the cdk2/cyclin E complex which amplifies, or maintains, E2F levels via further phosphorylation of Rb. The cdk2/cyclin E complex also phosphorylates other proteins necessary for DNA replication, such as NPAT, which has been implicated in histone biosynthesis. G1 progression and the G1/S transition are also regulated via the mitogen stimulated Myc pathway, which feeds into the cdk2/cyclin E pathway. Cdk2 is also connected to the p53 mediated DNA damage response pathway via p53 regulation of p21 levels. p21 is a protein inhibitor of cdk2/cyclin E and is thus capable of blocking, or delaying, the G1/S transition. The cdk2/cyclin E complex may thus represent a point at which biochemical stimuli from the Rb, Myc and p53 pathways are to some degree integrated. Cdk2 and/or the cdk2/cyclin E complex therefore represent good targets for therapeutics designed at arresting, or recovering control of, the cell cycle in aberrantly dividing cells.

The exact role of cdk3 in the cell cycle is not clear. As yet no cognate cyclin partner has been identified, but a dominant negative form of cdk3 delayed cells in G1, thereby suggesting that cdk3 has a role in regulating the G1/S transition.

Although most cdks have been implicated in regulation of the cell cycle there is evidence that certain members of the cdk family are involved in other biochemical processes. This is exemplified by cdk5 which is necessary for correct neuronal development and which has also been implicated in the phosphorylation of several neuronal proteins such as Tau, NUDE-1, synapsin1, DARPP32 and the Munc18/Syntaxin1A complex. Neuronal cdk5 is conventionally activated by binding to the p35/p39 proteins. Cdk5 activity can, however, be deregulated by the binding of p25, a truncated version of p35. Conversion of p35 to p25, and subsequent deregulation of cdk5 activity, can be induced by ischemia, excitotoxicity, and β-amyloid peptide. Consequently p25 has been implicated in the pathogenesis of neurodegenerative diseases, such as Alzheimer's, and is therefore of interest as a target for therapeutics directed against these diseases.

Cdk7 is a nuclear protein that has cdc2 CAK activity and binds to cyclin H. Cdk7 has been identified as component of the TFIIH transcriptional complex which has RNA polymerase II C-terminal domain (CTD) activity. This has been associated with the regulation of HIV-1 transcription via a Tat-mediated biochemical pathway. Cdk8 binds cyclin C and has been implicated in the phosphorylation of the CTD of RNA polymerase II. Similarly the cdk9/cyclin-T1 complex (P-TEFb complex) has been implicated in elongation control of RNA polymerase II. PTEF-b is also required for activation of transcription of the HIV-1 genome by the viral transactivator Tat through its interaction with cyclin T1. Cdk7, cdk8, cdk9 and the P-TEFb complex are therefore potential targets for anti-viral therapeutics.

At a molecular level mediation of cdk/cyclin complex activity requires a series of stimulatory and inhibitory phosphorylation, or dephosphorylation, events. Cdk phosphorylation is performed by a group of cdk activating kinases (CAKs) and/or kinases such as wee1, Myt1 and Mik1. Dephosphorylation is performed by phosphatases such as cdc25(a & c), pp2a, or KAP.

dilutions of the test compound in DMSO (up to 2.5%). The reaction is allowed to proceed for 3 hours (GSK3-β) before being stopped with an excess of ortho-phosphoric acid (5 μl at 2%). The filtration procedure is as for Activated CDK2/CyclinA assay above.

### Example 9

### Anti-Proliferative Activity

The anti-proliferative activities of the compound of the invention can be determined by measuring the ability of the compound to inhibition of cell growth in a number of cell lines. Inhibition of cell growth is measured using the Alamar Blue assay (Nociari, M. M, Shalev, A., Benias, P., Russo, C. Journal of Immunological Methods 1998, 213, 157-167). The method is based on the ability of viable cells to reduce resazurin to its fluorescent product resorufin. For each proliferation assay cells are plated onto 96 well plates and allowed to recover for 16 hours prior to the addition of inhibitor compounds for a further 72 hours. At the end of the incubation period 10% (v/v) Alamar Blue is added and incubated for a further 6 hours prior to determination of fluorescent product at 535nM ex/590nM em. In the case of the non-proliferating cell assay cells are maintained at confluence for 96 hour prior to the addition of inhibitor compounds for a further 72 hours. The number of viable cells is determined by Alamar Blue assay as before. Cell lines can be obtained from the ECACC (European Collection of cell Cultures).

In particular, the compound of the invention was tested against the HCT-116 cell line (ECACC Reference: 91091005) derived from human colon carcinoma and was found to have an IC₅₀ value of less than 1 μM.

### Example 10

### Determination of Oral Bioavailability

The oral bioavailability of the compound of formula (I) may be determined as follows.

Cdk/cyclin complex activity may be further regulated by two families of endogenous cellular proteinaceous inhibitors: the Kip/Cip family, or the INK family. The INK proteins specifically bind cdk4 and cdk6. p16^{ink4} (also known as MTS1) is a potential tumour suppressor gene that is mutated, or deleted, in a large nunber of primary cancers. The Kip/Cip family contains proteins such as p21^{Cip1,Waf1}, p27^{Kip1} and p57^{kip2}. As discussed previously p21 is induced by p53 and is able to inactivate the cdk2/cyclin(E/A) and cdk4/cyclin(D1/D2/D3) complexes. Atypically low levels of p27 expression have been observed in breast, colon and prostate cancers. Conversely over expression of cyclin E in solid tumours has been shown to correlate with poor patient prognosis. Over expression of cyclin D1 has been associated with oesophageal, breast, squamous, and non-small cell lung carcinomas.

The pivotal roles of cdks, and their associated proteins, in co-ordinating and driving the cell cycle in proliferating cells have been outlined above. Some of the biochemical pathways in which cdks play a key role have also been described. The development of monotherapies for the treatment of proliferative disorders, such as cancers, using therapeutics targeted generically at cdks, or at specific cdks, is therefore potentially highly desirable. Cdk inhibitors could conceivably also be used to treat other conditions such as viral infections, autoimmune diseases and neuro-degenerative diseases, amongst others. Cdk targeted therapeutics may also provide clinical benefits in the treatment of the previously described diseases when used in combination therapy with either existing, or new, therapeutic agents. Cdk targeted anticancer therapies could potentially have advantages over many current antitumour agents as they would not directly interact with DNA and should therefore reduce the risk of secondary tumour development.

Glycogen Synthase Kinase

Glycogen Synthase Kinase-3 (GSK3) is a serine-threonine kinase that occurs as two ubiquitously expressed isoforms in humans (GSK3α & beta GSK3β). GSK3 has been implicated as having roles in embryonic development, protein synthesis, cell proliferation, cell differentiation, microtubule dynamics, cell motility and cellular apoptosis. As such GSK3 has been implicated in the progression of disease states such as diabetes, cancer, Alzheimer's disease, stroke, epilepsy, motor neuron disease and/or head trauma. Phylogenetically GSK3 is most closely related to the cyclin dependent kinases (CDKs).

The consensus peptide substrate sequence recognised by GSK3 is (Ser/Thr)-X-X-X-(pSer/pThr), where X is any amino acid (at positions (n+1), (n+2), (n+3)) and pSer and pThr are phospho-serine and phospho-threonine respectively (n+4). GSK3 phosphorylates the first serine, or threonine, at position (n). Phospho-serine, or phospho-threonine, at the (n+4) position appears necessary for priming GSK3 to give maximal substrate turnover. Phosphorylation of GSK3α at Ser21, or GSK3β at Ser9, leads to inhibition of GSK3. Mutagenesis and peptide competition studies have led to the model that the phosphorylated N-terminus of GSK3 is able to compete with phospho-peptide substrate (S/TXXXpS/pT) via an autoinhibitory mechanism. There are also data suggesting that GSK3α and GSKβ may be subtly regulated by phosphorylation of tyrosines 279 and 216 respectively. Mutation of these residues to a Phe caused a reduction in in vivo kinase activity. The X-ray crystallographic structure of GSK3β has helped to shed light on all aspects of GSK3 activation and regulation.

GSK3 forms part of the mammalian insulin response pathway and is able to phosphorylate, and thereby inactivate, glycogen synthase. Upregulation of glycogen synthase activity, and thereby glycogen synthesis, through inhibition of GSK3, has thus been considered a potential means of combating type II, or non-insulin-dependent diabetes mellitus (NIDDM): a condition in which body tissues become resistant to insulin stimulation. The cellular insulin response in liver, adipose, or muscle tissues is triggered by insulin binding to an extracellular insulin receptor. This causes the phosphorylation, and subsequent recruitment to the plasma membrane, of the insulin receptor substrate (IRS) proteins. Further phosphorylation of the IRS proteins initiates recruitment of phosphoinositide-3 kinase (PI3K) to the plasma membrane where it is able to liberate the second messenger phosphatidylinosityl 3,4,5-trisphosphate (PIP3). This facilitates co-localisation of 3-phosphoinositide-dedependent protein kinase 1 (PDK1) and protein kinase B (PKB or Akt) to the membrane, where PDK1 activates PKB. PKB is able to phosphorylate, and thereby inhibit, GSK3α and/or GSKβ through phosphorylation of Ser9, or ser21, respectively. The inhibition of GSK3 then triggers upregulation of glycogen synthase activity. Therapeutic agents able to inhibit GSK3 may thus be able to induce cellular responses akin to those seen on insulin stimulation. A further in vivo substrate of GSK3 is the eukaryotic protein synthesis initiation factor 2B (eIF2B). eIF2B is inactivated via phosphorylation and is thus able to suppress protein biosynthesis. Inhibition of GSK3, e.g. by inactivation of the “mammalian target of rapamycin” protein (mTOR), can thus upregulate protein biosynthesis. Finally there is some evidence for regulation of GSK3 activity via the mitogen activated protein kinase (MAPK) pathway through phosphorylation of GSK3 by kinases such as mitogen activated protein kinase activated protein kinase 1 (MAPKAP-K1 or RSK). These data suggest that GSK3 activity may be modulated by mitogenic, insulin and/or amino acid stimulii.

It has also been shown that GSK3β is a key component in the vertebrate Wnt signalling pathway. This biochemical pathway has been shown to be critical for normal embryonic development and regulates cell proliferation in normal tissues. GSK3 becomes inhibited in response to Wnt stimulii. This can lead to the de-phosphorylation of GSK3 substrates such as Axin, the adenomatous polyposis coli (APC) gene product and β-catenin. Aberrant regulation of the Wnt pathway has been associated with many cancers. Mutations in APC, and/or β-catenin, are common in colorectal cancer and other tumours. β-catenin has also been shown to be of importance in cell adhesion. Thus GSK3 may also modulate cellular adhesion processes to some degree. Apart from the biochemical pathways already described there are also data implicating GSK3 in the regulation of cell division via phosphorylation of cyclin-D1, in the phosphorylation of transcription factors such as c-Jun, CCAAT/enhancer binding protein a (C/EBPα), c-Myc and/or other substrates such as Nuclear Factor of Activated T-cells (NFATc), Heat Shock Factor-1 (HSF-1) and the c-AMP response element binding protein (CREB). GSK3 also appears to play a role, albeit tissue specific, in regulating cellular apoptosis.

The role of GSK3 in modulating cellular apoptosis, via a pro-apoptotic mechanism, may be of particular relevance to medical conditions in which neuronal apoptosis can occur. Examples of these are head trauma, stroke, epilepsy, Alzheimer's and motor neuron diseases, progressive supranuclear palsy, corticobasal degeneration, and Pick's disease. In vitro it has been shown that GSK3 is able to hyper-phosphorylate the microtubule associated protein Tau. Hyperphosphorylation of Tau disrupts its normal binding to microtubules and may also lead to the formation of intra-cellular Tau filaments. It is believed that the progressive accumulation of these filaments leads to eventual neuronal dysfunction and degeneration. Inhibition of Tau phosphorylation, through inhibition of GSK3, may thus provide a means of limiting and/or preventing neurodegenerative effects.

Diffuse Large B-cell Lymphomas (DLBCL)

Cell cycle progression is regulated by the combined action of cyclins, cyclin-dependent kinases (CDKs), and CDK-inhibitors (CDKi), which are negative cell cycle regulators. p27KIP1 is a CDKi key in cell cycle regulation, whose degradation is required for G1/S transition. In spite of the absence of p27KIP1 expression in proliferating lymphocytes, some aggressive B-cell lymphomas have been reported to show an anomalous p27KIP1 staining. An abnormally high expression of p27KIP1 was found in lymphomas of this type. Analysis of the clinical relevance of these findings showed that a high level of p27KIP1 expression in this type of tumour is an adverse prognostic marker, in both univariate and multivariate analysis. These results show that there is abnormal p27KIP1 expression in Diffuse Large B-cell Lymphomas (DLBCL), with adverse clinical significance, suggesting that this anomalous p27KIP1 protein may be rendered non-functional through interaction with other cell cycle regulator proteins. (Br. J. Cancer. 1999 July;80(9):1427-34. p27KIP1 is abnormally expressed in Diffuse Large B-cell Lymphomas and is associated with an adverse clinical outcome. Saez A, Sanchez E, Sanchez-Beato M, Cruz M A, Chacon I, Munoz E, Camacho F I, Martinez-Montero J C, Mollejo M, Garcia J F, Piris M A. Department of Pathology, Virgen de la Salud Hospital, Toledo, Spain.)

Chronic Lymphocytic Leukemia

B-Cell chronic lymphocytic leukaemia (CLL) is the most common leukaemia in the Western hemisphere, with approximately 10,000 new cases diagnosed each year (Parker S L, Tong T, Bolden S, Wingo P A: Cancer statistics, 1997. Ca. Cancer. J. Clin. 47:5, (1997)). Relative to other forms of leukaemia, the overall prognosis of CLL is good, with even the most advanced stage patients having a median survival of 3 years.

The addition of fludarabine as initial therapy for symptomatic CLL patients has led to a higher rate of complete responses (27%ν3%) and duration of progression-free survival (33ν17 months) as compared with previously used alkylator-based therapies. Although attaining a complete clinical response after therapy is the initial step toward improving survival in CLL, the majority of patients either do not attain complete remission or fail to respond to fludarabine. Furthermore, all patients with CLL treated with fludarabine eventually relapse, making its role as a single agent purely palliative (Rai K R, Peterson B, Elias L, Shepherd L, Hines J, Nelson D, Cheson B, Kolitz J, Schiffer C A: A randomized comparison of fludarabine and chlorambucil for patients with previously untreated chronic lymphocytic leukemia. A CALGB SWOG, CTG/NCI-C and ECOG Inter-Group Study. Blood 88:141a, 1996 (abstr 552, suppl 1). Therefore, identifying new agents with novel mechanisms of action that complement fludarabine's cytotoxicity and abrogate the resistance induced by intrinsic CLL drug-resistance factors will be necessary if further advances in the therapy of this disease are to be realized.

The most extensively studied, uniformly predictive factor for poor response to therapy and inferior survival in CLL patients is aberrant p53 function, as characterized by point mutations or chromosome 17p13 deletions. Indeed, virtually no responses to either alkylator or purine analog therapy have been documented in multiple single institution case series for those CLL patients with abnormal p53 function. Introduction of a therapeutic agent that has the ability to overcome the drug resistance associated with p53 mutation in CLL would potentially be a major advance for the treatment of the disease.

Flavopiridol and CYC 202, inhibitors of cyclin-dependent kinases induce in vitro apoptosis of malignant cells from B-cell chronic lymphocytic leukemia (B-CLL).

Flavopiridol exposure results in the stimulation of caspase 3 activity and in caspase-dependent cleavage of p27(kip1), a negative regulator of the cell cycle, which is overexpressed in B-CLL (Blood. 1998 Nov. 15;92(10):3804-16 Flavopiridol induces apoptosis in chronic lymphocytic leukemia cells via activation of caspase-3 without evidence of bcl-2 modulation or dependence on functional p53. Byrd J C, Shinn C, Waselenko J K, Fuchs E J, Lehman T A, Nguyen P L, Flinn I W, Diehl L F, Sausville E, Grever M R).

WO 02/34721 from Du Pont discloses a class of indeno [1,2-c]pyrazol-4-ones as inhibitors of cyclin dependent kinases.

WO 01/81348 from Bristol Myers Squibb describes the use of 5-thio-, sulphinyl- and sulphonylpyrazolo[3,4-b]-pyridines as cyclin dependent kinase inhibitors.

WO 00/62778 also from Bristol Myers Squibb discloses a class of protein tyrosine kinase inhibitors.

WO 01/72745A1 from Cyclacel describes 2-substituted 4-heteroaryl-pyrimidines and their preparation, pharmaceutical compositions containing them and their use as inhibitors of cyclin-dependant kinases (CDKs) and hence their use in the treatment of proliferative disorders such as cancer, leukaemia, psoriasis and the like.

WO 99/21845 from Agouron describes 4-aminothiazole derivatives for inhibiting cyclin-dependent kinases (CDKs), such as CDK1, CDK2, CDK4, and CDK6. The invention is also directed to the therapeutic or prophylactic use of pharmaceutical compositions containing such compounds and to methods of treating malignancies and other disorders by administering effective amounts of such compounds.

WO 01/53274 from Agouron discloses as CDK kinase inhibitors a class of compounds which can comprise an amide-substituted benzene ring linked to an N-containing heterocyclic group.

WO 01/98290 (Pharmacia & Upjohn) discloses a class of 3-aminocarbonyl-2-carboxamido thiophene derivatives as protein kinase inhibitors.

WO 01/53268 and WO 01/02369 from Agouron disclose compounds that mediate or inhibit cell proliferation through the inhibition of protein kinases such as cyclin dependent kinase or tyrosine kinase. The Agouron compounds have an aryl or heteroaryl ring attached directly or though a CH═CH or CH═N group to the 3-position of an indazole ring.

WO 00/39108 and WO 02/00651 (both to Du Pont Pharmaceuticals) describe heterocyclic compounds that are inhibitors of trypsin-like serine protease enzymes, especially factor Xa and thrombin. The compounds are stated to be useful as anticoagulants or for the prevention of thromboembolic disorders.

US 2002/0091116 (Zhu et al.), WO 01/19798 and WO 01/64642 each disclose diverse groups of heterocyclic compounds as inhibitors of Factor Xa. Some 1-substituted pyrazole carboxamides are disclosed and exemplified.

U.S. Pat. No. 6,127,382, WO 01/70668, WO 00/68191, WO 97/48672, WO 97/19052 and WO 97/19062 (all to Allergan) each describe compounds having retinoid-like activity for use in the treatment of various hyperproliferative diseases including cancers.

WO 02/070510 (Bayer) describes a class of amino-dicarboxylic acid compounds for use in the treatment of cardiovascular diseases. Although pyrazoles are mentioned generically, there are no specific examples of pyrazoles in this document.

WO 97/03071 (Knoll AG) discloses a class of heterocyclyl-carboxamide derivatives for use in the treatment of central nervous system disorders. Pyrazoles are mentioned generally as examples of heterocyclic groups but no specific pyrazole compounds are disclosed or exemplified.

WO 97/40017 (Novo Nordisk) describes compounds that are modulators of protein tyrosine phosphatases.

WO 03/020217 (Univ. Connecticut) discloses a class of pyrazole 3-carboxamides as cannabinoid receptor modulators for treating neurological conditions. It is stated (page 15) that the compounds can be used in cancer chemotherapy but it is not made clear whether the compounds are active as anti-cancer agents or whether they are administered for other purposes.

WO 01/58869 (Bristol Myers Squibb) discloses cannabinoid receptor modulators that can be used inter alia to treat a variety of diseases. The main use is the treatment of respiratory diseases, although reference is made to the treatment of cancer.

WO 01/02385 (Aventis Crop Science) discloses 1-(quinoline-4-yl)-1H-pyrazole derivatives as fungicides. 1-Unsubstituted pyrazoles are disclosed as synthetic intermediates.

WO 2004/039795 (Fujisawa) discloses amides containing a 1-substituted pyrazole group as inhibitors of apolipoprotein B secretion. The compounds are stated to be useful in treating such conditions as hyperlipidemia.

WO 2004/000318 (Cellular Genomics) discloses various amino-substituted monocycles as kinase modulators. None of the exemplified compounds are pyrazoles.

WO 2005/012256 (Astex Technology Limited) discloses the compound 4-(2,6-dichloro-benzoylamino)-1H-pyrazole-3-carboxylic acid piperidin-4-ylamide and analogues thereof as being inhibitors of Cyclin Dependent Kinases (CDK kinases) and Glycogen Synthase Kinase-3 (GSK3).

The compound 4-(2,6-dichloro-benzoylamino)-1H-pyrazole-3-carboxylic acid (1-methanesulphonyl-piperidin-4-yl)-amide is disclosed in our earlier International patent application number PCT/GB2006/000193 (the contents of which are incorporated herein by reference) as being an inhibitor of Cyclin Dependent Kinases (CDK kinases) and Glycogen Synthase Kinase-3 (GSK3). The preparation of the compound is described in Example 1 of PCT/GB2006/000193 and the final step in Example 1 involves the isolation of the compound from an ethyl acetate solution by evaporation of the solvent under reduced pressure. It is believed that 4-(2,6-dichloro-benzoylamino)-1H-pyrazole-3-carboxylic acid (1-methanesulphonyl-piperidin-4-yl)-amide produced by this method is amorphous.

### SUMMARY OF THE INVENTION

Crystalline Forms

In a first aspect, the present invention provides 4-(2,6-dichloro-benzoylamino)-1H-pyrazole-3-carboxylic acid (1-methanesulphonyl-piperidin-4-yl)-amide in a substantially crystalline form.

The compound 4-(2,6-dichloro-benzoylamino)-1H-pyrazole-3-carboxylic acid (1-methanesulphonyl-piperidin-4-yl)-amide has the formula (I):

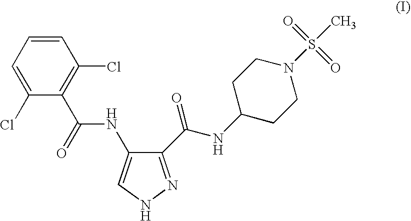

or a tautomeric form thereof. The compound of the formula (I) may be referred to in this application by its chemical name or, for convenience, as “the compound”, “the compound of formula (I)” or “the compound of the invention”. Each of these synonyms refers to the compound shown in formula (I) above and having the chemical name 4-(2,6-dichloro-benzoylamino)-1H-pyrazole-3-carboxylic acid (1-methanesulphonyl-piperidin-4-yl)-amide.

Although the compound of formula (I) can form salts with the basic nitrogen atom in the pyrazole ring, references to the compound in substantially crystalline form are references to the free base.

References to the compound 4-(2,6-dichloro-benzoylamino)-1H-pyrazole-3-carboxylic acid (1-methanesulphonyl-piperidin-4-yl)-amide, where the context admits, include within their scope all solvates, tautomers and isotopes thereof.

Compounds of the formula (I) may exist in a number of different geometric isomeric, and tautomeric forms and references to compounds of the formula (I) include all such forms. For the avoidance of doubt, where a compound can exist in one of several geometric isomeric or tautomeric forms and only one is specifically described or shown, all others are nevertheless embraced by formula (I).

For example, in the compound of the formula (I) the pyrazole ring can exist in the two tautomeric forms A and B below. For simplicity, the general formula (I) illustrates form A but the formula is to be taken as embracing both tautomeric forms.

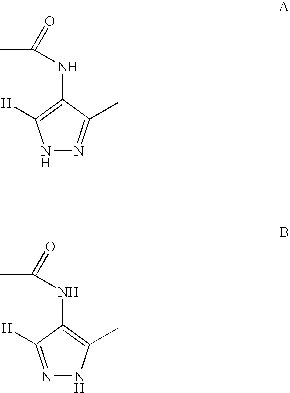

The compound of the invention also includes compounds with one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O.

The isotopes may be radioactive or non-radioactive. In one embodiment of the invention, the compound contains no radioactive isotopes. Such a compound is preferred for therapeutic use. In another embodiment, however, the compound may contain one or more radioisotopes. Compounds containing such radioisotopes may be useful in a diagnostic context.

According to the first aspect of the invention, the compound is substantially crystalline; i.e. it is from 50% to 100% crystalline.

More particularly, the compound may be at least 55% crystalline, or at least 60% crystalline, or at least 65% crystalline, or at least 70% crystalline, or at least 75% crystalline, or at least 80% crystalline, or at least 85% crystalline,or at least 90% crystalline, or at least 95% crystalline, or at least 98% crystalline, or at least 99% crystalline, or at least 99.5% crystalline, or at least 99.9% crystalline, for example 100% crystalline.

The crystalline forms of the compound of the invention may be solvated (e.g. hydrated) or non-solvated (e.g. anhydrous).

The term “anhydrous” as used herein does not exclude the possibility of the presence of some water on or in the compound (e.g. a crystal of the compound). For example, there may be some water present on the surface of the compond (e.g. compound crystal), or minor amounts within the body of the compound (e.g. crystal). Typically, an anhydrous form contains fewer than 0.4 molecules of water per molecule of compound, and more preferably contains fewer than 0.1 molecules of water per molecule of compound, for example 0 molecules of water.

In one embodiment, the compound is anhydrous.

In another embodiment, the compound is solvated, e.g. hydrated. Where the salts are hydrated, they can contain, for example, up to three molecules of water of crystallisation, more usually up to two molecules of water, e.g. one molecule of water or two molecules of water. Non-stoichiometric hydrates may also be formed in which the number of molecules of water present is less than one or is otherwise a non-integer. For example, where there is less than one molecule of water present, there may be for example 0.4, or 0.5, or 0.6, or 0.7, or 0.8, or 0.9 molecules of water present per molecule of compound.

Other solvates include alcoholates such as ethanolates and isopropanolates.

The crystalline forms described herein, crystals thereof and their crystal structure form further aspects of the invention.

The crystals and their crystal structure can be characterised using a number of techniques including single crystal X-ray crystallography, X-ray powder diffraction (XRPD), differential scanning calorimetry (DSC) and infra red spectroscopy, e.g. Fourier Transform infra-red spectroscopy (FTIR). The behaviour of the crystals under conditions of varying humidity can be analysed by gravimetric vapour sorption studies and also by XRPD.

Determination of the crystal structure of a compound can be performed by X-ray crystallography which can be carried out according to conventional methods, such as those described herein and in Fundamentals of Crystallography, C. Giacovazzo, H. L. Monaco, D. Viterbo, F. Scordari, G. Gilli, G. Zanotti and M. Catti, (International Union of Crystallography/Oxford University Press, 1992 ISBN 0-19-855578-4 (p/b), 0-19-85579-2 (h/b)). This technique involves the analysis and interpretation of the X-ray diffraction of a single crystal.

In the substantially crystalline form of 4-(2,6-dichloro-benzoylamino)-1H-pyrazole-3-carboxylic acid (1-methanesulphonyl-piperidin-4-yl)-amide, one single crystalline form may predominate, although other crystalline forms may be present in minor and preferably negligible amounts.

In a preferred embodiment, the invention provides a substantially crystalline form of the compound 4-(2,6-dichloro-benzoylamino)-1H-pyrazole-3-carboxylic acid (1-methanesulphonyl-piperidin-4-yl)-amide containing a single crystalline form of the dehydrate of the compound and no more than 5% by weight of any other crystalline forms of the compound.

Preferably, the single crystalline form is accompanied by less than 4%, or less than 3%, or less than 2% of other crystalline forms, and in particular contains less than or equal to about 1% by weight of other crystalline forms. More preferably, the single crystalline form is accompanied by less than 0.9%, or less than 0.8%, or less than 0.7%, or less than 0.6%, or less than 0.5%, or less than 0.4%, or less than 0.3%, or less than 0.2%, or less than 0.1%, or less than 0.05%, or less than 0.01%, by weight of other crystalline forms, for example 0% by weight of other crystalline forms.

The crystalline forms of 4-(2,6-dichloro-benzoylamino)-1H-pyrazole-3-carboxylic acid (1-methanesulphonyl-piperidin-4-yl)-amide can be prepared by synthesizing the compound using the methods described in PCT/GB2006/000193 or methods described herein, and then subjecting the compound to one or more recrystallisation steps.

The use of the term “recrystallisation” herein does not require the compound to be in a crystalline form before the recrystallisation process. On the contrary, although the starting material for the recrystallisation process can be crystalline or partly crystalline, it may alternatively be in an amorphous form prior to recrystallisation.

The recrystallisation of 4-(2,6-dichloro-benzoylamino)-1H-pyrazole-3-carboxylic acid (1-methanesulphonyl-piperidin-4-yl)-amide can be carried out by methods well known to the skilled person. As is well known, a good recrystallization solvent should dissolve a moderate quantity of the substance to be purified at elevated temperatures but only a small quantity of the substance at lower temperature. It should dissolve impurities readily at low temperatures or not at all. Finally, the solvent should be readily removed from the purified product. This usually means that it has a relatively low boiling point and a person skilled in the art will know recrystallizing solvents for a particular substance or, if that information is not available, will test several solvents until an appropriate solvent or solvent mixture is found. In order to get a good yield of purified material, the minimum amount of hot solvent to dissolve all the impure material is used. In practice, 3-5% more solvent than necessary typically is used so that the solution is not saturated. If the impure compound contains an impurity which is insoluble in the solvent it may then be removed by filtration and then allowing the solution to crystallize. In addition, if the impure compound contains traces of coloured material that are not native to the compound, they may be removed by adding a small amount of decolorizing charcoal to the hot solution, filtering it and then allowing it to crystallize.

Crystallization may occur spontaneously upon cooling the solution. However, if it does not occur spontaneously, then crystallization may be induced by cooling the solution below room temperature or by adding a single crystal of pure material (a seed crystal). Recrystallisation can also be carried out and/or the yield optimized by the use of an anti-solvent. In this case, the compound is dissolved in a suitable solvent at elevated temperature, filtered and then an additional solvent in which the required compound has low solubility is added to aid crystallization. The crystals are then typically isolated using vacuum filtration, washed and then dried, for example, in an oven or via desiccation.

Other examples of methods for crystallization include crystallization from a vapour, which includes an evaporation step, for example in a sealed tube or an air stream, and crystallization from melt (Crystallization Technology Handbook 2nd Edition, edited by A. Mersmann, 2001).

In one embodiment of the invention, the crystalline form of 4-(2,6-dichloro-benzoylamino)-1H-pyrazole-3-carboxylic acid (1-methanesulphonyl-piperidin-4-yl)-amide is prepared by recrystallising the compound using a mixture of N,N-dimethylacetamide, acetone and water.

For example, the 4-(2,6-dichloro-benzoylamino)-1H-pyrazole-3-carboxylic acid (1-methanesulphonyl-piperidin-4-yl)-amide can be recrystallised by a method involving the steps of:

(a) dissolving the compound in a mixture of N,N-dimethylacetamide and acetone (e.g. in a volume ratio of 1.5:2) with heating (e.g. to a temperature of up to about 50° C., for example 40 to 50° C.);

(b) optionally clarifying the solution where required by filtration;

(c) adding water whilst maintaining or increasing the heating (e.g. to a temperature of 60 to 80° C.);

(d) cooling the solution, or allowing the solution to cool, to enable crystallisation to take place; and

(e) isolating the crystalline form of the compound, for example by filtration.

Crystals of 4-(2,6-dichloro-benzoylamino)-1H-pyrazole-3-carboxylic acid (1-methanesulphonyl-piperidin-4-yl)-amide prepared using the N,N-dimethylacetamide/acetone/water solvent system have been subjected to characterisation by X-ray crystallography.

Table 1 gives coordinate data for crystals of 4-(2,6-dichloro-benzoylamino)-1H-pyrazole-3-carboxylic acid (1-methanesulphonyl-piperidin-4-yl)-amide in Crystallographic Information File (CIF) Format (see Hall, Allen and Brown, Acta Cryst. (1991). A47, 655-685; http://www.iucr.ac.uk/iucr-top/cif/home.html). Alternative file formats such as a PDB file format (e.g. format consistent with that of the EBI Macromolecular Structure Database (Hinxton, UK)) may be used or preferred by others of skill in the art. However it will be apparent that the use of a different file format to present or manipulate the coordinates of the Tables is within the scope of the present invention. The crystal structure of the compound is illustrated in FIGS. 1 and 2, the thermal ellipsoid representation of the structure generated by the X-ray diffraction study being provided in FIG. 1 and the packing diagram being provided in FIG. 2.

From the X-ray crystallography studies, it has been found that the compound of the invention has a crystal structure that belongs belong to a monoclinic space group such as C2/c (#15) with crystal lattice parameters a=9.15, b=31.32, c=7.93 Å, β=113.3°, α=γ=90°.

Accordingly, in another embodiment, the invention provides a crystalline form of 4-(2,6-dichloro-benzoylamino)- 1H-pyrazole-3-carboxylic acid (1-methanesulphonyl-piperidin-4-yl)-amide which:

(a) has a crystal structure as set out in FIGS. 1 and 2; and/or

(b) has a crystal structure as defined by the coordinates in Table 1 herein; and/or

(c) has crystal lattice parameters at a=9.15, b=31.32, c=7.93 Å, β=113.3°, α=γ=90°; and/or

(d) has a crystal structure that belongs belong to a monoclinic space group such as C2/c (#15).

Alternatively, or additionally, the crystalline structure of the crystalline compound of the invention can be analysed by the solid state technique of X-ray Powder Diffraction (XRPD). XRPD can be carried out according to conventional methods such as those described herein (see the examples) and in Introduction to X-ray Powder Diffraction, Ron Jenkins and Robert L. Snyder (John Wiley & Sons, New York, 1996). The presence of defined peaks (as opposed to random background noise) in an XRPD diffractogram indicates that the compound has a degree of crystallinity.

A compound's X-ray powder pattern is characterised by the diffraction angle (2θ) and interplanar spacing (d) parameters of an X-ray diffraction spectrum. These are related by Bragg's equation, nλ=2d Sin θ, (where n=1; λ=wavelength of the cathode used; d=interplanar spacing; and θ=diffraction angle). Herein, interplanar spacings, diffraction angle and overall pattern are important for identification of crystal in the X-ray powder diffraction, due to the characteristics of the data. The relative intensity should not be strictly interpreted since it may be varied depending on the direction of crystal growth, particle sizes and measurement conditions. In addition, the diffraction angles usually mean ones which coincide in the range of 2θ±0.2°. The peaks mean main peaks and include peaks not larger than medium at diffraction angles other than those stated above.

The crystalline form of the compound 4-(2,6-dichloro-benzoylamino)-1H-pyrazole-3-carboxylic acid (1-methanesulphonyl-piperidin-4-yl)-amide prepared using the N,N-dimethylacetamide/acetone/water solvent system has been characterised by XRPD and has an X-ray powder diffraction pattern essentially as shown in FIG. 3.

The powder X-ray diffraction patterns are expressed in terms of the diffraction angle (2θ), inter planar spacing (d) and relative intensities.

Accordingly, in another embodiment, the invention provides a substantially crystalline form of 4-(2,6-dichloro-benzoylamino)-1H-pyrazole-3-carboxylic acid (1-methanesulphonyl-piperidin-4-yl)-amide having an X-ray powder diffraction pattern characterised by the presence of major peaks at the diffraction angles (2θ) and interplanar spacings (d) set forth in Table A.

TABLE A

| 2θ/° | d/Å | I |
|---|---|---|
| 16.57 | 5.35 | 59 |
| 16.95 | 5.23 | 62 |
| 20.42 | 4.35 | 76 |
| 22.66 | 3.92 | 100 |
| 24.33 | 3.66 | 40 |

The X-ray powder diffraction pattern is preferably further characterised by the presence of additional peaks at the diffraction angles (2θ) and interplanar spacings (d) set forth in Table B.

TABLE B

| 2θ/° | d/Å | I |
|---|---|---|
| 5.63 | 15.70 | 24 |
| 12.56 | 7.05 | 26 |
| 13.35 | 6.63 | 27 |
| 14.89 | 5.95 | 18 |
| 19.53 | 4.55 | 37 |
| 20.88 | 4.25 | 23 |
| 24.99 | 3.56 | 16 |

The invention further provides a substantially crystalline form of 4-(2,6-dichloro-benzoylamino)-1H-pyrazole-3-carboxylic acid (1-methanesulphonyl-piperidin-4-yl)-amide which exhibits peaks at the same diffraction angles as those of the X-ray powder diffraction pattern shown in FIG. 3. Preferably the peaks have the same relative intensity as the peaks in FIG. 3.

In a preferred embodiment, the invention provides a substantially crystalline form of 4-(2,6-dichloro-benzoylamino)-1H-pyrazole-3-carboxylic acid (1-methanesulphonyl-piperidin-4-yl)-amide having an X-ray powder diffraction pattern substantially as shown in FIG. 3.

The crystalline form of the compound of the invention can also be characterised by differential scanning calorimetry (DSC).

The crystalline form of 4-(2,6-dichloro-benzoylamino)-1H-pyrazole-3-carboxylic acid (1-methanesulphonyl-piperidin-4-yl)-amide prepared using the N,N-dimethylacetamide/acetone/water solvent system has been analysed by DSC and exhibits an endothermic peak at 293-296° C., for example 294.5-295° C., indicative of the thermally induced melting of the crystalline lattice. No significant transitions were apparent prior to the main melting endotherm thus indicating that the crystalline form of the compound of the invention is anhydrous. The DSC scan is shown in FIG. 4.

Accordingly, in another aspect, the invention provides a crystalline form of 4-(2,6-dichloro-benzoylamino)-1H-pyrazole-3-carboxylic acid (1-methanesulphonyl-piperidin-4-yl)-amide which is anhydrous and exhibits an endothermic peak at 293-296° C., for example 294.5-295° C. when subjected to DSC.

The novel crystalline form of the compound of the invention can be further characterised by infra-red spectroscopy, e.g. FTIR.

The infra-red spectrum of the crystalline form of the compound prepared using the N,N-dimethylacetamide/acetone/water solvent system includes characteristic peaks, when analysed using the UATR method, at 3362, 3019, 2843, 1677, 1577, 1547, 1533, 1326, 1150, 926, 781, 667 cm⁻¹.

Without wishing to be bound by any theory, it is believed that the infra red peaks can be assigned to structural components of the salt as follow:

| Peak: | Due to: |
|---|---|
| 3361.92 cm⁻¹ | N—H |
| 3018.97 cm⁻¹ | aromatic C—H |
| 2842.99 cm⁻¹ | aliphatic C—H |
| 1676.72 cm⁻¹ | amide C═O |
| 1577.31, 1546.92, 1532.94 cm⁻¹ | amide |
| 1325.63 cm⁻¹ | aromatic C—N |
| 1149.91 cm⁻¹ | 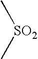 |
| 925.73 cm⁻¹ | C—H aromatic |
| 780.75, 666.88 cm⁻¹ | aromatic C—H |

Accordingly, in a further embodiment, the invention provides a substantially crystalline form of 4-(2,6-dichloro-benzoylamino)-1H-pyrazole-3-carboxylic acid (1-methanesulphonyl-piperidin-4-yl)-amide that exhibits an infra-red spectrum when analysed using the Universal Attenuated Total Reflectance (UATR) method, containing characteristic peaks at 3362, 3019, 2843, 1677, 1577, 1547, 1533, 1326, 1150, 926, 781, 667 cm⁻¹.

As will be evident from the foregoing paragraphs, the novel crystalline form of the compound of the invention can be characterised by a number of different physicochemical parameters. Accordingly, in a preferred embodiment, the invention provides a crystalline form of 4-(2,6-dichloro-benzoylamino)-1H-pyrazole-3-carboxylic acid (1-methanesulphonyl-piperidin-4-yl)-amide which is characterised by any one or more (in any combination) or all of the following parameters, namely that the crystalline form:

(a) has a crystal structure as set out in FIGS. 1 and 2; and/or

(b) has a crystal structure as defined by the coordinates in Table 1 herein; and/or

(c) has crystal lattice parameters at a=9.15, b=31.32, c=7.93 Å, β=113.3°, α=γ=90°; and/or

(d) has a crystal structure that belongs belong to a monoclinic space group such as C2/c (#15); and/or

(e) has an X-ray powder diffraction pattern characterised by the presence of major peaks at the diffraction angles (2θ) and interplanar spacings (d) set forth in Table A, and optionally Table B; and/or

(f) exhibits peaks at the same diffraction angles as those of the X-ray powder diffraction pattern shown in FIG. 3 and optionally wherein the peaks have the same relative intensity as the peaks in FIG. 3; and/or

(g) has an X-ray powder diffraction pattern substantially as shown in FIG. 3; and/or

(h) is anhydrous and exhibits an endothermic peak at 293-296° C., for example 294.5-295° C., when subjected to DSC; and/or

(i) exhibits an infra-red spectrum, when analysed using the Universal Attenuated Total Reflectance (UATR) method, that contains characteristic peaks at containing characteristic peaks at 3362, 3019, 2843, 1677, 1577, 1547, 1533, 1326, 1150, 926, 781, 667 cm⁻¹.

Processes for Preparing 4-(2,6-dichloro-benzoylamino)-1H-pyrazole-3-carboxylic acid (1-methanesulphonyl-piperidin-4-yl)-amide

In Example 1 of our earlier application PCT/GB2006/00, it is disclosed that 4-(2,6-dichloro-benzoylamino)-1H-pyrazole-3-carboxylic acid (1-methanesulphonyl-piperidin-4-yl)-amide can be prepared by a sequence of steps including:

(i) reacting 4-(2,6-dichloro-benzoylamino)-1H-pyrazole-3-carboxylic acid with 4-amino-1-tert-butyloxycarbonyl-piperidine in the presence of 1-ethyl-3-(3′-dimethylaminopropyl)-carbodiimide (EDC) and 1-hydroxybenzotriazole (HOBt) in dimethyl formamide (DMF) to give the N-boc protected form of 4-(2,6-dichloro-benzoylamino)-1H-pyrazole-3-carboxylic acid piperidin-4-ylamide;

(ii) removing the boc protecting group by treatment with hydrochloric acid; and

(iii) reacting the 4-(2,6-dichloro-benzoylamino)-1H-pyrazole-3-carboxylic acid piperidin-4-ylamide hydrochloride in acetonitrile, and in the presence of diisopropylethylamine, with methanesulphonyl chloride.

It has now been found that instead of using a tertiary amine as the base in step (iii), the mesylation step can be carried out using a metal carbonate or bicarbonate as the base.

Accordingly, in another aspect, the invention provides a process for preparing 4-(2,6-dichloro-benzoylamino)-1H-pyrazole-3-carboxylic acid (1-methanesulphonyl-piperidin-4-yl)-amide, which process comprises the reaction of a compound of the formula (II):

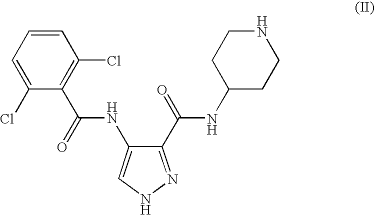

with methanesulphonyl chloride in a polar solvent in the presence of a base selected from alkali metal carbonates and bicarbonates; and thereafter isolating and optionally recrystallising the 4-(2,6-dichloro-benzoylamino)-1H-pyrazole-3-carboxylic acid (1-methanesulphonyl-piperidin-4-yl)-amide thus formed.

The base is preferably an alkali metal bicarbonate such as sodium bicarbonate.

The polar solvent can be water or a mixture of water and an organic solvent, preferably a polar solvent such as ethyl acetate.

The reaction with methanesulphonyl chloride may be carried out at a temperature of 0° C. up to about 30° C., more typically about 12° C. up to about 28° C., e.g. 15° C. to 25° C.

The compound of formula (II) may initially be present in the reaction mixture as a methanesulphonate salt which can be formed by deprotection of the N-tert-butoxycarbonyl (boc) protected compound (III).

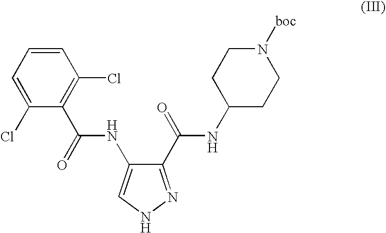

In order to mimimise or avoid the presence of significant amounts of the boc-protected intermediate (III) in the final product, the compound of formula (II) may be treated with methanesulphonic acid and heated to a temperature of 50° C. or more (e.g. 80° C. or more, or 90° C. or more, for example 95° C. to 105° C.) prior to cooling and reacting with the methanesulphonyl choride.

Accordingly, in a further aspect the invention provides a process for the preparation of 4-(2,6-dichloro-benzoylamino)-1H-pyrazole-3-carboxylic acid (1-methanesulphonyl-piperidin-4-yl)-amide, which process comprises:

(a) reacting a compound of the formula (III) with methanesulphonic acid in a polar solvent (e.g. dioxane) to remove the boc group and give a methanesulphonate salt of a compound of the formula (II)

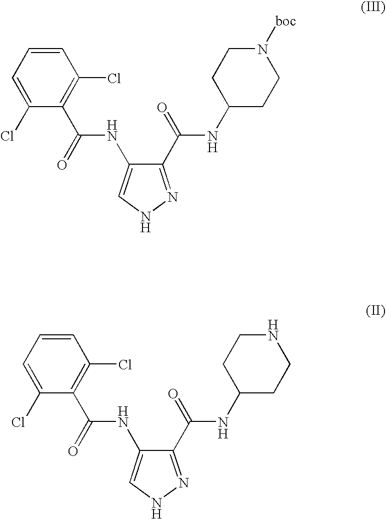

(b) isolating the methanesulphonate salt of the compound of formula (II);

(c) treating the methanesulphonate salt of the compound of formula (II) with methanesulphonic acid in an polar solvent (e.g. an aqueous solvent such as water) to convert remaining traces of compound (III) to compound (II); and

(d) reacting the product of step (c) with methanesulphonyl chloride in a polar solvent in the presence of a base selected from alkali metal carbonates and bicarbonates; and thereafter isolating and optionally recrystallising the 4-(2,6-dichloro-benzoylamino)-1H-pyrazole-3-carboxylic acid (1-methanesulphonyl-piperidin-4-yl)-amide thus formed.

The compound of formula (III) can be prepared according to the methods described in Example 237 of our earlier application PCT/GB2004/003179 (WO 2005/012256) or the methods described in Example 1 of our earlier application PCT/GB2006/000193, and as described in the examples herein.

In Example 1 of PCT/GB2006/000193, compound (III) is formed by reacting 4-(2,6-dichloro-benzoylamino)-1H-pyrazole-3-carboxylic acid with 4-amino-1-tert-butyloxycarbonyl-piperidine in the presence of 1-ethyl-3-(3′-dimethylaminopropyl)-carbodiimide (EDC) and 1-hydroxybenzotriazole (HOBt) in dimethyl formamide (DMF).

It has now been found that instead of using EDC and HOBt to activate the carboxylic acid and promote formation of the amide bond, 4-amino-1-tert-butyloxycarbonyl-piperidine can instead be reacted with the acid chloride of 4-(2,6-dichloro-benzoylamino)-1H-pyrazole-3-carboxylic acid.

Accordingly, in another aspect, the invention provides a process for the preparation of 4-(2,6-dichloro-benzoylamino)-1H-pyrazole-3-carboxylic acid (1-methanesulphonyl-piperidin-4-yl)-amide, which process comprises:

(ia) reacting an acid chloride compound of the formula (IV) with a compound of the formula (V):

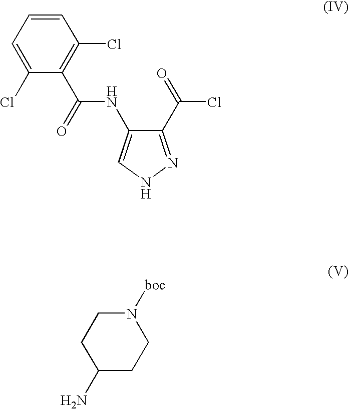

in a polar solvent in the presence of a base (e.g. a non-interfering base such as a tertiary amine—for example triethylamine) to give a compound of the formula (III):

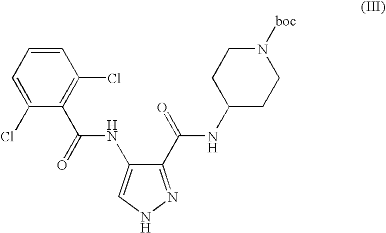

(a) reacting a compound of the formula (III) with methanesulphonic acid in a polar solvent (e.g. dioxane) to remove the boc group and give a methanesulphonate salt of a compound of the formula (II)

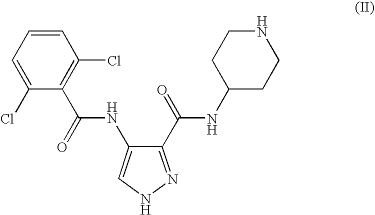

(b) isolating the methanesulphonate salt of the compound of formula (II);

(c) treating the methanesulphonate salt of the compound of formula (II) with methanesulphonic acid in an polar solvent (e.g. an aqueous solvent such as water) to convert remaining traces of compound (III) to compound (II); and

(d) reacting the product of step (c) with methanesulphonyl chloride in a polar solvent in the presence of a base selected from alkali metal carbonates and bicarbonates; and thereafter isolating and optionally recrystallising the 4-(2,6-dichloro-benzoylamino)-1H-pyrazole-3-carboxylic acid (1-methanesulphonyl-piperidin-4-yl)-amide thus formed.

The acid chloride (IV) can be made according to methods well known to the skilled person, for example by treatment of the carboxylic acid with thionyl chloride, or by reaction with oxalyl chloride in the presence of a catalytic amount of dimethyl formamide, or by reaction of a potassium salt of the acid with oxalyl chloride. When thionyl chloride is used to generate the acid chloride, the reaction with the carboxylic acid is typically carried out with heating to a temperature in excess of 50° C., for example 80 to 100° C., in the presence of an inert solvent such as toluene.

An illustrative synthetic route for preparing a compound of the formula (I) is shown in Scheme 1.

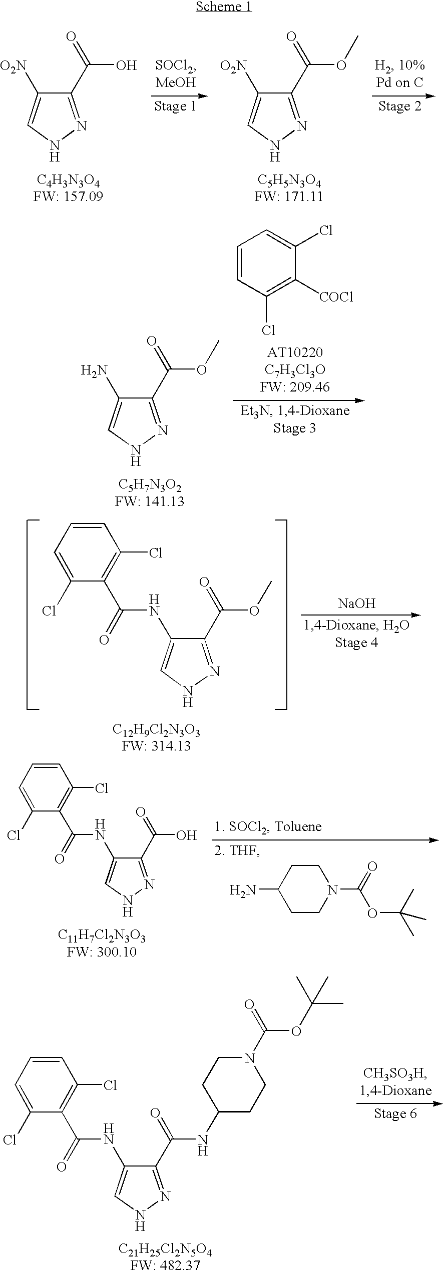

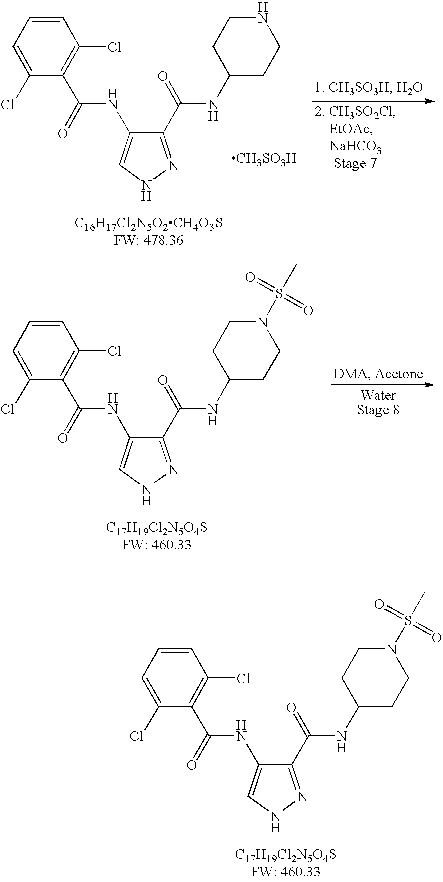

In another aspect, the invention provides a process for the preparation of 4-(2,6-dichloro-benzoylamino)-1H-pyrazole-3-carboxylic acid (1-methanesulphonyl-piperidin-4-yl)-amide, which process comprises the reaction of a compound of the formula (VI) with 2,6-dichlorobenzoic acid or an activated derivative thereof such as 2,6-dichlorobenzoyl chloride.

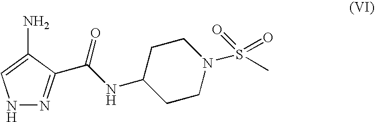

The reaction with the acid chloride is typically carried out in the presence of a base, for example a non-interefering base such as a tertiary amine (e.g. triethylamine). The reaction is usually carried out in the presence of a solvent, for example a halogenated solvent such as dichloromethane, or an aromatic hydrocarbon solvent such as toluene or a polar aprotic solvent such as dioxane, optionally with mild heating, for example to a temperature of up to about 60° C., e.g. up to about 45° C.

Where the compound of formula (VI) is reacted with 2,6-dichlorobenzoic acid, the amide bond formation may be brought about by the use of amide coupling reagents of the type commonly used in the formation of peptide linkages. Examples of such reagents include 1,3-dicyclohexylcarbodiimide (DCC) (Sheehan et al, J. Amer. Chem Soc. 1955, 77, 1067), 1-ethyl-3-(3′-dimethylaminopropyl)-carbodiimide (referred to herein either as EDC or EDAC but also known in the art as EDCI and WSCDI) (Sheehan et al, J. Org. Chem., 1961, 26, 2525), uronium-based coupling agents such as O-(7-azabenzotriazol-1-yl)-N,N,N′,N′-tetramethyluronium hexafluorophosphate (HATU) and phosphonium-based coupling agents such as 1-benzo-triazolyloxytris-(pyrrolidino)phosphonium hexafluorophosphate (PyBOP) (Castro et al, Tetrahedron Letters, 1990, 31, 205). Carbodiimide-based coupling agents are advantageously used in combination with 1-hydroxy-7-azabenzotriazole (HOAt) (L. A. Carpino, J. Amer. Chem. Soc., 1993, 115, 4397) or 1-hydroxybenzotriazole (HOBt) (Konig et al, Chem. Ber., 103, 708, 2024-2034). Preferred coupling reagents include EDC (EDAC) and DCC in combination with HOAt or HOBt.

The coupling reaction is typically carried out in a non-aqueous, non-protic solvent such as acetonitrile, dioxan, dimethylsulphoxide, dichloromethane, dimethylformamide or N-methylpyrrolidine, or in an aqueous solvent optionally together with one or more miscible co-solvents. The reaction can be carried out at room temperature or at an appropriately elevated temperature. The reaction may be carried out in the presence of a non-interfering base, for example a tertiary amine such as triethylamine or N,N-diisopropylethylamine.

A synthetic route for preparing a compound of formula (I) by a process involving an intermediate of the formula (VI) is illustrated in Scheme 2.

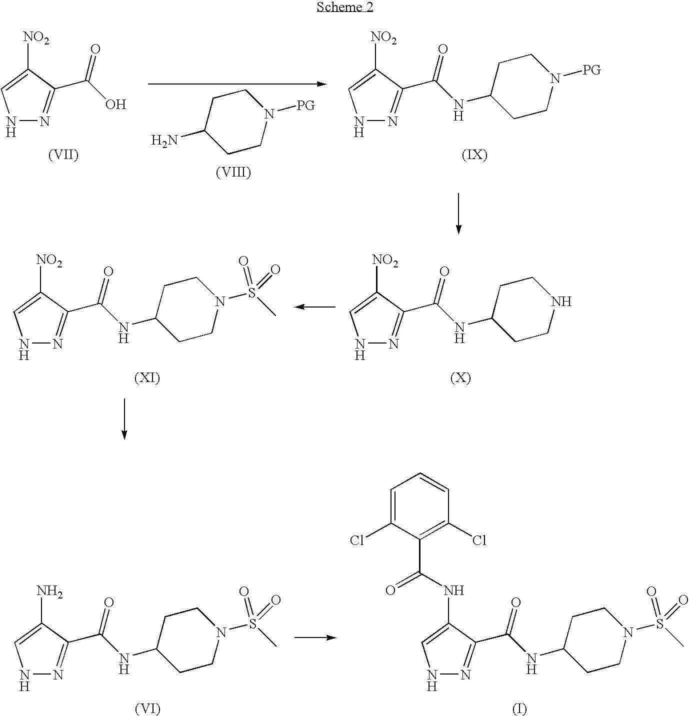

In Scheme 2, the 4-nitropyrazole carboxylic acid (VII) is coupled with the protected piperidine amine (VIII) using standard methods, for example by forming an acid chloride which then reacts with the amine (VIII) or by using an amide coupling agent of the type described above, to give the amide (IX). The piperidine ring nitrogen is protected against acylation by the acid (VII) during the reaction by means of a protecting group PG.

The amine-protecting group PG can be any protecting group known for use in protecting amine groups under the conditions used in the above process. Examples of protecting groups, and methods of protecting and deprotecting functional groups, can be found in Protective Groups in Organic Synthesis (T. Green and P. Wuts; 3rd Edition; John Wiley and Sons, 1999). Thus, for example, the piperidine ring nitrogen may be protected as an amide NCO—R) or a urethane (NCO—OR), for example, as: a methyl amide (NCO—CH₃); a benzyloxy amide (NCO—OCH₂C₆H₅, —NH-Cbz); as a tert-butoxy amide (—NCO—OC(CH₃)₃, N-Boc); a 2-biphenyl-2-propoxy amide (NCO—OC(CH₃)₂C₆H₄C₆H₅, N-Bpoc), as a 9-fluorenylmethoxy amide (N-Fmoc), as a 6-nitroveratryloxy amide (N-Nvoc), as a 2-trimethylsilylethyloxy amide (N-Teoc), as a 2,2,2-trichloroethyloxy amide (N-Troc), as an allyloxy amide (N-Alloc), or as a 2-(phenylsulphonyl)ethyloxy amide (—N-Psec). Other protecting groups for amines include toluenesulphonyl (tosyl) and methanesulphonyl (mesyl) groups and benzyl groups such as apara-methoxybenzyl (PMB) group. Preferred amine protecting groups are a urethane (NCO—OR), for example, a benzyloxy amide (NCO—OCH₂C₆H₅, —NH-Cbz), or a tert-butoxy amide (—NCO—OC(CH₃)₃, N-boc); an allyloxy amide (N-Alloc) or apara-methoxybenzyl (PMB) group. A particularly preferred protecting group PG is tert-butyloxycarbonyl (boc).

In the next step, the protecting group PG is removed from the amide (IX), in the case of a boc group using acidic conditions such as treatment with hydrogen chloride or hydrochloric acid in a polar solvent such as dioxane or ethyl acetate, to give the piperidine compound (X).

Following removal of the protecting group PG, the piperidine ring nitrogen atom is mesylated using methanesulphonyl chloride in the presence of a non-interfering base such as a tertiary amine (e.g. triethylamine) to give the nitro-compound (XI). The mesylation reaction is typically carried out in a polar aprotic solvent (such as acetonitrile or dioxane or dichloromethane or a mixture thereof) at a moderate temperature, for example room temperature or with mild heating, e.g. up to about 40-50° C.

The nitro group in the compound of the formula (XI) can then be reduced to an amino group by catalytic hydrogenation using hydrogen in the presence of a catalyst such as palladium on charcoal to give the amino compound (VI) which is then reacted with 2,6-dichlorobenzoic acid or 2,6-dichlorobenzoyl chloride under the conditions described above to give the compound of formula (I).

A further process for preparing a compound of the formula (I) comprises the reaction of a carboxylic acid of the formula (XII):

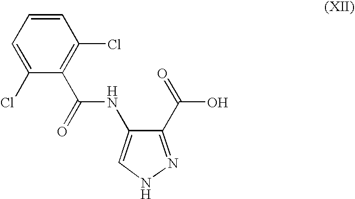

or an activated derivative thereof such as the acid chloride (i.e. compound (IV) above), with a compound of the formula (XIII):

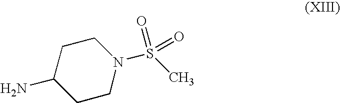

The reaction can be carried out under the amide coupling conditions described above, for example using EDC and HOBt as the amide coupling reagent in a polar solvent such as DMF in the presence of a non-interfering base such as triethylamine.

The compound (XIII) and its hydrochloride salt are commercially available, or compound (XIII) can be prepared by the sequence of reactions shown in Scheme 3 below.

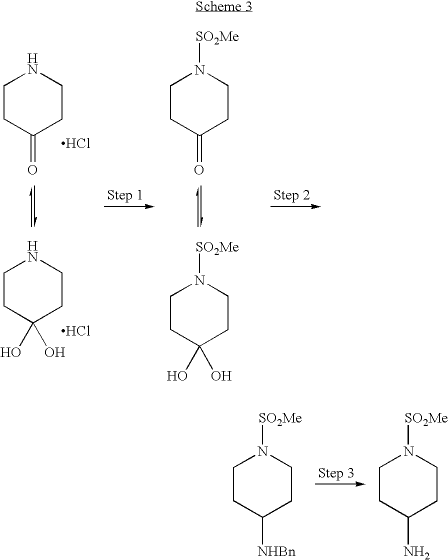

In Scheme 3, 4-piperidone monohydrate is reacted with methanesulphonyl chloride in the presence of a non-interfering base such as triethylamine in a polar solvent such as DMF, typically with heating to a non-extreme temperature, e.g. 40-50° C.

In step 2, the carbonyl group in the mesylpiperidone is subjected to a reductive amination using benzylamine in the presence of sodium triacetoxyborohydride. The benzyl group may then be removed by well known methods, e.g. hydrogenation in the presence of Pd/C catalyst, to give the desired compound (XIII).

Novel Pharmaceutical Formulations

The compound of the invention has good oral bioavailability but the oral bioavailability may be enhanced by the manner in which it is formulated.

The present invention provides improved pharmaceutical formulations that disintegrate rapidly to release the compound of the invention in a finely divided solid solution form in which it is readily absorbed.

Accordingly, in a further aspect, the invention provides a solid pharmaceutical composition comprising a compressed mixture of:

(a) a solid dispersion of 4-(2,6-dichloro-benzoylamino)-1H-pyrazole-3-carboxylic acid (1-methanesulphonyl-piperidin-4-yl)-amide in polyvinylpyrrolidone;

(b) a solid diluent: and

(c) a disintegrant; and optionally

(d) one or more further pharmaceutically acceptable excipients.

The solid pharmaceutical composition is typically presented in tablet or capsule form.

In one embodiment, the solid pharmaceutical composition is in the form of a tablet.

In another embodiment, the solid pharmaceutical composition is in the form of a tablet that can be either coated or uncoated

In another embodiment, the solid pharmaceutical composition is in the form of a capsule.

In another embodiment, the solid pharmaceutical composition is in the form of a capsule that can be a hard gelatin or HPMC capsule or a soft gelatin capsule, in particular it is a hard gelatin capsule.

The solid dispersion (a) contains 4-(2,6-dichloro-benzoylamino)-1H-pyrazole-3-carboxylic acid (1-methanesulphonyl-piperidin-4-yl)-amide dispersed in polyvinylpyrrolidone (PVP). The dispersion may take the form of a solid solution, or may consist of the compound of the invention dispersed as a finely divided solid in a surrounding matrix of PVP.

PVP is available in a range of molecular weights and a particular grade of PVP for use in the formulations of the present invention has a molecular weight in the range from 44,000-54,000.

The solid dispersion typically contains the compound of the invention and the PVP in a weight ratio of about 1:1 to about 1:6, more typically 1:2 to 1:4, for example a 1:3 ratio.

The solid dispersion can be prepared by dissolving the compound of the invention and the PVP in a common solvent (for example a solvent selected from chloroform, dichloromethane, methanol and ethanol and mixtures thereof (e.g. dichloromethane/ethanol in a 1:1 ratio) and then removing the solvent, for example on a rotary evaporator or by spray drying, in particular by spray drying the resulting solution.

The spray dried solid dispersion on its own typically has a very low density and the solid diluent assists in increasing the density of the composition, rendering it easier to compress. The solid diluent is typically a pharmacologically inert solid substance chosen from sugars or sugar alcohols, e.g. lactose, sucrose, sorbitol or mannitol; and non-sugar derived diluents such as sodium carbonate, calcium phosphate, calcium carbonate, and cellulose or derivatives thereof such as methyl cellulose, ethyl cellulose, hydroxypropyl methyl cellulose, and starches such as corn starch. An additional cellulose or cellulose derivative is micro-crystalline cellulose as discussed below.

Particular diluents are lactose and calcium phosphate. In particular the diluent is dibasic calcium phosphate.

The disintegrant is a substance that swells rapidly on contact with water so as to cause the rapid disintegration of the pharmaceutical composition and release of the compound of the invention.

Particular disintegrants are those known in the art as “super disintegrants” and include cross linked carboxymethylcellulose (Croscarmellose, also known as Croscarmellose sodium), cross-linked polyvinylpyrrolidone (cross-linked PVP or Crospovidone), and sodium starch glycolate. Examples of preferred super disintegrants are Croscarmellose and sodium starch glycolate.

Examples of other pharmaceutically acceptable excipients (d) that may be included in the pharmaceutical compositions of the invention include microcrystalline cellulose, which can act as both a diluent and an auxiliary disintegrant. Silicified microcrystalline cellulose (which contains about 1-3% silicon dioxide, typically about 2% silicon dioxide), may also be used to enhance the flowability of the composition and thereby improve the ease with which the composition can be compressed.

Another pharmaceutically acceptable excipient (d) that can be included in the compressed mixture is an alkali metal bicarbonate such as sodium bicarbonate. The bicarbonate reacts with acid in the stomach to release carbon dioxide thereby facilitating more rapid disintegration of the pharmaceutical composition.

Another example of other pharmaceutically acceptable excipients (d) that may be included in the pharmaceutical compositions of the invention include lubricants, such as magnesium stearate (e.g. 0.1-2%) or sodium stearyl fumarate (e.g. 0.1-5%), which may be added to aid the compression and encapsulation processes.

One particular mixture of components (a) to (d) is a mixture wherein:
component (a) is a spray dried solid dispersion of 4-(2,6-dichloro-benzoylamino)-1H-pyrazole-3-carboxylic acid (1-methanesulphonyl-piperidin-4-yl)-amide in PVP in a ratio of 1:3;
component (b) is calcium phosphate;
component (c) is Croscarmellose; and
component (d) is silicified microcrystalline cellulose.

In particular the mixture of components (a) to (d) is a mixture wherein:
component (a) is a spray dried solid dispersion of 4-(2,6-dichloro-benzoylamino)-1H-pyrazole-3-carboxylic acid (1-methanesulphonyl-piperidin-4-yl)-amide in PVP in a ratio of 1:3;
component (b) is dibasic calcium phosphate;
component (c) is Croscarmellose sodium; and
component (d) is silicified microcrystalline cellulose.

The mixture of components (a) to (c) and optionally (d) is compressed prior to processing to give the final dosage form. Thus, for example, it can be compressed to give a compressed solid mass (e.g. in the form of a ribbon or pellet) and then milled to form granules of a desired particle size. The granules can then be filled into a capsule or shaped and compressed to form a tablet.

The mixture of components (a) to (c) and optionally (d) can be compressed by means of various methods well known to the skilled person. For example, they can be compressed using a roller compactor to form a ribbon which can then be broken up and milled to form granules. Alternatively they can be compressed using a tablet compression machine into slugs that can be broken up and milled to form granules.

In one embodiment, the invention provides a pharmaceutical composition in the form of a capsule containing a milled compressed mixture of components (a) to (c) and optionally (d) as defined herein.

In another embodiment, the invention provides a pharmaceutical composition in the form of a tablet comprising a compressed mixture of components (a) to (c) and optionally (d) as defined herein.

One aspect of the invention is a solid pharmaceutical composition comprising a compressed mixture of:

(a) a solid dispersion of 4-(2,6-dichloro-benzoylamino)-1H-pyrazole-3-carboxylic acid (1-methanesulphonyl-piperidin-4-yl)-amide in polyvinylpyrrolidone;

(b) a solid diluent: and

(c) a disintegrant; and optionally

(d) one or more further pharmaceutically acceptable excipients.

The solid dispersion (a) in the pharmaceutical composition typically constitutes 10-70% w/w of the total weight of the composition. For example, the solid dispersion may constitute 20-60% w/w, or 25-55%, or 30-50% or 25-40% w/w of the composition.

The amount of excipient (b) contained in the composition may be in the range 5-95% in particular 10-70% w/w, particularly 20-60% or 30-40% e.g. 33-36%. The ratio of Compound/PVP to excipient (b) is typically in the range 5:1 to 1:5, in particular in the weight ratio 2:1 or 1:1.

The amount of excipient (c) contained in the composition may be in the range 1-30% w/w, in particular 5-25% e.g. 10-25% such as 12-20%. The ratio of Compound/PVP to excipient (c) is typically in the range 5:1 to 1:5, in particular in the weight ratio 3:1 or 2:1.

The amount of excipient (d), when present, contained in the composition may be in the range 0.1-20%, in particular 1-20% w/w, particularly 5-15% e.g. 11 or 12%. The ratio of Compound/PVP to (d) is typically in the range 5:1 to 1:5, in particular in the weight ratio 3:1 or 2:1.

Accordingly, in a further aspect, the invention provides a solid pharmaceutical composition comprising a compressed mixture of:

(a) 10-70% w/w of solid dispersion of 4-(2,6-dichloro-benzoylamino)-1H-pyrazole-3-carboxylic acid (1-methanesulphonyl-piperidin-4-yl)-amide in polyvinylpyrrolidone;

(b) 10-70% w/w of a solid diluent: and

(c) 1-20% w/w of a disintegrant; and optionally

(d) 1-30% w/w of one or more further pharmaceutically acceptable excipients.

It will be appreciated that for each composition, the sum of the weight percentages of the individual components (a), (b), (c) and (d) will give a total of 100%.

In one embodiment, the diluent (b) (e.g. dicalcium phosphate) comprises 30-40% by weight of the total weight of the pharmaceutical composition.

In one embodiment the pharmaceutical composition comprises 10-30% disintegrant (c) in particular where the disintegrant is Croscarmellose sodium. In another embodiment the pharmaceutical composition comprises 10-20% e.g. 12% Croscarmellose sodium blended in the composition and a further 5-20% wt e.g. 10% wt Croscarmellose sodium mixed with the blended composition.

In one embodiment the pharmaceutical composition comprises 10-20% of one or more further pharmaceutically acceptable excipients. In one embodiment the further pharmaceutically acceptable excipient is 10-20% silicified microcrystalline cellulose.

In one embodiment the ratio of (a) and excipient (b) is approximately 1:1. In another embodiment the ratio of excipients (c) and (d), when present, is approximately 1:1. In one particular embodiment the ratio of all the components ((a):(b):(c):(d)) in the composition is approximately 3-4:3-4:1-2:1-2 e.g. 3.9:3.6:1.2:1.2.

Biological Activity

The compound of the formula (I), i.e. 4-(2,6-dichloro-benzoylamino)-1H-pyrazole-3-carboxylic acid (1-methanesulphonyl-piperidin-4-yl)-amide, is an inhibitor of cyclin dependent kinases. For example, the compound of formula (I) is an inhibitor of cyclin dependent kinases selected from CDK1, CDK2, CDK3, CDK4, CDK5, CDK6 and CDK9, and more particularly selected from CDK1, CDK2, CDK3, CDK4, CDK5 and CDK9.

The compound of the formula (I) also has activity against glycogen synthase kinase-3 (GSK-3).

As a consequence of their activity in modulating or inhibiting CDK and glycogen synthase kinase, the compound of formula (I) will be useful in providing a means of arresting, or recovering control of, the cell cycle in abnormally dividing cells. The compound will therefore prove useful in treating or preventing proliferative disorders such as cancers. The compound of the invention will also be useful in treating conditions such as viral infections, type II or non-insulin dependent diabetes mellitus, autoimmune diseases, head trauma, stroke, epilepsy, neurodegenerative diseases such as Alzheimer's, motor neurone disease, progressive supranuclear palsy, corticobasal degeneration and Pick's disease, for example autoimmune diseases and neurodegenerative diseases.

One sub-group of disease states and conditions where the compounds of the invention will be useful consists of viral infections, autoimmune diseases and neurodegenerative diseases.

CDKs play a role in the regulation of the cell cycle, apoptosis, transcription, differentiation and CNS function. Therefore, CDK inhibitors could be useful in the treatment of diseases in which there is a disorder of proliferation, apoptosis or differentiation such as cancer. In particular RB+ve tumours may be particularly sensitive to CDK inhibitors. RB−ve tumours may also be sensitive to CDK inhibitors.

Examples of cancers which may be inhibited include, but are not limited to, a carcinoma, for example a carcinoma of the bladder, breast, colon (e.g. colorectal carcinomas such as colon adenocarcinoma and colon adenoma), kidney, epidermis, liver, lung, for example adenocarcinoma, small cell lung cancer and non-small cell lung carcinomas, oesophagus, gall bladder, ovary, pancreas e.g. exocrine pancreatic carcinoma, stomach, cervix, thyroid, prostate, or skin, for example squamous cell carcinoma; a hematopoietic tumour of lymphoid lineage, for example leukemia, acute lymphocytic leukemia, chronic lymphocytic leukaemia, B-cell lymphoma (such as diffuse large B cell lymphoma), T-cell lymphoma, Hodgkin's lymphoma, non-Hodgkin's lymphoma, hairy cell lymphoma, or Burkett's lymphoma; a hematopoietic tumour of myeloid lineage, for example acute and chronic myelogenous leukemias, myelodysplastic syndrome, or promyelocytic leukemia; thyroid follicular cancer; a tumour of mesenchymal origin, for example fibrosarcoma or habdomyosarcoma; a tumour of the central or peripheral nervous system, for example astrocytoma, neuroblastoma, glioma or schwannoma; melanoma; seminoma; teratocarcinoma; osteosarcoma; xeroderma pigmentosum; keratoctanthoma; thyroid follicular cancer; or Kaposi's sarcoma.

The cancers may be cancers which are sensitive to inhibition of any one or more cyclin dependent kinases selected from CDK1, CDK2, CDK3, CDK4, CDK5 and CDK6, for example, one or more CDK kinases selected from CDK1, CDK2, CDK4 and CDK5, e.g. CDK1 and/or CDK2.

Whether or not a particular cancer is one which is sensitive to inhibition by a cyclin dependent kinase may be determined by means of a cell growth assay as set out in the examples below or by a method as set out in the section headed “Methods of Diagnosis”.

CDKs are also known to play a role in apoptosis, proliferation, differentiation and transcription and therefore CDK inhibitors could also be useful in the treatment of the following diseases other than cancer; viral infections, for example herpes virus, pox virus, Epstein-Barr virus, Sindbis virus, adenovirus, HIV, HPV, HCV and HCMV; prevention of AIDS development in HIV-infected individuals; chronic inflammatory diseases, for example systemic lupus erythematosus, autoimmune mediated glomerulonephritis, rheumatoid arthritis, psoriasis, inflammatory bowel disease, and autoimmune diabetes mellitus; cardiovascular diseases for example cardiac hypertrophy, restenosis, atherosclerosis; neurodegenerative disorders, for example Alzheimer's disease, AIDS-related dementia, Parkinson's disease, amyotropic lateral sclerosis, retinitis pigmentosa, spinal muscular atropy and cerebellar degeneration; glomerulonephritis; myelodysplastic syndromes, ischemic injury associated myocardial infarctions, stroke and reperfusion injury, arrhythmia, atherosclerosis, toxin-induced or alcohol related liver diseases, haematological diseases, for example, chronic anemia and aplastic anemia; degenerative diseases of the musculoskeletal system, for example, osteoporosis and arthritis, aspirin-sensitive rhinosinusitis, cystic fibrosis, multiple sclerosis, kidney diseases and cancer pain.

It has also been discovered that some cyclin-dependent kinase inhibitors can be used in combination with other anticancer agents. For example, the cyclin-dependent kinase inhibitor flavopiridol has been used with other anticancer agents in combination therapy.

Thus, in the pharmaceutical compositions, uses or methods of this invention for treating a disease or condition comprising abnormal cell growth, the disease or condition comprising abnormal cell growth in one embodiment is a cancer.

One group of cancers includes human breast cancers (e.g. primary breast tumours, node-negative breast cancer, invasive duct adenocarcinomas of the breast, non-endometrioid breast cancers); and mantle cell lymphomas. In addition, other cancers are colorectal and endometrial cancers.

Another sub-set of cancers includes hematopoietic tumours of lymphoid lineage, for example leukemia, chronic lymphocytic leukaemia, mantle cell lymphoma and B-cell lymphoma (such as diffuse large B cell lymphoma).

One particular cancer is chronic lyniphocytic leukaemia.

Another particular cancer is mantle cell lymphoma.

Another particular cancer is diffuse large B cell lymphoma

Another sub-set of cancers includes breast cancer, ovarian cancer, colon cancer, prostate cancer, oesophageal cancer, squamous cancer and non-small cell lung carcinomas.

The activity of the compound of the invention as an inhibitor of cyclin dependent kinases and glycogen synthase kinase-3 can be measured using the assays set forth in the examples below and the level of activity exhibited by a given compound can be defined in terms of the IC₅₀ value.

### ADVANTAGES OF THE COMPOUNDS OF THE INVENTION

The compound 4-(2,6-dichloro-benzoylamino)-1H-pyrazole-3-carboxylic acid (1-methanesulphonyl-piperidin-4-yl)-amide, has advantages over prior art compounds.

The compound of the invention has physicochemical properties suitable for oral exposure.

The compound of the invention has a higher IC₅₀ for transcription than IC₅₀ for proliferation in HCT-116 cells: thus, for example, the IC₅₀ for transcription is ˜100-fold higher than the IC₅₀ for proliferation. This is advantageous as the compound could be better tolerated thus allowing it to be dosed at higher levels and for longer doses.

In particular, the compound of the formula (I) exhibits improved oral bioavailability relative to prior art compounds. Oral bioavailability can be defined as the ratio (F) of the plasma exposure of a compound when dosed by the oral route to the plasma exposure of the compound when dosed by the intravenous (i.v.) route, expressed as a percentage.

Compounds having an oral bioavailability (F value) of greater than 30%, more preferably greater than 40%, are particularly advantageous in that they may be adminstered orally rather than, or as well as, by parenteral administration. The compound 4-(2,6-dichloro-benzoylamino)-1H-pyrazole-3-carboxylic acid (1-methanesulphonyl-piperidin-4-yl)-amide has 30-100% bioavailability, in particular 40-50% bioavailability, when administered to mice by the oral route.

The compound 4-(2,6-dichloro-benzoylamino)-1H-pyrazole-3-carboxylic acid (1-methanesulphonyl-piperidin-4-yl)-amide, has greater in vitro kinase (CDK2) inhibitory activity and more potent anti-proliferative effects on cancer cell lines. In addition, the compound has lower activity versus GSK3β and is more selective for CDK2 over GSK3β. Therefore the action of the compound is dominated by cell cycle effects via the CDK inhibition and not complicated by the additional consequences of GSK3beta inhibition on, for example, insulin sensitivity, growth factor action. The compound therefore has a cleaner cell cycle inhibition profile and fewer side effects from the additional effects via GSK3 beta. A comparison of the biological properties of the compound 4-(2,6-dichloro-benzoylamino)-1H-pyrazole-3-carboxylic acid (1-methanesulphonyl-piperidin-4-yl)-amide with the properties of its 2,6-difluorobenzoylamino analogue is set out in Example 12 below.

The activity of the compound of the invention as an inhibitor of cyclin dependent kinases and glycogen synthase kinase-3 can be measured using the assays set forth in the examples below and the level of activity exhibited can be defined in terms of the IC₅₀ value.

Thus, for example, the compound of the invention will be useful in alleviating or reducing the incidence of cancer.

Accordingly, the invention also provides inter alia:
4-(2,6-Dichloro-benzoylamino)-1H-pyrazole-3-carboxylic acid (1-methanesulphonyl-piperidin-4-yl)-amide in a substantially crystalline form as defined herein, for use in the prophylaxis or treatment of a disease state or condition mediated by a cyclin dependent kinase or glycogen synthase kinase-3 (preferably a cyclin dependent kinase).
4-(2,6-Dichloro-benzoylamino)-1H-pyrazole-3-carboxylic acid (1-methanesulphonyl-piperidin-4-yl)-amide in a substantially crystalline form as defined herein, for use in inhibiting tumour growth in a mammal.
4-(2,6-Dichloro-benzoylamino)-1H-pyrazole-3-carboxylic acid (1-methanesulphonyl-piperidin-4-yl)-amide in a substantially crystalline form as defined herein, for use in inhibiting the growth of tumour cells (e.g. in a mammal).
A method for the prophylaxis or treatment of a disease state or condition mediated by a cyclin dependent kinase or glycogen synthase kinase-3 (preferably a cyclin dependent kinase), which method comprises administering to a subject in need thereof4-(2,6-dichloro-benzoylamino)-1H-pyrazole-3-carboxylic acid (1-methanesulphonyl-piperidin-4-yl)-amide in a substantially crystalline form as defined herein.
A method of inhibiting tumour growth in a mammal (e.g. a human), which method comprises administering to the mammal (e.g. a human) an effective tumour growth-inhibiting amount of 4-(2,6-dichloro-benzoylamino)-1H-pyrazole-3-carboxylic acid (1-methanesulphonyl-piperidin-4-yl)-amide in a substantially crystalline form as defined herein.
A method of inhibiting the growth of tumour cells (e.g. tumour cells present in a mammal such as a human), which method comprises contacting the tumour cells with an effective tumour cell growth-inhibiting amount of 4-(2,6-dichloro-benzoylamino)-1H-pyrazole-3-carboxylic acid (1-methanesulphonyl-piperidin-4-yl)-amide in a substantially crystalline form as defined herein.
A method for alleviating or reducing the incidence of a disease state or condition mediated by a cyclin dependent kinase or glycogen synthase kinase-3 (preferably a cyclin dependent kinase), which method comprises administering to a subject in need thereof 4-(2,6-dichloro-benzoylamino)-1H-pyrazole-3-carboxylic acid (1-methanesulphonyl-piperidin-4-yl)-amide in a substantially crystalline form as defined herein.
A method for treating a disease or condition comprising or arising from abnormal cell growth in a mammal, which method comprises administering to the mammal 4-(2,6-dichloro-benzoylamino)-1H-pyrazole-3-carboxylic acid (1-methanesulphonyl-piperidin-4-yl)-amide in a substantially crystalline form as defined herein, in an amount effective in inhibiting abnormal cell growth.
A method for alleviating or reducing the incidence of a disease or condition comprising or arising from abnormal cell growth in a mammal, which method comprises administering to the mammal 4-(2,6-dichloro-benzoylamino)-1H-pyrazole-3-carboxylic acid (1-methanesulphonyl-piperidin-4-yl)-amide in a substantially crystalline form as defined herein, in an amount effective in inhibiting abnormal cell growth.
A method for treating a disease or condition comprising or arising from abnormal cell growth in a mammal, the method comprising administering to the mammal 4-(2,6-dichloro-benzoylamino)-1H-pyrazole-3-carboxylic acid (1-methanesulphonyl-piperidin-4-yl)-amide in a substantially crystalline form as defined herein, in an amount effective to inhibit a cdk kinase (such as cdk1 or cdk2) or glycogen synthase kinase-3 activity.
A method for alleviating or reducing the incidence of a disease or condition comprising or arising from abnormal cell growth in a mammal, the method comprising administering to the mammal 4-(2,6-dichloro-benzoylamino)-1H-pyrazole-3-carboxylic acid (1-methanesulphonyl-piperidin-4-yl)-amide in a substantially crystalline form as defined herein, in an amount effective to inhibit a cdk kinase (such as cdk1 or cdk2) or glycogen synthase kinase-3 activity.
A method of inhibiting a cyclin dependent kinase or glycogen synthase kinase-3, which method comprises contacting the kinase with 4-(2,6-dichloro-benzoylamino)-1H-pyrazole-3-carboxylic acid (1-methanesulphonyl-piperidin-4-yl)-amide in a substantially crystalline form as defined herein.
A method of modulating a cellular process (for example cell division) by inhibiting the activity of a cyclin dependent kinase or glycogen synthase kinase-3 (preferably a cyclin dependent kinase) using 4-(2,6-dichloro-benzoylamino)-1H-pyrazole-3-carboxylic acid (1-methanesulphonyl-piperidin-4-yl)-amide in a substantially crystalline form as defined herein.
4-(2,6-Dichloro-benzoylamino)-1H-pyrazole-3-carboxylic acid (1-methanesulphonyl-piperidin-4-yl)-amide in a substantially crystalline form as defined herein for use in the prophylaxis or treatment of a disease state as described herein.
The use of 4-(2,6-dichloro-benzoylamino)-1H-pyrazole-3-carboxylic acid (1-methanesulphonyl-piperidin-4-yl)-amide in a substantially crystalline form as defined herein, for the manufacture of a medicament, wherein the medicament is for any one or more of the uses defined herein.
A pharmaceutical composition comprising 4-(2,6-dichloro-benzoylamino)-1H-pyrazole-3-carboxylic acid (1-methanesulphonyl-piperidin-4-yl)-amide in a substantially crystalline form as defined herein and a pharmaceutically acceptable carrier.
4-(2,6-Dichloro-benzoylamino)-1H-pyrazole-3-carboxylic acid (1-methanesulphonyl-piperidin-4-yl)-amide in a substantially crystalline form as defined herein, for use in medicine.
4-(2,6-Dichloro-benzoylamino)-1H-pyrazole-3-carboxylic acid (1-methanesulphonyl-piperidin-4-yl)-amide in a substantially crystalline form as defined herein, for any of the uses and methods set forth above, and as described elsewhere herein.
A method for the diagnosis and treatment of a disease state or condition mediated by a cyclin dependent kinase, which method comprises (i) screening a patient to determine whether a disease or condition from which the patient is or may be suffering is one which would be susceptible to treatment with a compound having activity against cyclin dependent kinases; and (ii) where it is indicated that the disease or condition from which the patient is thus susceptible, thereafter administering to the patient 4-(2,6-dichloro-benzoylamino)-1H-pyrazole-3-carboxylic acid (1-methanesulphonyl-piperidin-4-yl)-amide in a substantially crystalline form as defined herein.
The use of 4-(2,6-dichloro-benzoylamino)-1H-pyrazole-3-carboxylic acid (1-methanesulphonyl-piperidin-4-yl)-amide in a substantially crystalline form as defined herein for the manufacture of a medicament for the treatment or prophylaxis of a disease state or condition in a patient who has been screened and has been determined as suffering from, or being at risk of suffering from, a disease or condition which would be susceptible to treatment with a compound having activity against cyclin dependent kinase.

In this application, unless the context indicates otherwise, references to a compound of formula (I) includes all subgroups of formula (I) as defined herein and the term ‘subgroups’ includes all preferences, embodiments, examples and particular compounds defined herein. Any references to formula (I) herein shall also be taken to refer to and any sub-group of compounds within formula (I) and any preferences and examples thereof unless the context requires otherwise.

As used herein, the term “modulation”, as applied to the activity of cyclin dependent kinase (CDK) and glycogen synthase kinase (GSK, e.g. GSK-3), is intended to define a change in the level of biological activity of the kinase(s). Thus, modulation encompasses physiological changes which effect an increase or decrease in the relevant kinase activity. In the latter case, the modulation may be described as “inhibition”. The modulation may arise directly or indirectly, and may be mediated by any mechanism and at any physiological level, including for example at the level of gene expression (including for example transcription, translation and/or post-translational modification), at the level of expression of genes encoding regulatory elements which act directly or indirectly on the levels of cyclin dependent kinase (CDK) and/or glycogen synthase kinase-3 (GSK-3) activity, or at the level of enzyme (e.g. cyclin dependent kinase (CDK) and/or glycogen synthase kinase-3 (GSK-3)) activity (for example by allosteric mechanisms, competitive inhibition, active-site inactivation, perturbation of feedback inhibitory pathways etc.). Thus, modulation may imply elevated/suppressed expression or over- or under-expression of the cyclin dependent kinase (CDK) and/or glycogen synthase kinase-3 (GSK-3), including gene amplification (i.e. multiple gene copies) and/or increased or decreased expression by a transcriptional effect, as well as hyper- (or hypo-)activity and (de)activation of the cyclin dependent kinase (CDK) and/or glycogen synthase kinase-3 (GSK-3) (including (de)activation) by mutation(s). The terms “modulated”, “modulating” and “modulate” are to be interpreted accordingly.

As used herein, the term “mediated”, as used e.g. in conjunction with the cyclin dependent kinases (CDK) and/or glycogen synthase kinase-3 (GSK-3) as described herein (and applied for example to various physiological processes, diseases, states, conditions, therapies, treatments or interventions) is intended to operate limitatively so that the various processes, diseases, states, conditions, treatments and interventions to which the term is applied are those in which cyclin dependent kinase (CDK) and/or glycogen synthase kinase-3 (GSK-3) plays a biological role. In cases where the term is applied to a disease, state or condition, the biological role played by cyclin dependent kinase (CDK) and/or glycogen synthase kinase-3 (GSK-3) may be direct or indirect and may be necessary and/or sufficient for the manifestation of the symptoms of the disease, state or condition (or its aetiology or progression). Thus, cyclin dependent kinase (CDK) and/or glycogen synthase kinase-3 (GSK-3) activity (and in particular aberrant levels of cyclin dependent kinase (CDK) and/or glycogen synthase kinase-3 (GSK-3)activity, e.g. cyclin dependent kinases (CDK) and/or glycogen synthase kinase-3 (GSK-3) over-expression) need not necessarily be the proximal cause of the disease, state or condition: rather, it is contemplated that the CDK- and/or GSK- (e.g. GSK-3-) mediated diseases, states or conditions include those having multifactorial aetiologies and complex progressions in which CDK and/or GSK-3 is only partially involved. In cases where the term is applied to treatment, prophylaxis or intervention (e.g. in the “CDK-mediated treatments” and “GSK-3-mediated prophylaxis” of the invention), the role played by CDK and/or GSK-3 may be direct or indirect and may be necessary and/or sufficient for the operation of the treatment, prophylaxis or outcome of the intervention. Thus, a disease state or condition mediated by the cyclin dependent kinases (CDK) and/or glycogen synthase kinase-3 (GSK-3) as described herein includes a disease state or condition which has arisen as a consequence of the development of resistance to any particular cancer drug or treatment (including in particular resistance to one or more of the ancillary compounds described herein).

Pharmaceutical Formulations

While it is possible for the substantially crystalline 4-(2,6-dichloro-benzoylamino)-1H-pyrazole-3-carboxylic acid (1-methanesulphonyl-piperidin-4-yl)-amide as defined herein or 4-(2,6-dichloro-benzoylamino)-1H-pyrazole-3-carboxylic acid (1-methanesulphonyl-piperidin-4-yl)-amide prepared by the novel processes of the invention to be administered alone, it is preferable to present the compound in the form of a pharmaceutical composition (e.g. formulation).

Particular examples of pharmaceutical compositions are described in the section above headed “Novel Pharmaceutical Formulations”. However, on a more general basis, the compound 4-(2,6-dichloro-benzoylamino)-1H-pyrazole-3-carboxylic acid (1-methanesulphonyl-piperidin-4-yl)-amide can be formulated in a pharmaceutical composition together with one or more pharmaceutically acceptable carriers, adjuvants, excipients, diluents, fillers, buffers, stabilisers, preservatives, lubricants, or other materials well known to those skilled in the art. The compositions may also include other therapeutic or prophylactic agents, for example agents that reduce or alleviate some of the side effects associated with chemotherapy. Particular examples of such agents include anti-emetic agents and agents that prevent or decrease the duration of chemotherapy-associated neutropenia and prevent complications that arise from reduced levels of red blood cells or white blood cells, for example erythropoietin (EPO), granulocyte macrophage-colony stimulating factor (GM-CSF), and granulocyte-colony stimulating factor (G-CSF).

Thus, the present invention further provides pharmaceutical compositions, as defined above, and methods of making a pharmaceutical composition comprising admixing a compound of the invention, e.g. the compound 4-(2,6-dichloro-benzoylamino)-1H-pyrazole-3-carboxylic acid (1-methanesulphonyl-piperidin-4-yl)-amide in substantially crystalline form, together with one or more pharmaceutically acceptable carriers, excipients, buffers, adjuvants, stabilizers, or other materials, as described herein.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Accordingly, in a further aspect, the invention provides the compound 4-(2,6-dichloro-benzoylamino)-1H-pyrazole-3-carboxylic acid (1-methanesulphonyl-piperidin-4-yl)-amide in a substantially crystalline form in the form of a pharmaceutical composition, i.e. a solid or semi-solid formulation.

The pharmaceutical compositions can be in any form suitable for oral, parenteral, topical, intranasal, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery. The delivery can be by bolus injection, short term infusion or longer term infusion and can be via passive delivery or through the utilisation of a suitable infusion pump.

Pharmaceutical formulations adapted for parenteral administration include aqueous and non-aqueous sterile injection solutions which may contain anti-oxidants, buffers, bacteriostats, co-solvents, surface active agents, organic solvent mixtures, cyclodextrin complexation agents, emulsifying agents (for forming and stabilizing emulsion formulations), liposome components for forming liposomes, gellable polymers for forming polymeric gels, lyophilisation protectants and combinations of agents for, inter alia, stabilising the active ingredient in a soluble form and rendering the formulation isotonic with the blood of the intended recipient. Pharmaceutical formulations for parenteral administration may also take the form of aqueous and non-aqueous sterile suspensions which may include suspending agents and thickening agents (R. G. Strickly, Solubilizing Excipients in oral and injectable formulations, Pharmaceutical Research, Vol 21(2) 2004, p 201-230).

A drug molecule that is ionizable can be solubilized to the desired concentration by pH adjustment if the drug's pKa is sufficiently away from the formulation pH value. The acceptable range is pH 2-12 for intravenous and intramuscular administration, but subcutaneously the range is pH 2.7-9.0. The solution pH is controlled by either the salt form of the drug, strong acids/bases such as hydrochloric acid or sodium hydroxide, or by solutions of buffers which include but are not limited to buffering solutions formed from glycine, citrate, acetate, maleate, succinate, histidine, phosphate, tris(hydroxymethyl)aminomethane (TRIS), or carbonate.

The combination of an aqueous solution and a water-soluble organic solvent/surfactant (i.e., a cosolvent) is often used in injectable formulations. The water-soluble organic solvents and surfactants used in injectable formulations include but are not limited to propylene glycol, ethanol, polyethylene glycol 300, polyethylene glycol 400, glycerin, dimethylacetamide (DMA), N-methyl-2-pyrrolidone (NMP; Pharmasolve), dimethylsulphoxide (DMSO), Solutol HS 15, Cremophor EL, Cremophor RH 60, and polysorbate 80. Such formulations can usually be, but are not always, diluted prior to injection.

Propylene glycol, PEG 300, ethanol, Cremophor EL, Cremophor RH 60, and polysorbate 80 are the entirely organic water-miscible solvents and surfactants used in commercially available injectable formulations and can be used in combinations with each other. The resulting organic formulations are usually diluted at least 2-fold prior to IV bolus or IV infusion.

Alternatively increased water solubility can be achieved through molecular complexation with cyclodextrins.

Liposomes are closed spherical vesicles composed of outer lipid bilayer membranes and an inner aqueous core and with an overall diameter of <100 μm. Depending on the level of hydrophobicity, moderately hydrophobic drugs can be solubilized by liposomes if the drug becomes encapsulated or intercalated within the liposome. Hydrophobic drugs can also be solubilized by liposomes if the drug molecule becomes an integral part of the lipid bilayer membrane, and in this case, the hydrophobic drug is dissolved in the lipid portion of the lipid bilayer. A typical liposome formulation contains water with phospholipid at −5-20 mg/ml, an isotonicifier, a pH 5-8 buffer, and optionally cholesterol.

The formulations may be presented in unit-dose or multi-dose containers, for example sealed ampoules, vials and prefilled syringes, and may be stored in a freeze-dried (lyophilised) condition requiring only the addition of the sterile liquid carrier, for example water for injections, immediately prior to use.

The pharmaceutical formulation can be prepared by lyophilising a compound of the invention. Lyophilisation refers to the procedure of freeze-drying a composition. Freeze-drying and lyophilisation are therefore used herein as synonyms. A typical process is to solubilise the compound and the resulting formulation is clarified, sterile filtered and aseptically transferred to containers appropriate for lyophilisation (e.g. vials). In the case of vials, they are partially stoppered with lyo-stoppers. The formulation can be cooled to freezing and subjected to lyophilisation under standard conditions and then hermetically capped forming a stable, dry lyophile formulation. The composition will typically have a low residual water content, e.g. less than 5% e.g. less than 1% by weight based on weight of the lyophile.

The lyophilisation formulation may contain other excipients for example, thickening agents, dispersing agents, buffers, antioxidants, preservatives, and tonicity adjusters. Typical buffers include phosphate, acetate, citrate and glycine. Examples of antioxidants include ascorbic acid, sodium bisulphite, sodium metabisulphite, monothioglycerol, thiourea, butylated hydroxytoluene, butylated hydroxyl anisole, and ethylenediamietetraacetic acid salts. Preservatives may include benzoic acid and its salts, sorbic acid and its salts, alkyl esters of para-hydroxybenzoic acid, phenol, chlorobutanol, benzyl alcohol, thimerosal, benzalkonium chloride and cetylpyridinium chloride. The buffers mentioned previously, as well as dextrose and sodium chloride, can be used for tonicity adjustment if necessary.

Bulking agents are generally used in lyophilisation technology for facilitating the process and/or providing bulk and/or mechanical integrity to the lyophilized cake. Bulking agent means a freely water soluble, solid particulate diluent that when co-lyophilised with the compound or salt thereof, provides a physically stable lyophilized cake, a more optimal freeze-drying process and rapid and complete reconstitution. The bulking agent may also be utilised to make the solution isotonic.

The water-soluble bulking agent can be any of the pharmaceutically acceptable inert solid materials typically used for lyophilisation. Such bulking agents include, for example, sugars such as glucose, maltose, sucrose, trehalose and lactose; polyalcohols such as sorbitol or mannitol; amino acids such as glycine; polymers such as polyvinylpyrrolidine; and polysaccharides such as dextran.

The ratio of the weight of the bulking agent to the weight of active compound is typically within the range from about 1 to about 5, for example of about 1 to about 3, e.g. in the range of about 1 to 2.

Alternatively it can be provided in a solution form which may be concentrated and sealed in a suitable vial. Sterilisation of dosage forms may be via filtration or by autoclaving of the vials and their contents at appropriate stages of the formulation process. The supplied formulation may require further dilution or preparation before delivery for example dilution into suitable sterile infusion packs.

Extemporaneous injection solutions and suspensions may be prepared from sterile powders, granules and tablets.

Pharmaceutical dosage forms suitable for oral administration are preferred and such formulations include tablets (such as coated or uncoated), capsules (such as hard or soft shell), caplets, pills, lozenges, syrups, solutions, powders, granules, elixirs and suspensions, sublingual tablets, wafers or patches such as buccal patches.

Pharmaceutical compositions containing compounds of the invention can be formulated in accordance with known techniques, see for example, Remington's Pharmaceutical Sciences, Mack Publishing Company, Easton, Pa., USA.

Thus, tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a cellulose or derivative thereof such as microcrystalline cellulose (MCC), methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (eg; tablets, capsules etc.) can be coated or un-coated, but typically have a coating, for example a protective film coating (e.g. a polymer, wax or varnish) or a release controlling coating. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent which may be adapted to release the compound in a controlled manner in the gastrointestinal tract or the drug can be presented in a polymer coating e.g. a polymethacrylate polymer coating, comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. As a further alternative, the active compound can be formulated in a delivery system that provides osmotic control of the release of the compound. Osmotic release and other delayed release or sustained release formulations may be prepared in accordance with methods well known to those skilled in the art.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragees, tablets or capsules.

Pharmaceutical compositions for oral administration can be obtained by combining the active ingredient with solid carriers, if desired granulating a resulting mixture, and processing the mixture, if desired or necessary, after the addition of appropriate excipients, into tablets, dragee cores or capsules. It is also possible for them to be incorporated into plastics carriers that allow the active ingredients to diffuse or be released in measured amounts.

The compound of the invention can also be formulated as a solid dispersion. Solid dispersions are homogeneous extremely fine disperse phases of two or more solids. Solid solutions (molecularly disperse systems), one type of solid dispersion, are well known for use in pharmaceutical technology (see Chiou and Riegelman, J. Pharm. Sci., 60, 1281-1300 (1971)) and are useful in increasing dissolution rates and increasing the bioavailability of poorly water-soluble drugs.

Solid dispersions of drugs are generally produced by melt or solvent evaporation methods. For melt processing, the materials (excipients) which are usually semisolid and waxy in nature, are heated to cause melting and dissolution of the drug substance, followed by hardening by cooling to very low temperatures. The solid dispersion can then be pulverized, sieved, mixed with excipients, and encapsulated into hard gelatin capsules or compressed into tablets. Alternatively the use of surface-active and self-emulsifying carriers allows the encapsulation of solid dispersions directly into hard gelatin capsules as melts. Alternatively the use of waxes, or low melting point polymers allows the encapsulation of solid dispersions directly into hard or soft gelatin capsules as melts. Solid plugs are formed inside the capsules when the melts are cooled to room temperature.

Solid solutions can also be manufactured by dissolving the drug and the required excipient in either an aqueous solution or a pharmaceutically acceptable organic solvent, followed by removal of the solvent, using a pharmaceutically acceptable method, such as spray drying. The resulting solid can be particle sized if required, optionally mixed with exipients and either made into tablets or filled into capsules.

A particularly suitable polymeric auxiliary for producing such solid dispersions or solid solutions is polyvinylpyrrolidone (PVP).

The pharmaceutical composition can comprise a substantially amorphous solid solution, said solid solution comprising

(a) a compound of the formula (I), for example the compound of Example 1; and

(b) a polymer selected from the group consisting of:

polyvinylpyrrolidone (povidone), crosslinked polyvinylpyrrolidone (crospovidone), hydroxypropyl methylcellulose, hydroxypropylcellulose, polyethylene oxide, gelatin, crosslinked polyacrylic acid (carbomer), carboxymethylcellulose, crosslinked carboxymethylcellulose (croscarmellose), methylcellulose, methacrylic acid copolymer, methacrylate copolymer, and water soluble salts such as sodium and ammonium salts of methacrylic acid and methacrylate copolymers, cellulose acetate phthalate, hydroxypropylmethylcellulose phthalate and propylene glycol alginate;

wherein the ratio of said compound to said polymer is about 1:1 to about 1:6, for example a 1:3 ratio, spray dried from a mixture of one of chloroform or dichloromethane and one of methanol or ethanol, preferably dichloromethane/ethanol in a 1:1 ratio.

In another embodiment the pharmaceutical composition can comprise a substantially amorphous solid solution, said solid solution comprising

(a) a compound of the formula (I), for example the compound of Example 1; and

(b) a polymer selected from the group consisting of:

polyvinylpyrrolidone (povidone), hydroxypropyl methylcellulose, hydroxypropylcellulose, polyethylene glycol, polyethylene oxide, gelatin, crosslinked polyacrylic acid (carbomer), carboxymethylcellulose, methylcellulose, methacrylic acid copolymer, methacrylate copolymer, and water soluble salts such as sodium and ammonium salts of methacrylic acid and methacrylate copolymers, cellulose acetate phthalate, hydroxypropylmethylcellulose phthalate and propylene glycol alginate;

wherein the ratio of said compound to said polymer is about 1:1 to about 1:6, for example a 1:3 ratio, spray dried from a mixture of one of chloroform or dichloromethane and one of methanol or ethanol, preferably dichloromethane/ethanol in a 1:1 ratio.

The invention also provides solid dosage forms comprising the solid solution described above. Solid dosage forms include tablets, capsules and chewable tablets. Known excipients can be blended with the solid solution to provide the desired dosage form. For example, a capsule can contain the solid solution blended with (a) a disintegrant and a lubricant, or (b) a disintegrant, a lubricant and a surfactant. In addition a capsule can also contain a bulking agent, such as e.g. lactose or microcrystalline cellulose. A tablet can contain the solid solution blended with at least one disintegrant, a lubricant, a surfactant, and a glidant. A chewable tablet can contain the solid solution blended with a bulking agent, a lubricant, and if desired an additional sweetening agent (such as an artificial sweetener), and suitable flavours.

The pharmaceutical formulations may be presented to a patient in “patient packs” containing an entire course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

Compositions for topical use and nasal delivery include ointments, creams, sprays, patches, gels, liquid drops and inserts (for example intraocular inserts). Such compositions can be formulated in accordance with known methods.

Compositions for parenteral administration are typically presented as sterile aqueous or oily solutions or fine suspensions, or may be provided in finely divided sterile powder form for making up extemporaneously with sterile water for injection.

Examples of formulations for rectal or intra-vaginal administration include pessaries and suppositories which may be, for example, formed from a shaped moldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compounds of the invention will generally be presented in unit dosage form and, as such, will typically contain sufficient compound to provide a desired level of biological activity. For example, a formulation may contain from 1 nanogram to 2 grams of active ingredient, e.g. from 1 nanogram to 2 milligrams of active ingredient. Within this range, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 milligrams to 1 gram, of active compound.

The active compound will be administered to a patient in need thereof (for example a human or animal patient) in an amount sufficient to achieve the desired therapeutic effect.

Methods of Treatment

The compound of the invention will be useful in the prophylaxis or treatment of a range of disease states or conditions mediated by cyclin dependent kinases and glycogen synthase kinase-3. Examples of such disease states and conditions are set out above.

The compound is generally administered to a subject in need of such administration, for example a human or animal patient, preferably a human.

The compound is typically administered in amounts that are therapeutically or prophylactically useful and which generally are non-toxic. However, in certain situations (for example in the case of life threatening diseases), the benefits of administering a compound of the invention may outweigh the disadvantages of any toxic effects or side effects, in which case it may be considered desirable to administer compounds in amounts that are associated with a degree of toxicity.

The compound may be administered over a prolonged term to maintain beneficial therapeutic effects or may be administered for a short period only. Alternatively the compound may be administered in a continuous manner or in a manner that provides persistent intermittent dosing (e.g. a pulsatile manner).

A typical daily dose of the compound of formula (I) can be in the range from 100 picograms to 100 milligrams per kilogram of body weight, more typically 5 nanograms to 25 milligrams per kilogram of bodyweight, and more usually 10 nanograms to 15 milligrams per kilogram (e.g. 10 nanograms to 10 milligrams, and more typically 1 microgram per kilogram to 20 milligrams per kilogram, for example 1 microgram to 10 milligrams per kilogram) per kilogram of bodyweight although higher or lower doses may be administered where required. The compound of the formula (1) can be administered on a daily basis or on a repeat basis every 2, or 3, or 4, or 5, or 6, or 7, or 10 or 14, or 21, or 28 days for example.

The compound of the invention may be administered orally in a range of doses, for example 1 to 1500 mg, 2 to 800 mg, or 5 to 500 mg, e.g. 2 to 200 mg or 10 to 1000 mg, particular examples of doses including 10, 20, 50 and 80 mg. The compound may be administered once or more than once each day. The compound can be administered continuously (i.e. taken every day without a break for the duration of the treatment regimen). Alternatively, the compound can be administered intermittently, i.e. taken continuously for a given period such as a week, then discontinued for a period such as a week and then taken continuously for another period such as a week and so on throughout the duration of the treatment regimen. Examples of treatment regimens involving intermittent administration include regimens wherein administration is in cycles of one week on, one week off; or two weeks on, one week off; or three weeks on, one week off; or two weeks on, two weeks off; or four weeks on, two weeks off; or one week on, three weeks off—for one or more cycles, e.g. 2, 3, 4, 5, 6, 7, 8, 9 or 10 or more cycles.

Ultimately, however, the quantity of compound administered and the type of composition used will be commensurate with the nature of the disease or physiological condition being treated and will be at the discretion of the physician.

The compounds of formula (I) and sub-groups as defined herein can be administered as the sole therapeutic agent or they can be administered in combination therapy with one of more other compounds for treatment of a particular disease state, for example a neoplastic disease such as a cancer as hereinbefore defined. Examples of other therapeutic agents or therapies that may be administered or used together (whether concurrently or at different time intervals) with the compounds of the invention include but are not limited to topoisomerase inhibitors, alkylating agents, antimetabolites, DNA binders, microtubule inhibitors (tubulin targeting agents), monoclonal antibodies and signal transduction inhibitors, particular examples being cisplatin, cyclophosphamide, doxorubicin, irinotecan, fludarabine, 5FU, taxanes, mitomycin C and radiotherapy.

The compounds as defined herein can be administered as the sole therapeutic agent or they can be administered in combination therapy with one of more other compounds for treatment of a particular disease state, for example a neoplastic disease such as a cancer as hereinbefore defined. Examples of other therapeutic agents or treatments that may be administered together (whether concurrently or at different time intervals) with the compounds of the formula (I) include but are not limited to:
Topoisomerase I inhibitors
Antimetabolites
Tubulin targeting agents
DNA binder and topoisomerase II inhibitors
Alkylating Agents
Monoclonal Antibodies.
Anti-Hormones
Signal Transduction Inhibitors
Proteasome Inhibitors
DNA methyl transferases
Cytokines and retinoids
Chromatin targeted therapies
Radiotherapy, and,
Other therapeutic or prophylactic agents; for example agents that reduce or alleviate some of the side effects associated with chemotherapy. Particular examples of such agents include anti-emetic agents and agents that prevent or decrease the duration of chemotherapy-associated neutropenia and prevent complications that arise from reduced levels of red blood cells or white blood cells, for example erythropoietin (EPO), granulocyte macrophage-colony stimulating factor (GM-CSF), and granulocyte-colony stimulating factor (G-CSF). Also included are agents that inhibit bone resorption such as bisphosphonate agents e.g. zoledronate, pamidronate and ibandronate, agents that suppress inflammatory responses (such as dexamethazone, prednisone, and prednisolone) and agents used to reduce blood levels of growth hormone and IGF-I in acromegaly patients such as synthetic forms of the brain hormone somatostatin, which includes octreotide acetate which is a long-acting octapeptide with pharmacologic properties mimicking those of the natural hormone somatostatin. Further included are agents such as leucovorin, which is used as an antidote to drugs that decrease levels of folic acid, or folinic acid it self and agents such as megestrol acetate which can be used for the treatment of side-effects including oedema and thromoembolic episodes.

For the case of CDK inhibitors combined with other therapies, the two or more treatments may be given in individually varying dose schedules and via different routes.

Where the compound of the formula (I) is administered in combination therapy with one, two, three, four or more other therapeutic agents (preferably one or two, more preferably one), the compounds can be administered simultaneously or sequentially. When administered sequentially, they can be administered at closely spaced intervals (for example over a period of 5-10 minutes) or at longer intervals (for example 1, 2, 3, 4 or more hours apart, or even longer periods apart where required), the precise dosage regimen being commensurate with the properties of the therapeutic agent(s).

The compounds of the invention may also be administered in conjunction with non-chemotherapeutic treatments such as radiotherapy, photodynamic therapy, gene therapy; surgery and controlled diets.

For use in combination therapy with another chemotherapeutic agent, the compound of the formula (I) and one, two, three, four or more other therapeutic agents can be, for example, formulated together in a dosage form containing two, three, four or more therapeutic agents. In an alternative, the individual therapeutic agents may be formulated separately and presented together in the form of a kit, optionally with instructions for their use.

A person skilled in the art would know through his or her common general knowledge the dosing regimes and combination therapies to use.

Methods of Diagnosis

Prior to administration of a compound of the formula (I), a patient may be screened to determine whether a disease or condition from which the patient is or may be suffering is one which would be susceptible to treatment with a compound having activity against cyclin dependent kinases.

For example, a biological sample taken from a patient may be analysed to determine whether a condition or disease, such as cancer, that the patient is or may be suffering from is one which is characterised by a genetic abnormality or abnormal protein expression which leads to over-activation of CDKs or to sensitisation of a pathway to normal CDK activity. Examples of such abnormalities that result in activation or sensitisation of the CDK2 signal include up-regulation of cyclin E, (Harwell R M, Mull B B, Porter D C, Keyomarsi K.; J Biol Chem. 2004 Mar. 26;279(13):12695-705) or loss of p21 or p27, or presence of CDC4 variants (Rajagopalan H, Jallepalli P V, Rago C, Velculescu V E, Kinzler K W, Vogelstein B, Lengauer C.; Nature. 2004 Mar. 4;428(6978):77-81). Tumours with mutants of CDC4 or up-regilation, in particular over-expression, of cyclin E or loss of p21 or p27 may be particularly sensitive to CDK inhibitors. The term up-regulation includes elevated expression or over-expression, including gene amplification (i.e. multiple gene copies) and increased expression by a transcriptional effect, and hyperactivity and activation, including activation by mutations.

Thus, the patient may be subjected to a diagnostic test to detect a marker characteristic of up-regulation of cyclin E, or loss of p21 or p27, or presence of CDC4 variants. The term diagnosis includes screening. By marker we include genetic markers including, for example, the measurement of DNA composition to identify mutations of CDC4. The term marker also includes markers which are characteristic of up regulation of cyclin E, including enzyme activity, enzyme levels, enzyme state (e.g. phosphorylated or not) and mRNA levels of the aforementioned proteins. Tumours with upregulation of cyclin E, or loss of p21 or p27 may be particularly sensitive to CDK inhibitors. Tumours may preferentially be screened for upregulation of cyclin E, or loss of p21 or p27 prior to treatment. Thus, the patient may be subjected to a diagnostic test to detect a marker characteristic of up-regulation of cyclin E, or loss of p21 or p27.

The diagnostic tests are typically conducted on a biological sample selected from tumour biopsy samples, blood samples (isolation and enrichment of shed tumour cells), stool biopsies, sputum, chromosome analysis, pleural fluid, peritoneal fluid, or urine.

It has been found, Rajagopalan et al (Nature. 2004 Mar. 4;428(6978):77-81), that there were mutations present in CDC4 (also known as Fbw7 or Archipelago) in human colorectal cancers and endometrial cancers (Spruck et al, Cancer Res. 2002 Aug. 15;62(16):4535-9). Identification of individual carrying a mutation in CDC4 may mean that the patient would be particularly suitable for treatment with a CDK inhibitor. Tumours may preferentially be screened for presence of a CDC4 variant prior to treatment. The screening process will typically involve direct sequencing, oligonucleotide microarray analysis, or a mutant specific antibody.

Methods of identification and analysis of mutations and up-regulation of proteins are well known to a person skilled in the art. Screening methods could include, but are not limited to, standard methods such as reverse-transcriptase polymerase chain reaction (RT-PCR) or in-situ hybridisation.

In screening by RT-PCR, the level of mRNA in the tumour is assessed by creating a cDNA copy of the mRNA followed by amplification of the cDNA by PCR. Methods of PCR amplification, the selection of primers, and conditions for amplification, are known to a person skilled in the art. Nucleic acid manipulations and PCR are carried out by standard methods, as described for example in Ausubel, F. M. et al., eds. Current Protocols in Molecular Biology, 2004, John Wiley & Sons Inc., or Innis, M. A. et-al., eds. PCR Protocols: a guide to methods and applications, 1990, Academic Press, San Diego. Reactions and manipulations involving nucleic acid techniques are also described in Sambrook et al., 2001, 3^{rd} Ed, Molecular Cloning: A Laboratory Manual, Cold Spring Harbor Laboratory Press. Alternatively a commercially available kit for RT-PCR (for example Roche Molecular Biochemicals) may be used, or methodology as set forth in U.S. Pat. Nos. 4,666,828; 4,683,202; 4,801,531; 5,192,659, 5,272,057, 5,882,864, and 6,218,529 and incorporated herein by reference.

An example of an in-situ hybridisation technique for assessing mRNA expression would be fluorescence in-situ hybridisation (FISH) (see Angerer, 1987 Meth. Enzymol., 152: 649).

Generally, in situ hybridization comprises the following major steps: (1) fixation of tissue to be analyzed; (2) prehybridization treatment of the sample to increase accessibility of target nucleic acid, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions. Standard methods for carrying out FISH are described in Ausubel, F. M. et al., eds. Current Protocols in Molecular Biology, 2004, John Wiley & Sons Inc and Fluorescence In Situ Hybridization: Technical Overview by John M. S. Bartlett in Molecular Diagnosis of Cancer, Methods and Protocols, 2nd ed.; ISBN: 1-59259-760-2; March 2004, pps. 077-088; Series: Methods in Molecular Medicine.

Alternatively, the protein products expressed from the mRNAs may be assayed by immunohistochemistry of tumour samples, solid phase immunoassay with microtiter plates, Western blotting, 2-dimensional SDS-polyacrylamide gel electrophoresis, ELISA, flow cytometry and other methods known in the art for detection of specific proteins. Detection methods would include the use of site specific antibodies. The skilled person will recognize that all such well-known techniques for detection of upregulation of cyclin E, or loss of p21 or p27, or detection of CDC4 variants could be applicable in the present case.

Therefore, all of these techniques could also be used to identify tumours particularly suitable for treatment with the compounds of the invention.

Tumours with mutants of CDC4 or up-regulation, in particular over-expression, of cyclin E or loss of p21 or p27 may be particularly sensitive to CDK inhibitors. Tumours may preferentially be screened for up-regulation, in particular over-expression, of cyclin E (Harwell R M, Mull B B, Porter D C, Keyomarsi K.; J Biol Chem. 2004 Mar. 26;279(13):12695-705) or loss of p21 or p27 or for CDC4 variants prior to treatment (Rajagopalan H, Jallepalli P V, Rago C, Velculescu V E, Kinzler K W, Vogelstein B, Lengauer C.; Nature. 2004 Mar. 4;428(6978):77-81).

Patients with mantle cell lymphoma (MCL) could be selected for treatment with a compound of the invention using diagnostic tests outlined herein. MCL is a distinct clinicopathologic entity of non-Hodgkin's lymphoma, characterized by proliferation of small to medium-sized lymphocytes with co-expression of CD5 and CD20, an aggressive and incurable clinical course, and frequent t(11;14)(q13;q32) translocation. Over-expression of cyclin D1 mRNA, found in mantle cell lymphoma (MCL), is a critical diagnostic marker. Yatabe et al (Blood. 2000 Apr. 1;95(7):2253-61) proposed that cyclin D1-positivity should be included as one of the standard criteria for MCL, and that ilmovative therapies for this incurable disease should be explored on the basis of the new criteria. Jones et al (J Mol Diagn. 2004 May;6(2):84-9) developed a real-time, quantitative, reverse transcription PCR assay for cyclin D1 (CCND1) expression to aid in the diagnosis of mantle cell lymphoma (MCL). Howe et al (Clin Chem. 2004 January;50(1):80-7) used real-time quantitative RT-PCR to evaluate cyclin D1 mRNA expression and found that quantitative RT-PCR for cyclin D1 mRNA normalized to CD19 mRNA can be used in the diagnosis of MCL in blood, marrow, and tissue. Alternatively, patients with breast cancer could be selected for treatment with a CDK inhibitor using diagnostic tests outline above. Tumour cells commonly overexpress cyclin E and it has been shown that cyclin E is over-expressed in breast cancer (Harwell et al, Cancer Res, 2000, 60, 481-489). Therefore breast cancer may in particular be treated with a CDK inhibitor as provided herein.

### BRIEF DESCRIPTION OF THE DRAWINGS

FIG. 1 is a depiction of the three dimensional structure of 4-(2,6-dichloro-benzoylamino)-1H-pyrazole-3-carboxylic acid (1-methanesulphonyl-piperidin-4-yl)-amide as determined by a single crystal X-ray diffraction study.

FIG. 2 is graphical representation of the structure generated by an X-ray diffraction study 4-(2,6-dichloro-benzoylamino)-1H-pyrazole-3-carboxylic acid (1-methanesulphonyl-piperidin-4-yl)-amide.

FIG. 3 is an X-ray powder diffractogram of 4-(2,6-dichloro-benzoylamino)-1H-pyrazole-3-carboxylic acid (1-methanesulphonyl-piperidin-4-yl)-amide.

FIG. 4 is a DSC scan of a crystalline form of 4-(2,6-dichloro-benzoylamino)-1H-pyrazole-3-carboxylic acid (1-methanesulphonyl-piperidin-4-yl)-amide.

FIG. 5 is a weight loss profile obtained by thermogravimetric analysis of a crystalline form of 4-(2,6-dichloro-benzoylamino)-1H-pyrazole-3-carboxylic acid (1-methanesulphonyl-piperidin-4-yl)-amide.

FIG. 6 is a vapour sorption/desorption profile of a crystalline form of 4-(2,6-dichloro-benzoylamino)-1H-pyrazole-3-carboxylic acid (1-methanesulphonyl-piperidin-4-yl)-amide.

FIG. 7 is a graph of solubility against time for several formulations containing a solid dispersion of 4-(2,6-dichloro-benzoylamino)-1H-pyrazole-3-carboxylic acid (1-methanesulphonyl-piperidin-4-yl)-amide and PVP, where (1) indicates the non-encapsulated solid dispersion of PVP and the compound of formula (I) containing no further excipients; (2) indicates the solid dispersion (1) packed tightly into a size 0 capsule and (3) indicates the formulated sample.

### EXAMPLES

The invention will now be illustrated, but not limited, by reference to the specific embodiments described in the following examples.

### Example 1

### Synthesis of 4-(2,6-dichloro-benzoylamino)-1H-pyrazole-3-carboxylic acid (1-methanesulphonyl-piperidin-4-yl)-amide and crystals thereof

The compound 4-(2,6-dichloro-benzoylamino)-1H-pyrazole-3-carboxylic acid (1-methanesulphonyl-piperidin-4-yl)-amide can be prepared by the synthetic sequence illustrated in Scheme 1 above and described in more detail below.

### Stage 1: Preparation of 4-nitro-1H-pyrazole-3-carboxylic acid methyl ester

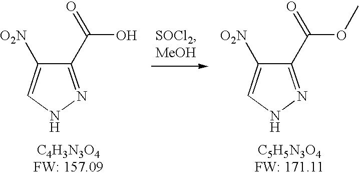

4-Nitro-1H-pyrazole-3-carboxylic acid (1.350 Kg, 8.59 Mol, 1.0 wt) and methanol (10.80 L, 8.0 vol) were charged to a flange flask equipped with a mechanical stirrer, condenser and thermometer. The suspension was cooled to 0 to 5° C. under nitrogen and thionyl chloride (0.702 L, 9.62 Mol, 0.52 vol) added at this temperature. The mixture was warmed to 15 to 25° C. over 16 to 24 hours. Reaction completion was determined by ¹H NMR analysis (d₆-DMSO). The mixture was concentrated under vacuum at 35 to 45° C. and toluene (2.70 L, 2.0 vol) charged to the residue and removed under vacuum at 35 to 45° C. The toluene azeotrope was repeated twice using toluene (2.70 L, 2.0 vol) to give 4-nitro-1H-pyrazole-3-carboxylic acid methyl ester [1.467 Kg, 99.8% th, 108.7% w/w, ¹H NMR (d₆-DMSO) concordant with structure, no entrained solvent] as an off-white solid.

### Stage 2: Preparation of 4-amino-1H-pyrazole-3-carboxylic acid methyl ester

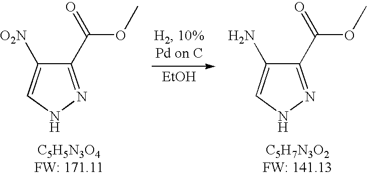

A suspension of 4-nitro-1H-pyrazole-3-carboxylic acid methyl ester (1.467 Kg, 8.57 Mol, 1.0 wt) and ethanol (14.70 L, 10.0 vol) was heated to and maintained at 30 to 35° C. until complete dissolution occurred. 10% Palladium on carbon (10% Pd/C wet paste, 0.205 Kg, 0.14 wt) was charged to a separate flask under nitrogen and a vacuum/nitrogen purge cycle performed (×3). The solution of 4-nitro-1H-pyrazole-3-carboxylic acid methyl ester in ethanol was charged to the catalyst and the vacuum/nitrogen purge cycle repeated (×3). A vacuum/hydrogen purge cycle was performed (×3) and the reaction placed under an atmosphere of hydrogen. The reaction mixture was stirred at 28 to 30° C. until deemed complete by ¹H NMR analysis (d₆-DMSO). The mixture was filtered under nitrogen and concentrated under vacuum at 35 to 45° C. to give 4-amino-1H-pyrazole-3-carboxylic acid methyl ester [1.184 Kg, 97.9% th, 80.7% w/w, ¹H NMR (d₆-DMSO) concordant with structure, corrected for 0.27% w/w entrained ethanol] as an off-white solid.

### Stage 3: Preparation of 4-(2,6-dichlorobenzoylamino)-1H-pyrazole-3-carboxylic acid methyl ester

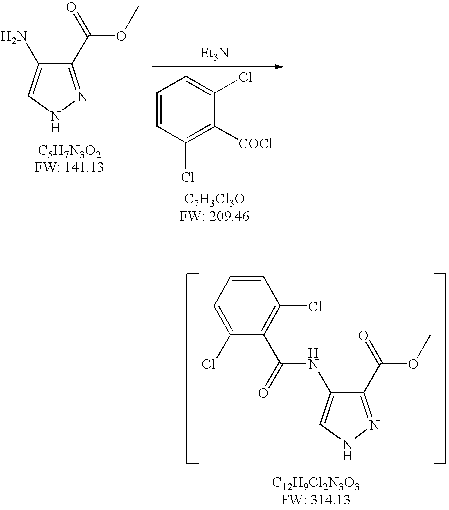

Triethylamine (1.42 L, 10.20 Mol, 1.2 vol) was added to solution of 4-amino-1H-pyrazole-3-carboxylic acid methyl ester (1.184 Kg, 8.39 Mol, 1.0 wt) in 1,4-dioxane (10.66 L, 9.0 vol) at 15 to 25° C. under nitrogen. 2,6-Dichlorobenzoyl chloride (1.33 L, 9.28 Mol, 1.12 vol) was charged at 15 to 25° C. followed by a line rinse of 1,4-dioxane (1.18 L, 1.0 vol) and the reaction mixture stirred at 15 to 25° C. for 14 to 24 hours. Reaction completion was determined by 1HNMR analysis¹. The reaction mixture was filtered, the filter-cake washed with 1,4-dioxane (2×1.18 L, 2×1.0 vol) and the combined filtrates progressed to Stage 4 without further isolation. ¹ A sample of the reaction mixture was filtered, the filtrates dissolved in d₆-DMSO and a 1H NMR spectrum obtained

### Stage 4: Preparation of 4-(2,6-dichlorobenzoylamino)-1H-pyrazole-3-carboxylic acid

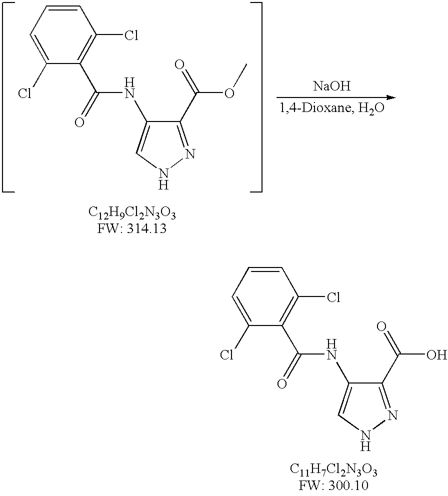

A solution of 4-(2,6-dichlorobenzoylamino)-1H-pyrazole-3-carboxylic acid methyl ester (1.308 Kg, 4.16 Mol, 1.0 wt) in 1,4-dioxane (6.47 L, 5.0 vol) was charged, in one portion, to 2M aq. sodium hydroxide solution (7.19 L, 14.38 Mol, 5.5 vol) at 35 to 45° C. The reaction mixture was cooled to 15 to 25° C. over 14 to 24 hours.

Reaction completion was determined by TLC analysis². The reaction mixture was concentrated under vacuum at 45 to 50° C. The resultant oily residue was diluted with water (11.77 L, 9.0 vol) and acidified to pH1 with conc. aq. hydrochloric acid at 15 to 30° C. The precipitate was collected by filtration, washed with water (5.88 L, 4.5 vol), pulled dry on the filter and a displacement wash with heptanes (5.88 L, 4.5 vol) added. The filter-cake was charged to a 20 L rotary evaporator flask and azeo-dried with toluene (2×5.23 L, 2×4.0 vol) to afford 4-(2,6-dichlorobenzoylamino)-1H-pyrazole-3-carboxylic acid [1.207 Kg, 96.6% th, 92.3% w/w, ¹H NMR (d₆-DMSO) concordant with structure, 98.31% by HPLC area] as a yellow solid. ² Eluant: Ethyl acetate. UV visualisation. R_{f ester} 0.5, R_{f Stage 4} 0.0

### Stage 5: Preparation of 4-{[4-(2,6-dichlorobenzoylamino)-1H-pyrazole-3-carbonyl]amino}-piperidine-1-carboxylic acid tert-butyl ester

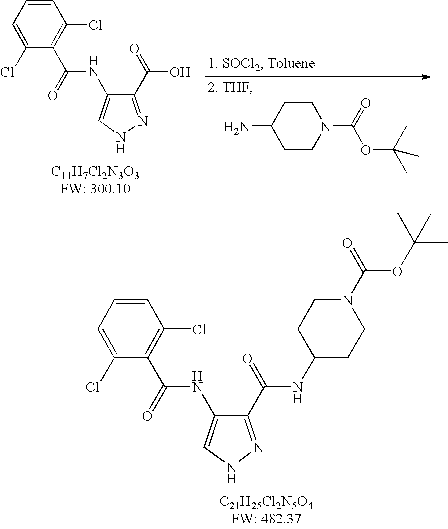

Thionyl chloride (0.25 L, 3.43 Mol, 0.3 vol) was added to a stirred suspension of 4-(2,6-dichlorobenzoylamino)-1H-pyrazole-3-carboxylic acid (0.806 Kg, 2.69 Mol, 1.0 wt) in toluene (8.00 L, 10.0 vol) under nitrogen at 16 to 25° C. The contents were then heated to and stirred at 80 to 100° C. for 16 to 24 hours. Reaction completion was determined by ¹H NMR analysis. The reaction mixture was cooled to 40 to 50° C., concentrated to dryness under vacuum at 45 to 50° C. and the residue azeo-dried with toluene (3×1.60 L, 3×2.0 vol) under vacuum at 45 to 50° C. to afford a white solid. The solid was transferred to a suitable vessel, tetrahydrofuran (4.00 L, 5.0 vol) charged, the contents stirred under nitrogen and triethylamine (0.42 L, 3.01 Mol, 0.512 vol) added at 16 to 25° C. A solution of 4-aminopiperidine-1-carboxylic acid tert-butyl ester (0.569 Kg, 2.84 Mol, 0.704 wt) in tetrahydrofuran (4.00 L, 5.0 vol) was then added to the reaction flask at 16 to 30° C. and the reaction mixture heated to and stirred at 45 to 50° C. for 2 to 16 hours. Reaction completion was determined by ¹H NMR analysis. The reaction mixture was cooled to 16 to 25° C. and quenched with water (4.00 L, 5.0 vol) and mixed heptanes (0.40 L, 0.5 vol). The contents were stirred for up to 10 minutes, the layers separated and the aqueous phase extracted with tetrahydrofuran:mixed heptanes [(9:1), 3×4.00 L, 3×5.0 vol]. The combined organic phases were washed with water (1.81 L, 2.5 vol) and concentrated under vacuum at 40 to 45° C. The residue was azeo-dried with toluene (3×4.00 L, 3×5.0 vol) to yield crude 4-{[4-(2,6-dichlorobenzoylamino)-1H-pyrazole-3-carbonyl]amino}-piperidine-1-carboxylic acid tert-butyl ester (1.257 Kg, 97.1% th, 156.0% w/w, corrected for 0.90% w/w entrained solvent). Several batches of compound were prepared in this way and the batches were combined for purification.

Crude 4-{[4-(2,6-dichlorobenzoylamino)-1H-pyrazole-3-carbonyl]amino}-piperidine-1-carboxylic acid tert-butyl ester (5.22 Mol, 1.0 wt), toluene (12.00 L, 4.87vol) and methanol (0.30 L, 0.13 vol) were stirred under nitrogen for 3 to 18 hours at 16 to 25° C. The solid was isolated by filtration, the filter-cake washed with toluene (2×1.60 L, 2×0.7 vol) and dried under vacuum at 40 to 50° C. to yield 4- {[4-(2,6-dichlorobenzoylamino)-1H-pyrazole-3-carbonyl]amino}-piperidine-1-carboxylic acid tert-butyl ester [2.242 Kg, 86.6% th, 139.2% w/w, ¹H NMR (d₆-DMSO) concordant, 99.41% by HPLC area] as an off-white solid.

### Stage 6: Preparation of 4-(2,6-dichlorobenzoylamino)-1H-pyrazole-3-carboxylic acid -piperidin-4-ylamide methanesulphonate

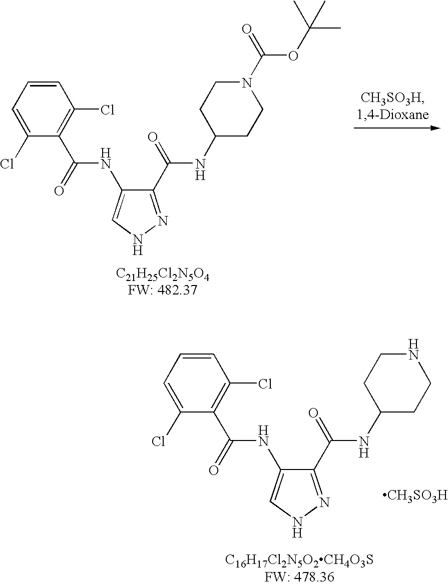

4-{[4-(2,6-Dichlorobenzoylamino)-1H-pyrazole-3-carbonyl]amino}-piperidine-1-carboxylic acid tert-butyl ester (0.561 Kg, 1.16 Mol, 1.0 wt) and 1,4-dioxane (14.00 L, 26.0 vol) were stirred under nitrogen and heated to 80 to 90° C. Methanesulphonic acid (0.30 L, 4.62 Mol, 0.54 vol) was added over 30 to 60 minutes at 80 to 90° C. and the contents heated to and maintained at 95 to 105° C. for 1 to 24 hours. Reaction completion was determined by ¹H NMR analysis. The reaction mixture was cooled to 20 to 30° C. and the resulting precipitate collected by filtration. The filter-cake was washed with propan-2-ol (2×1.10 L, 2×2.0 vol) and pulled dry on the filter for 3 to 24 hours to give 4-(2,6-dichlorobenzoylamino)-1H-pyrazole-3-carboxylic acid piperidin-4-ylamide methanesulphonate [0.558 Kg, 100.2% th, 99.4% w/w, ¹H NMR (d₆-DMSO) concordant with structure, 98.13% by HPLC area] as an off-white solid.

### Stage 7: Preparation of 4-(2,6-dichlorobenzoylamino)-1H-pyrazole-3-carboxylic acid (1-methanesulphonyl-piperidin-4-yl)-amide

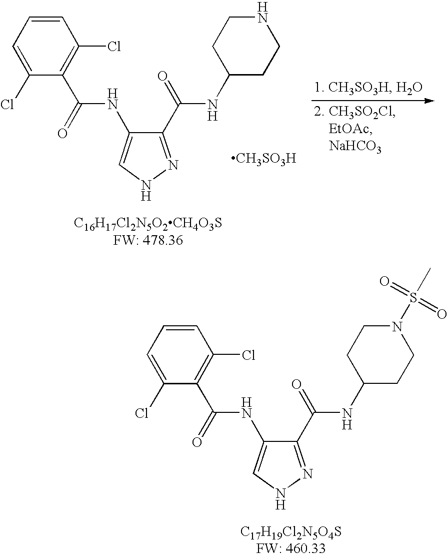

Methanesulphonic acid (0.055 L, 0.85 Mol, 0.1 vol) was added to a stirred suspension of 4-(2,6-dichlorobenzoylamino)-1H-pyrazole-3-carboxylic acid piperidin-4-ylamide methanesulphonate (0.562 Kg, 1.17 Mol, 1.0 wt) in water (5.60 L, 10.0 vol) at 15 to 40° C. The reaction mixture was heated to and stirred at 95 to 105° C. for 80 to 100 minutes. Reaction completion was determined by HPLC analysis. The mixture was cooled to 15 to 20° C., sodium hydrogen carbonate (1.224 Kg, 14.57 Mol, 2.18 wt) charged at 15 to 25° C. followed by ethyl acetate (4.20 L, 7.5 vol) and the temperature adjusted to 15 to 25° C. as necessary. Methanesulphonyl chloride (0.455 L, 5.88 Mol, 0.81 vol) was added in five aliquots over 120 to 180 minutes at 15 to 25° C. and the reaction mixture stirred for a further 30 to 45 minutes. Reaction completion was determined by HPLC analysis. The ethyl acetate was removed under vacuum at 35 to 45° C., the resulting slurry filtered, the filter-cake washed with water (0.56 L, 1.0 vol) and transferred to a suitably sized flask. Water (2.81 L, 5.0 vol) was charged and the mixture stirred for 30 to 40 minutes at 15 to 25° C. then filtered, the filter-cake washed with water (056 L, 1.0 vol) and pulled dry on the pad for 1 to 24 hours. The collected solids were dried under vacuum at 40 to 50° C. to give crude 4-(2,6-dichlorobenzoylamino)-1H-pyrazole-3-carboxylic acid (1-methanesulphonyl-piperidin-4-yl)-amide [0.490 Kg, 90.7% th, 87.2% w/w, ¹H NMR (d₆-DMSO) concordant with structure, 98.05% by HPLC area] as an off-white solid.

### Stage 8: Recrystallisation of 4-(2,6-dichlorobenzoylamino)-1H-pyrazole-3-carboxylic acid (1-methanesulphonyl-piperidin-4-yl)-amide

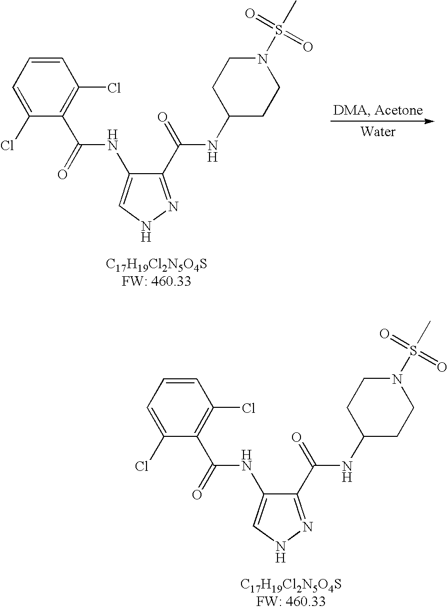

Crude 4-(2,6-dichlorobenzoylamino)-1H-pyrazole-3-carboxylic acid (1-methanesulphonyl-piperidin-4-yl)-amide (5.506 Kg, 11.96 Mol, 1.0 wt), N,N-dimethylacetamide (8.00 L, 1.5 vol) and acetone (11.00 L, 2.0 vol) were stirred under nitrogen and heated to 40 to 50° C. The resulting solution was clarified by filtration through glass microfibre paper and the filtrates heated to 60 to 80° C. Water (10.50 L, 2.0 vol) was added at 60 to 80° C. such that reflux was maintained throughout. The mixture was cooled to and aged at 15 to 25° C. for 14 to 24 hours, the crystallised solid isolated by filtration, the filter-cake washed with water (6.00 L, 1.0 vol) and transferred to a suitable vessel. Water (11.00 L, 2.0 vol) was charged, the mixture stirred for 30 to 40 minutes at 15 to 25° C. and then filtered. The filter-cake was washed with water (6.00 L, 1.0 vol) and pulled dry on the filter for at least 30 minutes. The solid was dried under vacuum at 40 to 50° C. to yield 4-(2,6-dichlorobenzoylamino)-1H-pyrazole-3-carboxylic acid (1-methanesulphonyl-piperidin-4-yl)-amide [4.530 Kg, 82.3% th, 82.3% w/w, ¹H NMR (d₆-DMSO) concordant with structure, 99.29% by HPLC area] as a white solid.

### Example 2

### Alternative Synthesis of 4-(2,6-dichloro-benzoylamino)-1H-pyrazole-3-carboxylic acid (1-methanesulphonyl-piperidin-4-yl)-amide

### Step 1: Synthesis of 4-[(4-nitro-1H-pyrazole-3-carbonyl)-amino]-piperidine-1-carboxylic acid tert-butyl ester

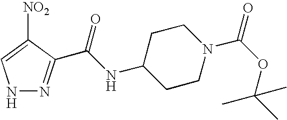

4-Nitropyrazole-3-carboxylic acid (20.0 g, 127.4 mmol) was suspended in CH₂Cl₂/DMF (99:1, 400 mL), treated cautiously with oxalyl chloride (11.6 mL, 134 mmol) and then stirred at room temperature for 16 h. The reaction mixture was evaporated then re-evaporated with toluene (×3) to give a yellow solid. The resultant acid chloride was suspended in dioxane (400 mL), treated with triethylamine (26.4 mL, 190 mmol) followed by 4-amino-1-BOC-piperidine (25.0 g, 125 mmol) and stirred at room temperature for 6 h. The reaction mixture was filtered and the solid collected stirred in water (500 mL) and then re-filtered. The solid collected was dried in vacuo, azeotroping with toluene, to give the title compound (37.6 g).

### Step 2: Synthesis of 4-nitro-1H-pyrazole-3-carboxylic acid piperidin-4-ylamide

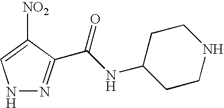

4-[(4-Nitro-1H-pyrazole-3-carbonyl)-amino]-piperidine-1-carboxylic acid tert-butyl ester (20.0 g, 59.0 mmol) was suspended in dioxane-CH₂Cl₂ (1:1, 400 ml) and treated with 4M HCl in dioxane (100 mL). The mixture was stirred at room temperature for 16 h and the solid formed collected by filtration, and dried in vacuo to give the title compound as a white solid (13.8 g).

### Step 3: Synthesis of 4-nitro-1H-pyrazole-3-carboxylic acid (1-methanesulphonyl-piperidin-4-yl)-amide

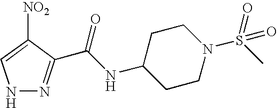

To a suspension of 4-nitro-1H-pyrazole-3-carboxylic acid piperidin-4-ylamide (13.7 g, 50.0 mmol) in dioxane-acetonitrile (1:1, 250 mL) was added triethylamine (17.4 mL, 125 mmol) followed by methanesulphonyl chloride (4.26 mL, 55.0 mmol). The mixture was stirred at 45° C. for 5 h then reduced in vacuo. To the residue was added water (500 mL), the mixture stirred for 20 min and the solid collected by filtration and dried in vacuo, azeotroping with toluene (×3), to give the title compound as an off-white solid (12.8 g)

### Step 4: Synthesis of 4-amino-1H-pyrazole-3-carboxylic acid (1-methanesulphonyl-piperidin-4-yl)-amide

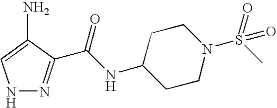

4-Nitro-1H-pyrazole-3-carboxylic acid (1-methanesulphonyl-piperidin-4-yl)-amide (5.0 g) was dissolved in DMF (30 mL), treated with 10% palladium on carbon (0.5 g) then hydrogenated at room temperature and 45 psi until the reaction was complete. The reaction mixture was filtered through Celite and reduced in vacuo. The residue was triturated with water (200 mL) and the resultant solid collected by filtration and dried in vacuo, azeotroping with toluene (×3) to give the title compound as the major product (3.5 g)

### Step 5: Synthesis of 4-(2,6-dichloro-benzoylamino)-1H-pyrazole-3-carboxylic acid (1-methanesulphonyl-piperidin-4-yl)-amide

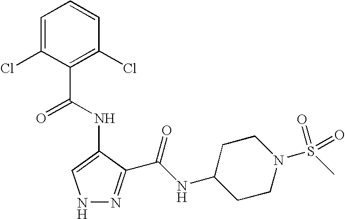

To a mixture of 4-amino-1H-pyrazole-3-carboxylic acid (1-methanesulphonyl-piperidin-4-yl)-amide (3.4 g, ˜10 mmol) and triethylamine (1.53 mL, 11 mmol) in dioxane (50 mL) at 45° C. was slowly added 2,6-dichlorobenzoyl chloride (1.4 mL, 10 mmol). The mixture was heated at 45° C. for 2 h, poured into water (250 mL) and then extracted with EtOAc (2×200 mL). The combined organic extracts were reduced in vacuo and purified by column chromatography on silica gel eluting with P.E-EtOAc (1:0-0:1). The product containing fractions were reduced in vacuo and the residue taken up in 2M aqueous NaOH-MeOH (1:1, 50 mL) and stirred at ambient temperature for 2 h. The MeOH was removed in vacuo and the mixture extracted with EtOAc. The organic portion was washed with brine, dried over MgSO₄ and reduced in vacuo. The residue was purified by hot slurry with EtOH to give the title compound as an off-white solid (2.52 g).

### Example 3

### Determination of the crystal structure of 4-(2,6-dichlorobenzoylamino)-1H-pyrazole-3-carboxylic acid (1-methanesulphonyl-piperidin-4-yl)-amide by X-ray diffraction

A crystal was obtained by evaporation of a CHCl₃ solution of the compound 4-(2,6-dichlorobenzoylamino)-1H-pyrazole-3-carboxylic acid (1-methanesulphonyl-piperidin-4-yl)-amide prepared as described in Example 2.

The crystal used for the diffraction experiment was colourless and of irregular shape with dimensions 0.15×015×0.04 mm³. Crystallographic data were collected at 104 K using CuKα radiation (λ=1.5418 Å) from a Rigaku rotating anode RU3HR, Osmic blue confocal optics, AFC9 ¼χ goniometer and a Rigaku Jupiter CCD detector. Images were collected in three ω scans at 2θ=15° and four scans at 2θ=90° with a detector to crystal distance of 67 mm. Data collection was controlled by CrystalClear software and images were processed and scaled by Dtrek. Due to a high absorption coefficient (μ=4.04 mm⁻¹) data had to be corrected using 4^{th} order Fourier absorption correction. It was found that the crystals belong to a monoclinic space group C2/c (#15) with crystal lattice parameters a=9.15, b=31.32, c=7.93 Å, β=113.3°, α=γ=90°. One short room temperature scan was taken to check crystal lattice parameters and symmetry. It was found that symmetry is the same as at 104 K and crystal lattice parameters are similar (room temperature a=9.19, b=31.31, c=8.09 Å, β=115.2°). The unit cell dimensions a, b & c have a deviation (s.u., standard uncertainty) of 5%.

The crystal structure was solved using direct methods implemented in SHELXS-97. Intensity data for a total of 2682 unique reflections in a resolution range from 15.67-0.84 Å (2.82<θ<66.54) were used in the refinement of 263 crystallographic parameters by SHELXL-97. Final statistical parameters were: wR2=0.1749 (all data), R_{F}=0.0663 (data with I>2σ(I)) and goodness of fit S=1.035.

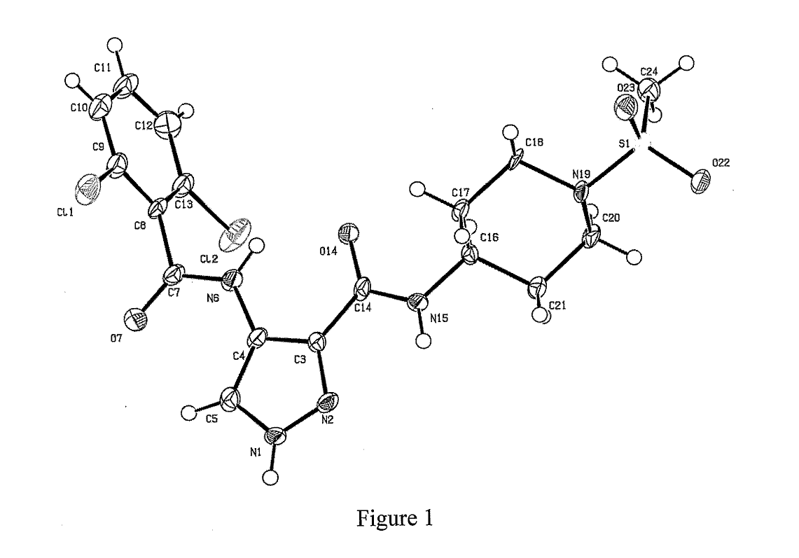

Only one molecule of free base was found in the asymmetric unit. The elemental composition of the asymmetric unit was C₁₇H₁₉Cl₂N₅O₄S and the calculated density of the crystals is 1.47 Mg/m³. Hydrogen atoms were generated on geometrical grounds while the location of heteroatom bound hydrogen atoms was confirmed by inspection of Fo-Fe difference maps. The positional and thermal parameters of hydrogen atoms were constricted to ride on corresponding non-hydrogen atoms. The thermal motion of non-hydrogen atoms was modelled by anisotropic thermal factors (see FIG. 1).

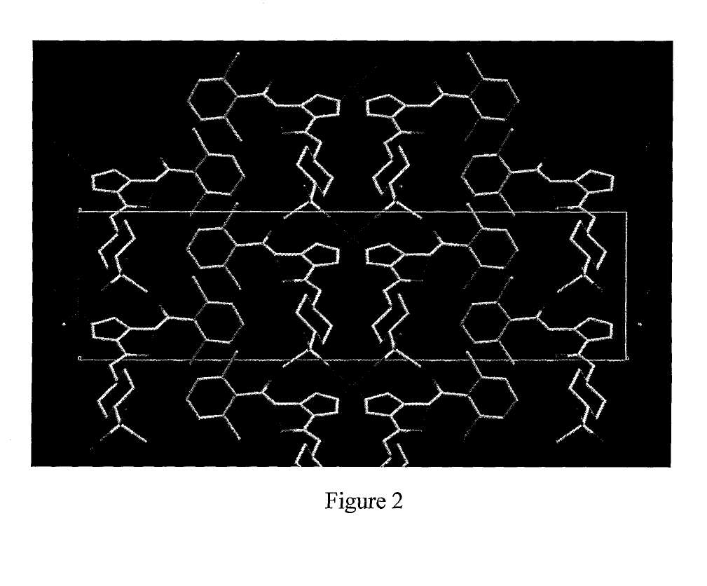

The crystal structure contains one intramolecular (N6-H . . . O14 2.812 Å) and one intermolecular hydrogen bond (see FIG. 2). The molecules are linked together into chains by intermolecular H-bond N1-H . . . O22 2.845 Å. Dichlorophenyl moieties from different chains stack together forming compact 3D packing.

A thermal ellipsoid representation of the structure generated by the X-ray diffraction study is provided in FIG. 1 and packing diagram is in FIG. 2.

The coordinates for the atoms making up the structure of the free base of 4-(2,6-dichlorobenzoylamino)-1H-pyrazole-3-carboxylic acid (1-methanesulphonyl-piperidin-4-yl)-amide are as set out in cif format in Table 1 below.

TABLE 1

space group: C2/c (# 15)
unit cell at 104K with a, b & c having 5% s.u.:
a = 9.150
b = 31.320
c = 7.930
alpha = gamma = 90.00
beta = 113.30
loop_{—}
_atom_site_label
_atom_site_type_symbol
_atom_site_fract_x
_atom_site_fract_y
_atom_site_fract_z
_atom_site_U_iso_or_equiv
_atom_site_adp_type
_atom_site_occupancy
_atom_site_symmetry_multiplicity
_atom_site_calc_flag
_atom_site_refinement_flags
_atom_site_disorder_assembly
_atom_site_disorder_group
Cl1 Cl 1.55055(16) 0.20997(4) 1.6202(2) 0.0376(4) Uani 1 1 d . . .
Cl2 Cl 0.97743(17) 0.20548(4) 1.6837(3) 0.0447(5) Uani 1 1 d . . .
S1 S 0.57041(12) 0.07771(3) 0.25572(15) 0.0212(3) Uani 1 1 d . . .
O7 O 1.3597(5) 0.14890(12) 1.8380(5) 0.0376(10) Uani 1 1 d . . .
O14 O 1.0227(4) 0.12633(10) 1.1610(5) 0.0266(8) Uani 1 1 d . . .
O22 O 0.4600(4) 0.04232(10) 0.1911(5) 0.0285(9) Uani 1 1 d . . .
O23 O 0.6695(4) 0.08741(13) 0.1578(5) 0.0282(9) Uani 1 1 d . . .
N1 N 1.2370(5) 0.02604(12) 1.5929(6) 0.0215(9) Uani 1 1 d . . .
H1 H 1.2665 0.0019 1.6538 0.026 Uiso 1 1 calc . . .
N2 N 1.1481(5) 0.02788(12) 1.4095(6) 0.0241(10) Uani 1 1 d . . .
N6 N 1.2053(5) 0.13987(12) 1.5365(6) 0.0226(9) Uani 1 1 d . . .
H6 H 1.1513 0.1533 1.4330 0.027 Uiso 1 1 calc . . .
N15 N 0.9606(5) 0.05870(11) 1.0508(6) 0.0192(9) Uani 1 1 d . . .
H15 H 0.9804 0.0313 1.0720 0.023 Uiso 1 1 calc . . .
N19 N 0.6881(4) 0.06785(12) 0.4705(5) 0.0185(9) Uani 1 1 d . . .
C3 C 1.1279(5) 0.06988(14) 1.3718(7) 0.0196(10) Uani 1 1 d . . .
C4 C 1.2051(5) 0.09437(14) 1.5332(7) 0.0210(10) Uani 1 1 d . . .
C5 C 1.2765(6) 0.06537(16) 1.6738(8) 0.0240(11) Uani 1 1 d . . .
H5 H 1.3393 0.0714 1.7992 0.029 Uiso 1 1 calc . . .
C7 C 1.2811(6) 0.16340(14) 1.6846(7) 0.0243(11) Uani 1 1 d . . .
C8 C 1.2638(7) 0.21135(14) 1.6550(8) 0.0239(11) Uani 1 1 d . . .
C9 C 1.3834(6) 0.23627(16) 1.6278(7) 0.0260(11) Uani 1 1 d . . .
C10 C 1.3723(7) 0.27967(18) 1.6094(8) 0.0331(13) Uani 1 1 d . . .
H10 H 1.4564 0.2955 1.5978 0.040 Uiso 1 1 calc . . .
C11 C 1.2352(7) 0.30098(16) 1.6076(8) 0.0333(14) Uani 1 1 d . . .
H11 H 1.2266 0.3311 1.5928 0.040 Uiso 1 1 calc . . .
C12 C 1.1136(7) 0.27794(18) 1.6273(8) 0.0354(14) Uani 1 1 d . . .
H12 H 1.0207 0.2921 1.6242 0.043 Uiso 1 1 calc . . .
C13 C 1.1291(6) 0.23383(16) 1.6518(8) 0.0321(14) Uani 1 1 d . . .
C14 C 1.0327(5) 0.08684(14) 1.1863(7) 0.0218(11) Uani 1 1 d . . .
C16 C 0.8492(5) 0.07270(14) 0.8678(7) 0.0184(10) Uani 1 1 d . . .
H16 H 0.7916 0.0985 0.8838 0.022 Uiso 1 1 calc . . .
C17 C 0.9342(5) 0.08479(14) 0.7426(7) 0.0211(11) Uani 1 1 d . . .
H17A H 0.9903 0.0595 0.7223 0.025 Uiso 1 1 calc . . .
H17B H 1.0142 0.1073 0.8019 0.025 Uiso 1 1 calc . . .
C18 C 0.8119(5) 0.10120(15) 0.5567(7) 0.0225(10) Uani 1 1 d . . .
H18A H 0.7612 0.1276 0.5760 0.027 Uiso 1 1 calc . . .
H18B H 0.8665 0.1080 0.4743 0.027 Uiso 1 1 calc . . .
C20 C 0.6048(5) 0.05454(15) 0.5920(7) 0.0242(11) Uani 1 1 d . . .
H20A H 0.5265 0.0319 0.5305 0.029 Uiso 1 1 calc . . .
H20B H 0.5466 0.0792 0.6132 0.029 Uiso 1 1 calc . . .
C21 C 0.7264(6) 0.03785(14) 0.7776(7) 0.0234(11) Uani 1 1 d . . .
H21A H 0.6712 0.0302 0.8584 0.028 Uiso 1 1 calc . . .
H21B H 0.7798 0.0120 0.7578 0.028 Uiso 1 1 calc . . .
C24 C 0.4560(6) 0.12321(16) 0.2544(8) 0.0279(12) Uani 1 1 d . . .
H24A H 0.5263 0.1479 0.2999 0.042 Uiso 1 1 calc . . .
H24B H 0.3984 0.1181 0.3338 0.042 Uiso 1 1 calc . . .
H24C H 0.3796 0.1288 0.1288 0.042 Uiso 1 1 calc . . .

### Example 3

### X-Ray Powder Diffraction (XRPD) Studies of Crystals of 4-(2,6-dichlorobenzoylamino)-1H-pyrazole-3-carboxylic acid (1-methanesulphonyl-piperidin-4-yl)-amide

Crystals of 4-(2,6-dichlorobenzoylamino)-1H-pyrazole-3-carboxylic acid (1-methanesulphonyl-piperidin-4-yl)-amide were prepared using the recrystallisation method described in Example 1 Step 8.

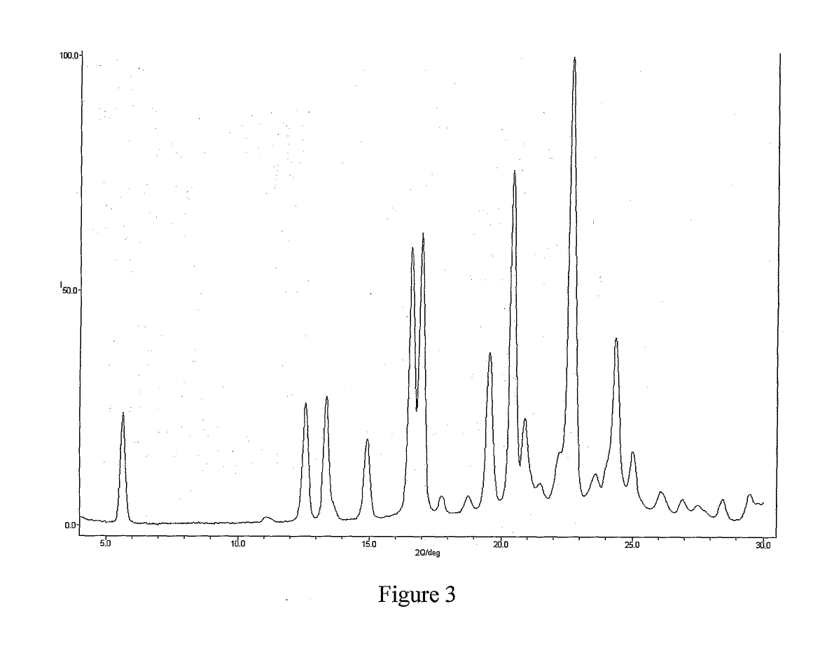

The crystal samples for X-ray powder diffraction (XRPD) data collection were gently ground by marble mortar and loaded into a crystallographic capillary (from Hampton Research, Quartz or Glass Type 10, 0.4 or 0.7 mm diameter). Diffraction patterns were collected at room temperature using CuKα radiation (λ=1.5418 Å) from a Rigaku rotating anode RU3HR, Osmic blue confocal optics, ¼χ goniometer and a Rigaku HTC image plate detector. 2D Images were collected while spinning φ axis with a detector to crystal distance of 250 mm. Data collection was controlled by CrystalClear software and 2D images were converted to 1D plot (2θ vs. Intensity) by Datasqueeze (intensity averaged over the azimuthal angle 0<χ<360° for 2θ range 3-30° in 0.01° or 0.02°steps). An in house program AstexXRPD was used for manipulation and visualisation of 1D XRPD patterns (FIG. 3).

TABLE 2
2θ, d-spacing and relative intensity of main peaks.

| 2θ/° | d/Å | I |
|---|---|---|
| 5.63 | 15.70 | 24 |
| 12.56 | 7.05 | 26 |
| 13.35 | 6.63 | 27 |
| 14.89 | 5.95 | 18 |
| 16.57 | 5.35 | 59 |
| 16.95 | 5.23 | 62 |
| 19.53 | 4.55 | 37 |
| 20.42 | 4.35 | 76 |
| 20.88 | 4.25 | 23 |
| 22.66 | 3.92 | 100 |
| 24.33 | 3.66 | 40 |
| 24.99 | 3.56 | 16 |

### Example 4

### Physicochemical Studies on 4-(2,6-dichlorobenzoylamino)-1H-pyrazole-3-carboxylic acid (1-methanesulphonyl-piperidin-4-yl)-amide

Crystals of 4-(2,6-dichlorobenzoylamino)-1H-pyrazole-3-carboxylic acid (1-methanesulphonyl-piperidin-4-yl)-amide prepared by the recrystallisation method of Example 1 Step 8 were subjected to differential scanning calorimetry studies and thermogravimetric analysis.

Differential Scanning Calorimetry Study

Approximately 1-3 mg of sample (accurately weighed) were placed into an aluminium DSC pan and crimped using an aluminium lid to ensure a tight seal. The sample was then placed into a Pyris Diamond DSC (Perkin-Elmer) equipped with a liquid nitrogen cooling unit and allowed to equilibrate at 25° C. until a stable heat flow response was seen. A dry helium purge gas at a flow rate of 20 ml/min was used to produce an inert atmosphere and prevent oxidation of the sample during heating. The sample was then scanned from 25-400° C. at a scan rate of 200° C./min and the resulting heat flow response (mW) measured against temperature. Prior to experimental analysis the instrument was temperature and heat-flow calibrated using an indium reference standard.

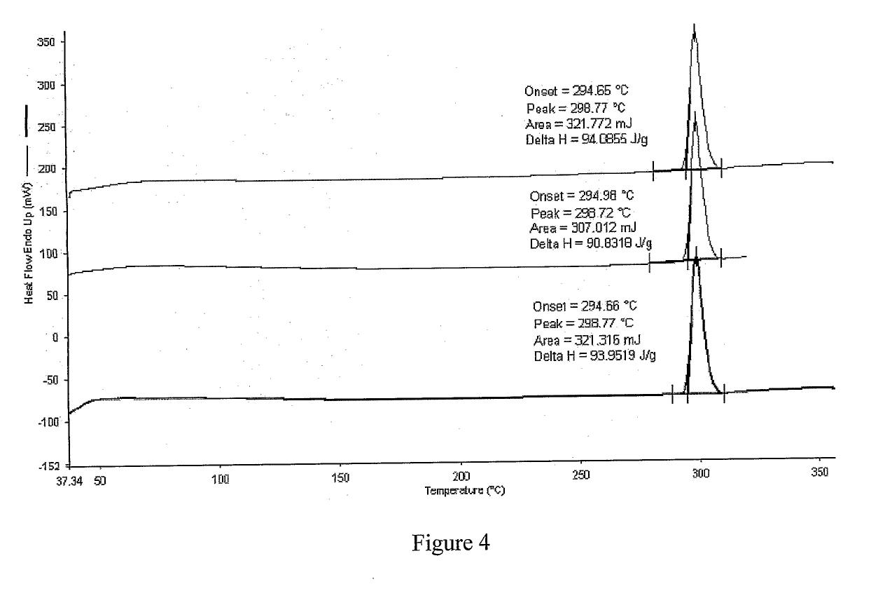

A DSC scan of the compound is shown in FIG. 4.

Thermogravimetric Analysis

Approximately 5 mg of sample (accurately weighed) was placed into a platinum TGA pan and loaded into a TGA 7 gravimetric analyser. The sample under study was then heated at a rate of 10° C./min (from ambient to 300° C.) and the resulting change in weight monitored. A dry nitrogen purge gas at a flow rate of 20 ml/min was used to produce an inert atmosphere and prevent oxidation of the sample during heating. Prior to analysis the instrument was weight calibrated using a 100 mg reference standard and temperature calibrated using an Alumel reference standard (using the Curie point transition temperature).

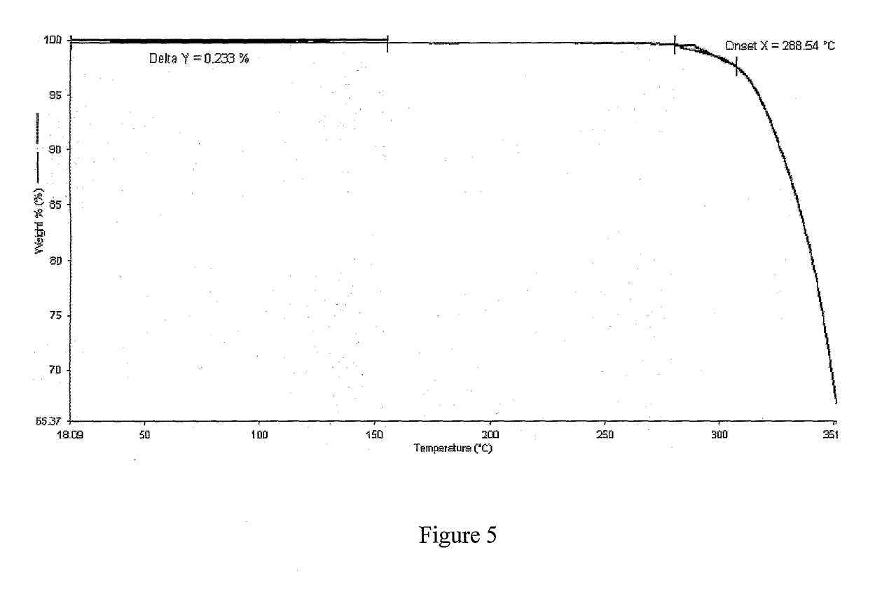

The weight loss profile of the compound is shown in FIG. 5.

Results and Conclusions

From the resulting DSC thermograms obtained, a single defined and co-operative endothermic transition was seen onset ca. 294.5-295° C., indicative of the thermally induced melting of the crystalline lattice. No significant transitions were apparent prior to the main melting endotherm, indicating little/no loss of chemisorbed (bound) volatiles from the sample (as a result of dehydration/desolvation) as well as no detectable presence of amorphous content. This lack of a hydrated or solvated state was confirmed using TGA (FIG. 5) which showed a mass loss of approximately 0.2% up to 150° C. This suggests the existence of this drug form in the solely anhydrous crystalline state with no detectable polymorphic impurities or polymorphic transformations occurring.

The TGA plot (FIG. 5), shows a significant event at about 288° C. which occurred with an onset prior to the main melt transition, suggesting a small degree of thermally induced partial degradation of the sample prior to and during the melt. This degradation process was accelerated at temperatures greater than 300° C.

### Example 5

### Vapour Sorption/Desorption Analysis of 4-(2,6-dichlorobenzoylamino)-1H-pyrazole-3-carboxylic acid (1-methanesulphonyl-piperidin-4-yl)-amide

Crystals of 4-(2,6-dichlorobenzoylamino)-1H-pyrazole-3-carboxylic acid (1-methanesulphonyl-piperidin-4-yl)-amide prepared by the recrystallisation method of Example 1 Step 8 were subjected to vapour sorption/desorption analysis in order to test for the propensity of this sample to form a hydrated state.

Approximately 20 mg of sample was placed into a wire-mesh vapour sorption balance pan and loaded into an ‘IgaSorp’ vapour sorption balance (Hiden Analytical Instruments) held at 25±0.1° C. The sample was then dried by maintaining a 0% humidity environment (using mass flow control apparatus) until no further weight change was recorded. Subsequently, the sample was then subjected to a ramping profile from 0-90% relative humidity (% RH) at 10% RH increments, maintaining the sample at each step until equilibration had been attained (99.5% step completion).

Upon reaching equilibration, the % RH within the apparatus was ramped to the next step and the equilibration procedure repeated. After completion of the sorption cycle, the sample was then dried using the same procedure. The weight change during the sorption/desorption cycles was then monitored, allowing for the hygroscopic nature of the sample to be determined.

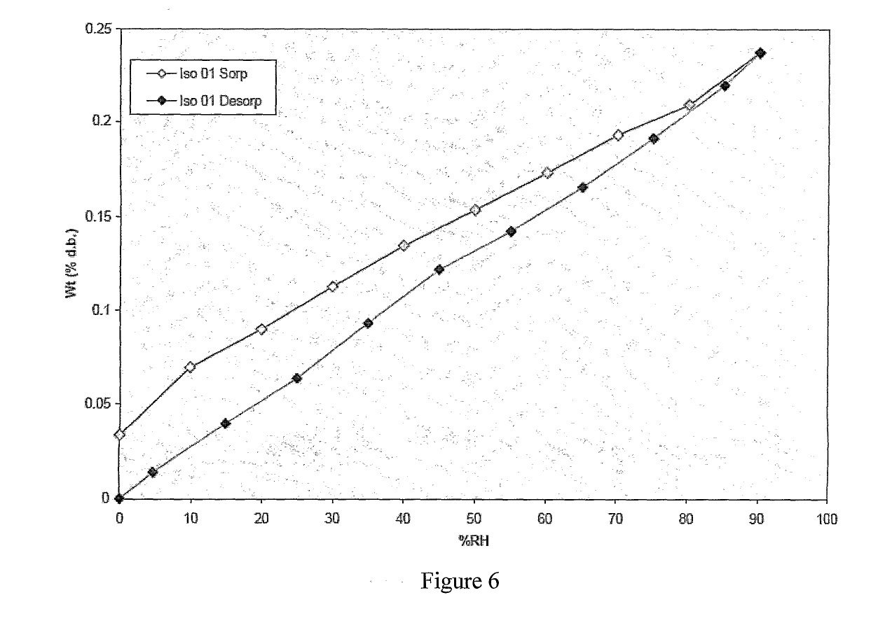

A vapour sorption/desorption profile of the compound is shown in FIG. 6.

During initial drying of the sample (at 0% RH), a weight loss of approximately 0.01% was seen, corresponding to the removal of loosely bound physi-sorbed or unbound surface adsorbed water present on the particles prior to analysis. Subsequently, increasing the relative humidity stepwise to 90% RH resulted in corresponding small incremental weight increases, totalling 0.24% upon equilibration at 90% RH. These small degrees of mass uptake seen upon storage at the varying humidities was the result of simple surface adsorption of a monolayer of water onto the particle surfaces with no true crystalline hydrate formation evident. This suggests that the compound is physically stable with regard to hygroscopicity and does not convert to the hydrated state upon storage in elevated humidity conditions.

Biological Activity

### Example 6

### Measurement of Activated CDK2/CyclinA Kinase Inhibitory Activity Assay (IC₅₀)

The compound of the invention were tested for kinase inhibitory activity using the following protocol.

Activated CDK2/CyclinA (Brown et al, Nat. Cell Biol., 1, pp 438-443, 1999; Lowe, E. D., et al Biochemistry, 41, pp 15625-15634, 2002) is diluted to 125 pM in 2.5× strength assay buffer (50 mM MOPS pH 7.2, 62.5 mM β-glycerophosphate, 12.5 mM EDTA, 37.5 mM MgCl₂, 112.5 mM ATP, 2.5 mM DTT, 2.5 mM sodium orthovanadate, 0.25 mg/ml bovine serum albumin), and 10 μl mixed with 10 μl of histone substrate mix (60 μl bovine histone H1 (Upstate Biotechnology, 5 mg/ml), 940 μl H₂O, 35 μCi γ³³P-ATP) and added to 96 well plates along with 5 μl of various dilutions of the test compound in DMSO (up to 2.5%). The reaction is allowed to proceed for 2 to 4 hours before being stopped with an excess of ortho-phosphoric acid (5 μl at 2%). γ³³P-ATP which remains unincorporated into the histone H1 is separated from phosphorylated histone H1 on a Millipore MAPH filter plate. The wells of the MAPH plate are wetted with 0.5% orthophosphoric acid, and then the results of the reaction are filtered with a Millipore vacuum filtration unit through the wells. Following filtration, the residue is washed twice with 200 μl of 0.5% orthophosphoric acid. Once the filters have dried, 20 μl of Microscint 20 scintillant is added, and then counted on a Packard Topcount for 30 seconds.

The % inhibition of the CDK2 activity is calculated and plotted in order to determine the concentration of test compound required to inhibit 50% of the CDK2 activity (IC₅₀).

### Example 7

### Measurement of Activated CDK1/CyclinB Kinase Inhibitory Activity Assay (IC₅₀)

CDK1/CyclinB assay is identical to the CDK2/CyclinA above except that CDK1/CyclinB (Upstate Discovery) is used and the enzyme is diluted to 6.25 nM.

The compounds of the invention has an IC₅₀ value of less than 1 μM in the CDK2 or CDK1 assay.

### Example 8

### GSK3-B Kinase Inhibitory Activity Assay

GSK3-β (Upstate Discovery) are diluted to 7.5 nM in 25 mM MOPS, pH 7.00, 25 mg/ml BSA, 0.0025% Brij-35, 1.25% glycerol, 0.5 mM EDTA, 25 mM MgCl₂, 0.025% β-mercaptoethanol, 37.5 mM ATP and and 10 μl mixed with 10 μl of substrate mix. The substrate mix for GSK3-β is 12.5 μM phospho-glycogen synthase peptide-2 (Upstate Discovery) in 1 ml of water with 35 μCi γ³³P-ATP. Enzyme and substrate are added to 96 well plates along with 5 μl of various dilutions of the test compound in DMSO (up to 2.5%). The reaction is allowed to proceed for 3 hours (GSK3-β) before being stopped with an excess of ortho-phosphoric acid (5 μl at 2%). The filtration procedure is as for Activated CDK2/CyclinA assay above.

### Example 9

### Anti-Proliferative Activity

The anti-proliferative activities of the compound of the invention can be determined by measuring the ability of the compound to inhibition of cell growth in a number of cell lines. Inhibition of cell growth is measured using the Alamar Blue assay (Nociari, M. M, Shalev, A., Benias, P., Russo, C. Journal of Immunological Methods 1998, 213, 157-167). The method is based on the ability of viable cells to reduce resazurin to its fluorescent product resorufin. For each proliferation assay cells are plated onto 96 well plates and allowed to recover for 16 hours prior to the addition of inhibitor compounds for a further 72 hours. At the end of the incubation period 10% (v/v) Alamar Blue is added and incubated for a further 6 hours prior to determination of fluorescent product at 535 nM ex/590 nM em. In the case of the non-proliferating cell assay cells are maintained at confluence for 96 hour prior to the addition of inhibitor compounds for a further 72 hours. The number of viable cells is determined by Alamar Blue assay as before. Cell lines can be obtained from the EC ACC (European Collection of cell Cultures).

In particular, the compound of the invention was tested against the HCT-116 cell line (EC ACC Reference: 91091005) derived from human colon carcinoma and was found to have an IC₅₀ value of less than 1 μM.

### Example 10

### Determination of Oral Bioavailability

The oral bioavailability of the compound of formula (I) may be determined as follows.

The test compound is administered as a solution both I.V. and orally to balb/c mice at the following dose level and dose formulations;
1 mg/kg IV formulated in 10%DMSO/90% (2-hydroxypropyl)-β-cyclodextrin (25% w/v); and
5 mg/kg PO formulated in 10% DMSO/20% water/70% PEG200.

At various time points after dosing, blood samples are taken in heparinised tubes and the plasma fraction is collected for analysis. The analysis is undertaken by LC-MS/MS after protein precipitation and the samples are quantified by comparison with a standard calibration line constructed for the test compound. The area under the curve (AUC) is calculated from the plasma level vs time profile by standard methods. The oral bioavailability as a percentage is calculated from the following equation:

$$\frac{AUCpo}{AUCiv} \times \frac{dose IV}{dosePO} \times 100$$

By following this protocol, the compound 4-(2,6-dichloro-benzoylamino)-1H-pyrazole-3-carboxylic acid (1-methanesulphonyl-piperidin-4-yl)-amide, was found to have 40-50% bioavailability when administered to mice by the oral route.

### Example 11

### Xenograph Studies

The compound 4-(2,6-dichloro-benzoylamino)-1H-pyrazole-3-carboxylic acid (1-methanesulphonyl-piperidin-4-yl)-amide has an anti-tumour action in nude mice engrafted with human tumour derived cell lines. Treatment with the compound causes inhibition of tumour growth in such xenografts implanted sub-cutaneously when dosed orally at doses which cause inhibition of the tumour biomarkers. These biomarkers include suppression of phosphorylation of substrates of the cyclin dependent kinases e.g. retinoblastoma protein. The compound is effective when given in a range of different schedules including chronic dosing for several weeks.

### Example 12

### Comparative Example

The biological activities of the compound of the invention, 4-(2,6-dichloro-benzoylamino)-1H-pyrazole-3-carboxylic acid (1-methanesulphonyl-piperidin-4-yl)-amide, which contains a 2,6-dichlorophenyl group, were compared with the biological activities of its 2,6-difluorophenyl analogue. The 2,6-difluorophenyl analogue, which is described in Example 131 in our earlier application PCT/GB2004/003179 (publication number WO 2005/012256), has the following structure

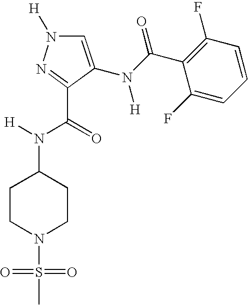

More particularly, the compounds were compared with regard to their activities against CDK2 kinase and GSK3β kinase and their ability to inhibit the proliferation of HCT-116 human colon cancer cells. The kinase inhibitory activities and the HCT-116 inhibitory activity were determined using the assay methods set out above and the results are shown in the table below.

| | Prior Art Compound | |
|---|---|---|
| | (Example 131 of | Compound of the |
| | PCT/GB2004/003179) | Invention |
| CDK2 IC₅₀ | 0.0022 uM | 43% @ 0.0003 μM |
| GSK3β IC₅₀ | 0.014 uM | 0.22 μM |
| HCT-116 cell | 0.74 uM | 0.11 μM |
| proliferation IC₅₀ | | |

The compound of the invention has advantages over the compound of its difluoro-analogue for the following reasons:
The compound of the invention has a 6-7-fold more potent anti-proliferative effect on human colon cancer HCT-116 cell line, when compared to its difluoro-analogue.
The compound of the invention has greater in vitro kinase (CDK2) inhibitory activity compared to its difluoro-analogue.
The compound of the invention has lower activity versus GSK3β (0.22 μM) than its difluoro-analogue (0.014 μM).
The compound of the invention has greater selectivity for CDK inhibition over GSK3β (>200-fold) compared to its difluoro-analogue (˜6-fold).

Pharmaceutical Formulations

### Example 13

(i) Tablet Formulation

A tablet composition containing a compound of the formula (I) is prepared by mixing 50 mg of the compound with 197 mg of lactose (BP) as diluent, and 3 mg magnesium stearate as a lubricant and compressing to form a tablet in known manner.

(ii) Capsule Formulation

A capsule formulation is prepared by mixing 100 mg of a compound of the formula (I) with 100 mg lactose and filling the resulting mixture into standard opaque hard gelatin capsules.

(iii) Injectable Formulation I

A parenteral composition for administration by injection can be prepared by dissolving a compound of the formula (I) (e.g. in a salt form) in water containing 10% propylene glycol to give a concentration of active compound of 1.5% by weight. The solution is then sterilised by filtration, filled into an ampoule and sealed.

(iv) Injectable Formulation II

A parenteral composition for injection is prepared by dissolving in water a compound of the formula (I) (e.g. in salt form) (2 mg/ml) and mannitol (50 mg/ml), sterile filtering the solution and filling into sealable 1 ml vials or ampoules.

(v) Injectable Formulation III

A formulation for i.v. delivery by injection or infusion can be prepared by dissolving the compound of formula (I) (e.g. in a salt form) in water at 20 mg/ml. The vial is then sealed and sterilised by autoclaving.

(vi) Injectable Formulation IV

A formulation for i.v. delivery by injection or infusion can be prepared by dissolving the compound of formula (I) (e.g. in a salt form) in water containing a buffer (e.g. 0.2 M acetate pH 4.6) at 20 mg/ml. The vial is then sealed and sterilised by autoclaving.

(vii) Subcutaneous Injection Formulation

A composition for sub-cutaneous administration is prepared by mixing a compound of the formula (I) with pharmaceutical grade corn oil to give a concentration of 5 mg/ml. The composition is sterilised and filled into a suitable container.

(viii) Lyophilised Formulation

Aliquots of formulated compound of formula (I) are put into 50 mL vials and lyophilized. During lyophilisation, the compositions are frozen using a one-step freezing protocol at (−45° C.). The temperature is raised to −10° C. for annealing, then lowered to freezing at −45° C., followed by primary drying at +25° C. for approximately 3400 minutes, followed by a secondary drying with increased steps if temperature to 50° C. The pressure during primary and secondary drying is set at 80 millitor.

(ix) Solid Solution Formulation

The compound of Example 1 and PVP are dissolved in dichloromethane/ethanol (1:1) at a concentration of 5 to 50% (for example 16 or 20%) and the solution is spray dried using conditions corresponding to those set out in the table below. The data given in the table include the concentration of the compound of Example 1, the inlet and outlet temperatures of the spray drier, the total yield of spray dried solid, the concentration of the compound of Example 1 in the spray dried solid (assay), and the particle size distribution (P.S.D.) of the particles making up the spray dried solid.

| | conc sol. | temp. | temp. | % | assay | PSD (range) |
|---|---|---|---|---|---|---|
| Batch | w/vol | inlet | outlet | yield | (mg/g) | (μm) |
| BR1A | 16% | 140° C. | 80° C. | 87.00 | 246.41 | 4.46-52.76 |
| BR1B | 16% | 180° C. | 80° C. | 97.00 | 246.65 | 14.83-91.70 |
| BR2A | 20% | 160° C. | 80° C. | 99.40 | 239.60 | 15.86-85.01 |
| BR3A | 20% | 180° C. | 100° C. | 79.50 | 246.64 | 15.09-91.84 |

The solid solution of the compound of Example 1 and PVP can either be filled directly into hard gelatin or HPMC (hydroxypropylmethyl cellulose) capsules, or be mixed with pharmaceutically acceptable excipients such as bulking agents, glidants or dispersants. The capsules could contain the compound of Example 1 in amounts of between 2 mg and 200 mg, for example 10, 20 and 80 mg. Alternatively the capsules could contain 40 mg of compound of the Example 1.

### Example 14

### Pharmaceutical Formulations Containing a Solid Dispersion of 4-(2,6-dichloro-benzoylamino)-1H-pyrazole-3-carboxylic acid (1-methanesulphonyl-piperidin-4-yl)-amide in Polyvinylpyrrolidone (PVP)

This example describes the preparation of granule compositions containing a spray dried solid dispersion of 4-(2,6-dichloro-benzoylamino)-1H-pyrazole-3-carboxylic acid (1-methanesulphonyl-piperidin-4-yl)-amide and the K30 grade of polyvinylpyrrolidone (Kollidon K30) available from BASF ChemTrade GmbH of Burgbernheim, Germany). The molecular weight of the PVP is in the range 44,000-54,000.

The solid dispersion was prepared by dissolving 4-(2,6-dichloro-benzoylamino)-1H-pyrazole-3-carboxylic acid (1-methanesulphonyl-piperidin-4-yl)-amide in a 1:1 (v/v) mixture of ethanol and dichloromethane to give a concentration of the compound of 50 mg/mL, and then adding PVP K30 in a ratio of compound to PVP of 1:3.

The solute was then spray dried in a Niro Mobile Minor 2000 spray dryer. The powder collected from the spray dryer was dried under vacuum.

The spray drying conditions were as follows:

| Nozzle internal diameter (ID): | 1 | mm |
|---|---|---|
| Tubing ID: | 3 | mm |
| Inlet temperature: | 180° | C. |
| Exhaust temperature: | 85° | C. |
| Atomisation pressure: | 1.0 | bar |
| Process gas flow: | 3.2 mbar (83 kg/h of nitrogen) | |
| Process gas: | nitrogen | |
| Solution dry weight (compound + PVP): | 1980 | g |
| Flow rate: | 123 | g/min |

| | |
|---|---|
| Yield: | 84.85% |

The particle size distribution of the spray dried solid dispersion, following drying, was measured using a laser diffraction apparatus and gave D10, D50 and D90 figures as follows:

| | |
|---|---|
| D10/μm | 17.53 |
| D50/μm | 49.08 |
| D90/μm | 93.26 |

In the following example, the solid dispersion of 4-(2,6-dichloro-benzoylamino)-1H-pyrazole-3-carboxylic acid (1-methanesulphonyl-piperidin-4-yl)-amide in PVP is referred to as “Compound of formula (I)/PVP”.

The following materials were blended for 30 seconds in a high shear mixer:—

| | |
|---|---|
| Dicalcium phosphate (Emcompress ™) | 32.8 g |
| Silicified microcrystalline cellulose (ProSolv HD90 ™) | 10.9 g |
| Compound of formula (I)/PVP | 35.2 g |
| Croscarmellose sodium (Ac-Di-Sol ™) | 11.1 g |

The powder blend was then compressed using a Freund roller compactor. The following settings were required to produce a ribbon:—

| | | |
|---|---|---|
| Feed speed: | 60 | rpm |
| Roller speed: | 2 | rpm |
| Roller pressure: | 180 | kgf/cm² |

The ribbon of compressed powder was ground through a 710 μm sieve and the resulting granules were collected in a suitable container. An aliquot of the granule mass (9.0 g) was mixed with a further aliquot of Ac-Di-Sol (1.0 g). The quantity of the granule mass that could be filled into size 0 capsules was determined (both flush-filled and tightly packed). Results are summarised below.

Capsule fill weight

| Flush-filled | Tightly packed |
|---|---|
| 282 mg (24.8 mg compound) | 431 mg (37.9 mg) |

Disintegration Tests

For rapid release oral formulations, it is desirable that disintegration of the dosage form and release of the active ingredient should occur within 15 minutes. The capsule formulation described was therefore subjected to disintegration testing using a standard tablet/capsule disintegration apparatus (European Pharmacopoeia, 4^{th} Edition). Distilled water was used as the disintegration medium. The volume of the disintegration medium was 800 mL and the temperature was maintained at 37° C. (±1° C.). The assessment of dispersion/dissolution behaviour of the formulation was made by observation alone. The disintegration times are set out in the table below.

| Quantity of Compound of | |
|---|---|
| formula (I) per capsule (mg) | Disintegration time (min) |
| 24.8 (flush-filled) | 4 |
| 37.9 (tightly packed) | 5 |

Dissolution Testing

The rate of dissolution of the capsule formulation was compared with the rate of dissolution of (1) the non-encapsulated solid dispersion of PVP and the compound of formula (I) containing no further excipients and (2) the solid dispersion (1) packed tightly into a size 0 capsule and (3) the formulated sample.

The dissolution testing was conducted using the paddle apparatus as described in the European Pharmacopoeia, 4^{th} Edition.

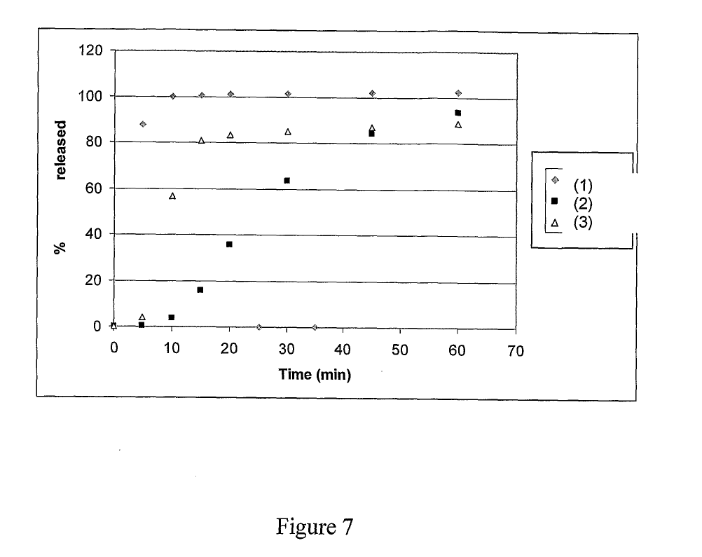

The results of the dissolution studies are shown in FIG. 7.

The results show that dissolution of the non-encapsulated solid dispersion was quicker than the dissolution of the capsule sample. In the tightly packed encapsulated sample, the PVP is probably binding the particles together, thus retarding the release of the compound of formula (I). Interestingly, the formulated sample exhibited a much more rapid compound release profile compared with the non-formulated, encapsulated sample, which indicates that the high proportion of disintegrant in the formulation is effective in countering the binding capacity of the PVP.

### Example 15

### Process for the Preparation of 4-(2,6-dichloro-benzoylamino)-1H-pyrazole-3-carboxylic acid (1-methanesulphonyl-piperidin-4-yl)-amide

Step 1—To a solution of 4-piperidone monohydrate hydrochloride (0.50 g, 3.25 mmol) in DMF (10 mL) was added triethylamine (2.44 mL, 17.6 mmol) and the mixture heated at 45° C. for 1 h. To the mixture was added methanesulphonyl chloride (0.75 mL, 9.75 mmol) and the mixture heated at 45° C. for 18 h. The resultant mixture was filtered and the filtrate reduced in vacuo. The residue was taken up in EtOAc and washed with water, the organic portion dried over MgSO₄ and reduced in vacuo to give 1-methanesulphonyl-piperidin-4-one as a pale yellow solid (369 mg).

Step 2—To a solution of 1-methanesulfonyl-piperidin-4-one (130 mg, 0.73 mmol) in DCM (3 mL) was added glacial acetic acid (32 μL, 0.55 mmol), benzylamine (108 μl, 0.99 mmol) and NaBH(OAc)₃ (232 mg, 1.09 mmol). The reaction mixture was stirred at ambient for 18 h. 2M Aqueous NaOH (3 mL) was added to the mixture and the layers separated. The organic portion was dried over MgSO₄ and reduced in vacuo to give 4-benzyloxy-1-methanesulfonyl-piperidine (160 mg) as a yellow solid.

Step 3—The transformation of 4-benzyloxy-1-methanesulfonyl-piperidine to produce 1-methanesulfonyl-piperidin-4-ylamine may be accomplished by dissolving 4-benzyloxy-1-methanesulfonyl-piperidine in an appropriate solvent and subjecting to an atmosphere of hydrogen in the presence of Pd/C.

Step 4:

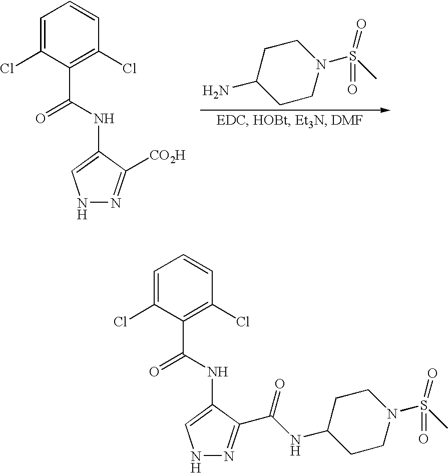

A mixture of 4-(2,6-dichloro-benzoylamino)-1H-pyrazole-3-carboxylic acid (3.6 g), 1-methanesulfonyl-piperidin-4-ylamine trifluoroacetate salt (3.53 g; 1.15 equiv.), EDC (2.87 g; 1.25 equiv.), HOBt (2.02 g; 1.25 equiv.) and triethylamine (3.5 ml; 2.1 equiv.) in DMF (50 ml) was stirred at r.t. for 20 h, then reduced in vacuo. The residue was triturated with sat NaHCO₃ (250 ml), solid collected by filtration, washed with water and sucked dry. Purification by hot slurry with EtOAc and chromatography eluting with EtOAc/P.E. (1:1 then 1:0) gave 2.8 g (51%) of (4-(2,6-dichloro-benzoylamino)-1H-pyrazole-3-carboxylic acid (1-methanesulfonyl-piperidin-4-yl)-amide as a white solid.

### Example 17

The formulated product of Example 14 was prepared through dry granulation of a solid dispersion of Compound 1 in PVP (ratio Compound 1:PVP of 1:3) with pharmaceutically acceptable excipients. This formulated product material was filled into size 0 capsule shells to give a dose equivalent to 10 mg and 40 mg of Compound 1. These capsules were placed on stability under two different storage conditions, 25° C./60% relative humidity (RH) and 40° C./75% relative humidity. The data below indicate that the formulated capsules have good physical and chemical stability, and consistent disintegration characteristics under these storage conditions.

Summary of Stability Data for 10 mg Formulated Capsules Stored in Blister Strips

| T (° C.)/ | | | | | Total | Water | |
|---|---|---|---|---|---|---|---|
| RH | Weeks | Appearance | Identity | Assay | Impurities | Content | Disintegration |
| 0 | 0 | White | +ve | 97.3% | 0.61% | 4.3% | 3 min 40 sec |
| | | capsules | | | | | |
| | | containing a | | | | | |
| | | white powder | | | | | |
| 25/60 | 6 | White | +ve | 96.3% | 0.70% | 4.4% | 2 min 55 sec |
| | | capsules | | | | | |
| | | containing a | | | | | |
| | | white powder | | | | | |
| 25/60 | 12 | White | +ve | 96.3% | 0.76% | 4.4% | 1 min 57 sec |
| | | capsules | | | | | |
| | | containing a | | | | | |
| | | white powder | | | | | |
| 25/60 | 26 | White | +ve | 98.1% | 1.01% | 4.8% | 2 min 51 sec |
| | | capsules | | | | | |
| | | containing a | | | | | |
| | | white powder | | | | | |
| 25/60 | 39 | White | +ve | 98.7% | 0.67% | 4.7% | 2 min 48 sec |
| | | capsules | | | | | |
| | | containing a | | | | | |
| | | white powder | | | | | |
| 40/75 | 6 | White | +ve | 96.2% | 0.69% | 5.5% | 3 min 24 sec |
| | | capsules | | | | | |
| | | containing a | | | | | |
| | | white powder | | | | | |
| 40/75 | 12 | White | +ve | 98.8% | 0.78% | 6.1% | 1 min 57 sec |
| | | capsules | | | | | |
| | | containing a | | | | | |
| | | white powder | | | | | |
| 40/75 | 26 | White | +ve | 98.6% | 0.97% | 7.3% | 3 min 02 sec |
| | | capsules | | | | | |
| | | containing a | | | | | |
| | | white powder | | | | | |

Summary of Stability Data for 40 mg Formulated Capsules Stored in Blister Strips

| T (° C.)/ | | | | | Total | Water | |
|---|---|---|---|---|---|---|---|
| RH | Weeks | Appearance | Identity | Assay | Impurities | Content | Disintegration |
| 0 | 0 | White | +ve | 97.9% | 0.63% | 4.8% | 3 min 24 sec |
| | | capsules | | | | | |
| | | containing a | | | | | |
| | | white powder | | | | | |
| 25/60 | 6 | White | +ve | 98.7% | 0.67% | 2.3% | 1 min 55 sec |
| | | capsules | | | | | |
| | | containing a | | | | | |
| | | white powder | | | | | |
| 25/60 | 12 | White | +ve | 98.6% | 0.75% | 2.6% | 1 min 53 sec |
| | | capsules | | | | | |
| | | containing a | | | | | |
| | | white powder | | | | | |
| 25/60 | 26 | White | +ve | 100.4% | 1.04% | 3.3% | 2 min 54 sec |
| | | capsules | | | | | |
| | | containing a | | | | | |
| | | white powder | | | | | |
| 25/60 | 39 | White | +ve | 99.5% | 0.66% | 2.0% | 3 min 15 sec |
| | | capsules | | | | | |
| | | containing a | | | | | |
| | | white powder | | | | | |
| 40/75 | 6 | White | +ve | 98.5% | 0.68% | 3.0% | 2 min 12 sec |
| | | capsules | | | | | |
| | | containing a | | | | | |
| | | white powder | | | | | |
| 40/75 | 12 | White | +ve | 98.9% | 0.80% | 10.4% | 1 min 22 sec |
| | | capsules | | | | | |
| | | containing a | | | | | |
| | | white powder | | | | | |
| 40/75 | 26 | White | +ve | 98.5% | 1.05% | 6.4% | 3 min 09 sec |
| | | capsules | | | | | |
| | | containing a | | | | | |
| | | white powder | | | | | |

Equivalents

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1-56. (canceled)

57. 4-(2,6-Dichloro-benzoylamino)-1H-pyrazole-3-carboxylic acid (1-methanesulphonyl-piperidin-4-yl)-amide in a substantially crystalline form.

58. 4-(2,6-Dichloro-benzoylamino)-1H-pyrazole-3-carboxylic acid (1-methanesulphonyl-piperidin-4-yl)-amide according to claim 57 which is at least 55% crystalline, or at least 60% crystalline, or at least 65% crystalline, or at least 70% crystalline, or at least 75% crystalline, or at least 80% crystalline, or at least 85% crystalline, or at least 90% crystalline, or at least 95% crystalline, or at least 98% crystalline, or at least 99% crystalline, or at least 99.5% crystalline, or at least 99.9% crystalline.

59. A substantially crystalline form of the compound 4-(2,6-dichloro-benzoylamino)-1H-pyrazole-3-carboxylic acid (1-methanesulphonyl-piperidin-4-yl)-amide according to claim 57 comprising a single crystalline form of a dehydrate of the compound and no more than 5% by weight of any other crystalline forms of the compound.

60. A crystalline form of 4-(2,6-dichloro-benzoylamino)-1H-pyrazole-3-carboxylic acid (1-methanesulphonyl-piperidin-4-yl)-amide according to claim 57 which is characterised by any one or more (in any combination) or all of the following parameters, namely that the crystalline form:
(a) as a crystal structure as set out in FIGS. 1 and 2; and/or
(b) has a crystal structure as defined by the coordinates in Table 1 herein; and/or
(c) has crystal lattice parameters at a=9.15, b=31.32, c=7.93 Å, β=113.3°, α=γ=90°; and/or
(d) has a crystal structure that belongs belong to a monoclinic space group; and/or
(e) has an X-ray powder diffraction pattern characterised by the presence of major peaks at the diffraction angles (2θ) and interplanar spacings (d) set forth in Table A, and optionally Table B; and/or
(f) exhibits peaks at the same diffraction angles as those of the X-ray powder diffraction pattern shown in FIG. 3; and/or
(g) has an X-ray powder diffraction pattern substantially as shown in FIG. 3; and/or
(h) is anhydrous and exhibits an endothermic peak at an endothermic peak at 293-296° C. when subjected to DSC; and/or
(i) exhibits an infra-red spectrum, when analysed using the UATR method, that contains characteristic peaks at containing characteristic peaks at 3362, 3019, 2843, 1677, 1577, 1547, 1533, 1326, 1150, 926, 781, 667 cm⁻¹.

61. A process for preparing 4-(2,6-dichloro-benzoylamino)-1H-pyrazole-3-carboxylic acid (1-methanesulphonyl-piperidin-4-yl)-amide, which process comprises reacting a compound of formula (II): 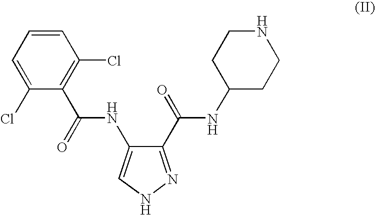
with methanesulphonyl chloride in a polar solvent in the presence of a base selected from alkali metal carbonates and bicarbonates; and thereafter isolating and optionally recrystallising the 4-(2,6-dichloro-benzoylamino)-1H-pyrazole-3-carboxylic acid (1-methanesulphonyl-piperidin-4-yl)-amide thus formed.

62. A process for the preparation of 4-(2,6-dichloro-benzoylamino)-1H-pyrazole-3-carboxylic acid (1-methanesulphonyl-piperidin-4-yl)-amide, which process comprises:
(a) reacting a compound of the formula (III) with methanesulphonic acid in a polar solvent to remove the boc group and give a methanesulphonate salt of a compound of the formula (II): 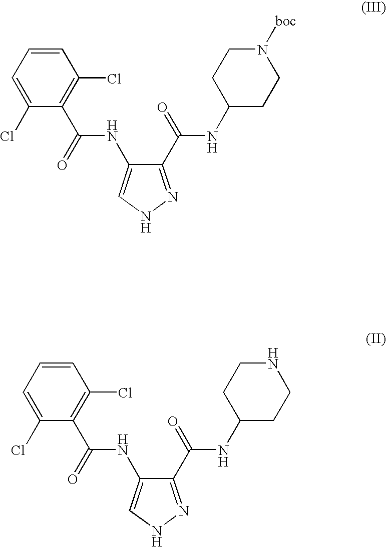
(b) isolating the methanesulphonate salt of the compound of formula (II);
(c) treating the methanesulphonate salt of the compound of formula (II) with methanesulphonic acid in an polar solvent to convert remaining traces of compound (III) to compound (II); and
(d) reacting the product of step (c) with methanesulphonyl chloride in a polar solvent in the presence of a base selected from alkali metal carbonates and bicarbonates; and thereafter isolating and optionally recrystallising the 4-(2,6-dichloro-benzoylamino)-1H-pyrazole-3-carboxylic acid (1-methanesulphonyl-piperidin-4-yl)-amide thus formed.

63. A process for the preparation of 4-(2,6-dichloro-benzoylamino)-1H-pyrazole-3-carboxylic acid (1-methanesulphonyl-piperidin-4-yl)-amide, which process comprises:
(ia) reacting an acid chloride compound of the formula (IV) with a compound of the formula (V): 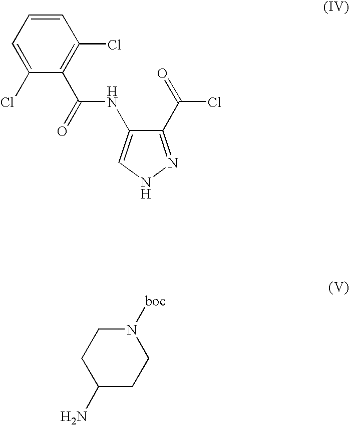
in a polar solvent in the presence of a base to give a compound of the formula (III): 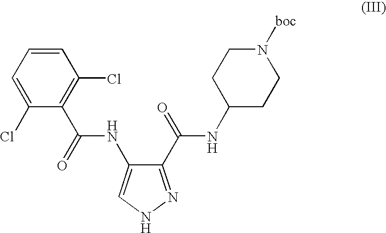
(a) reacting a compound of the formula (III) with methanesulphonic acid in a polar solvent to remove the boc group and give a methanesulphonate salt of a compound of the formula (II) 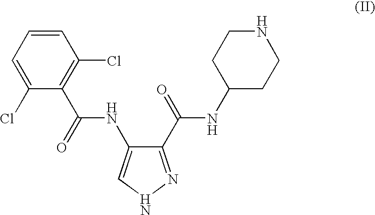
(b) isolating the methanesulphonate salt of the compound of formula (II);
(c) treating the methanesulphonate salt of the compound of formula (II) with methanesulphonic acid in an polar solvent to convert remaining traces of compound (III) to compound (II); and
(d) reacting the product of step (c) with methanesulphonyl chloride in a polar solvent in the presence of a base selected from alkali metal carbonates and bicarbonates; and thereafter isolating and optionally recrystallising the 4-(2,6-dichloro-benzoylamino)-1H-pyrazole-3-carboxylic acid (1-methanesulphonyl-piperidin-4-yl)-amide thus formed.

64. A process for the preparation of 4-(2,6-dichloro-benzoylamino)-1H-pyrazole-3-carboxylic acid (1-methanesulphonyl-piperidin-4-yl)-amide, which process comprises the reaction of a compound of the formula (VI) with 2,6-dichlorobenzoic acid or an activated derivative thereof

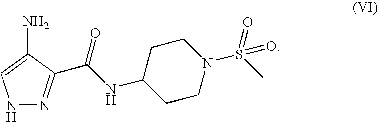

65. A solid pharmaceutical composition comprising a compressed mixture of:
(a) a solid dispersion of 4-(2,6-Dichloro-benzoylamino)-1H-pyrazole-3-carboxylic acid (1-methanesulphonyl-piperidin-4-yl)-amide in polyvinylpyrrolidone;
(b) a solid diluent;
(c) a disintegrant; and optionally
(d) one or more further pharmaceutically acceptable excipients.

66. A solid pharmaceutical composition according to claim 65 wherein
(i) the solid dispersion contains 4-(2,6-Dichloro-benzoylamino)-1H-pyrazole-3-carboxylic acid (1-methanesulphonyl-piperidin-4-yl)-amide and PVP in a weight ratio of about 1:1 to about 1:6;
(ii) the solid diluent is a pharmacologically inert solid substance chosen from sugars or sugar alcohols, and non-sugar derived diluents selected from sodium carbonate, calcium phosphate, calcium carbonate, cellulose or derivatives thereof and starches; and
(iii) the disintegrant is selected from cross linked carboxymethylcellulose (Croscarmellose), cross-linked polyvinylpyrrolidone (cross-linked PVP or Crospovidone), and sodium starch glycolate; and
(iv) the composition optionally contains one or more further pharmaceutically acceptable excipients (d) selected from microcrystalline cellulose, silicified microcrystalline cellulose and alkali metal bicarbonates.

67. A solid pharmaceutical composition according to claim 65 wherein:
component (a) is a spray dried solid dispersion of 4-(2,6-dichloro-benzoylamino)-1H-pyrazole-3-carboxylic acid (1-methanesulphonyl-piperidin-4-yl)-amide in PVP in a ratio of 1:3;
component (b) is calcium phosphate;
component (c) is Croscarmellose; and
component (d) is silicified microcrystalline cellulose.

68. A solid pharmaceutical composition according to claim 65 comprising a mixture of:
(a) 10-70% w/w of solid dispersion of 4-(2,6-dichloro-benzoylamino)-1H-pyrazole-3-carboxylic acid (1-methanesulphonyl-piperidin-4-yl)-amide in polyvinylpyrrolidone;
(b) 10-70% w/w of a solid diluent: and
(c) 1-20% w/w of a disintegrant; and optionally
(d) 1-30% w/w of one or more further pharmaceutically acceptable excipients.

69. A method of inhibiting tumour growth in a mammal, which method comprises administering to the mammal an effective tumour growth-inhibiting amount of 4-(2,6-Dichloro-benzoylamino)-1H-pyrazole-3-carboxylic acid (1-methanesulphonyl-piperidin-4-yl)-amide in a substantially crystalline form as defined in claim 57.

70. A method for treating, or alleviating or reducing the incidence of, a disease or condition comprising or arising from abnormal cell growth in a mammal, which method comprises administering to the mammal 4-(2,6-dichloro-benzoylamino)-1H-pyrazole-3-carboxylic acid (1-methanesulphonyl-piperidin-4-yl)-amide in a substantially crystalline form as defined in claim 57, in an amount effective in inhibiting abnormal cell growth.

71. A method according to claim 70 wherein the disease or condition is a cancer selected from a carcinoma of the bladder, breast, colon, kidney, epidermis, liver, lung, oesophagus, gall bladder, ovary, pancreas, stomach, cervix, thyroid, prostate, or skin; a hematopoietic tumour of lymphoid lineage; a hematopoietic tumour of myeloid lineage; a tumour of mesenchymal origin; a tumour of the central or peripheral nervous system; melanoma; seminoma; teratocarcinoma; osteosarcoma; xeroderma pigmentosum; keratoctanthoma; thyroid follicular cancer; and Kaposi's sarcoma.

72. A method for the prophylaxis or treatment of a disease state or condition mediated by a cyclin dependent kinase or glycogen synthase kinase-3, which method comprises administering to a subject in need thereof 4-(2,6-Dichloro-benzoylamino)-1H-pyrazole-3-carboxylic acid (1-methanesulphonyl-piperidin-4-yl)-amide in a substantially crystalline form as defined in claim 57.

73. A method of inhibiting a cyclin dependent kinase or glycogen synthase kinase-3, which method comprises contacting the kinase with 4-(2,6-Dichloro-benzoylamino)-1H-pyrazole-3-carboxylic acid (1-methanesulphonyl-piperidin-4-yl)-amide in a substantially crystalline form as defined in claim 57.

74. A pharmaceutical composition comprising 4-(2,6-Dichloro-benzoylamino)-1H-pyrazole-3-carboxylic acid (1-methanesulphonyl-piperidin-4-yl)-amide in a substantially crystalline form as defined in claim 57 and a pharmaceutically acceptable carrier.

75. A method for the diagnosis and treatment of a disease state or condition mediated by a cyclin dependent kinase, which method comprises (i) screening a patient to determine whether a disease or condition from which the patient is or may be suffering is one which would be susceptible to treatment with a compound having activity against cyclin dependent kinases; and (ii) where it is indicated that the disease or condition from which the patient is thus susceptible, thereafter administering to the patient 4-(2,6-Dichloro-benzoylamino)-1H-pyrazole-3-carboxylic acid (1-methanesulphonyl-piperidin-4-yl)-amide in a substantially crystalline form as defined in claim 57.

76. A process for preparing 4-(2,6-Dichloro-benzoylamino)-1H-pyrazole-3-carboxylic acid (1-methanesulphonyl-piperidin-4-yl)-amide, which process comprises reacting a carboxylic acid of formula (XII): 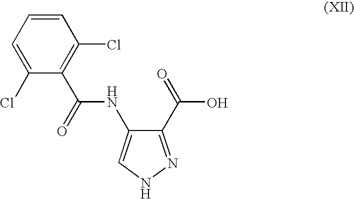
or an activated derivative thereof, with a compound of the formula (XIII): [chemical structure]
